(19) United States
(12) Patent Application Publication
(10) Pub. No.: US 2016/0000936 A1
(43) Pub. Date: Jan. 7, 2016

(54) BIOMARKERS FOR INFLAMMATORY DISEASE AND METHODS OF USING SAME

(71) Applicant: AbbVie Inc., North Chicago, IL (US)

(72) Inventors: Carolyn Cuff, Grafton, MA (US); Melanie C. Ruzek, Acton, MA (US); Jeffrey W. Voss, Holden, MA (US)

(21) Appl. No.: 14/736,256

(22) Filed: Jun. 10, 2015

Related U.S. Application Data
(60) Provisional application No. 62/010,438, filed on Jun. 10, 2014.
(60) Provisional application No. 62/013,342, filed on Jun. 17, 2014.
(60) Provisional application No. 62/016,083, filed on Jun. 23, 2014.
(60) Provisional application No. 62/080,088, filed on Nov. 14, 2014.

(51) Int. Cl. A61K 49/00; G01N 33/68; C07K 16/24

(52) U.S. Cl. CPC A61K 49/0008; C07K 16/241; C07K 16/244; G01N 33/6863; G01N 33/6866; G01N 33/6869; A61K 2039/507

(57) ABSTRACT

The invention provides methods for predicting the efficacy of anti-TNF and anti-IL-17 combination therapies in the treatment of a subject suffering from inflammatory disease by determining the level LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof markers in a sample derived from the subject.

### RELATED APPLICATIONS

This application is related to U.S. provisional application Ser. No. 62/080,088 filed Nov. 14, 2014, U.S. provisional application Ser. No. 62/016,083, filed Jun. 23, 2014, U.S. provisional application Ser. No. 62/013,342, filed Jun. 17, 2014, and U.S. provisional application Ser. No. 62/010,438, filed Jun. 10, 2014, each of which is incorporated herein by reference in its entirety.

### BACKGROUND OF THE INVENTION

Anti-cytokine therapies have become the standard of care for treating the symptoms and arresting the disease progression of inflammatory diseases. But despite the numerous treatment options, many patients still fail to experience a substantial decrease in disease activity. In principle, increasing the level of immunosuppression by combining agents is a plausible strategy for achieving improved efficacy. But attempts to combine anti-cytokine therapies to this end have been plagued by unacceptable safety and tolerability issues. Nevertheless, finding a combination therapy for the treatment of inflammatory disease that provides both an improved response and acceptable safety remains a challenge.

Rheumatoid arthritis (RA) is a chronic systemic autoimmune disease with unknown etiology. Its primary organ manifestations include joint inflammation resulting in pain, swelling and progressive bone and cartilage destruction, with numerous co-morbidities that include anemia and increased risk of cardiovascular events. As of 2012, over 5 million people were afflicted with RA, with approximately 26% having mild, 49% moderate, and 25% severe disease, with women being affected three (3) times more than men. In many cases, current treatment regimens are not completely efficacious.

Anti-tumor necrosis factor (TNF) therapies are the most prescribed anti-cytokine therapies for RA. TNF is a pro-inflammatory cytokine that triggers the acute phase response and increases expression of many mediators of pain, inflammation and joint destruction including other inflammatory cytokines and matrix metalloproteases and activate several pathways, including the NF-κB, MAPK, and apopotosis pathways. In many RA patients that fail to achieve remission, and in rodent disease models, anti-TNF therapy is only partially effective in suppressing the effects of this pro-inflammatory cytokine. Based on a number of in vitro studies, TNF appears to cooperate with IL-17 in regulating pro-inflammatory gene expression, making the dual anti-TNF/anti-IL-17 treatment an attractive combination therapy.

It remains to be seen whether the dual inhibition of TNF and IL-17 will be safe and effective in all patients. Biomarkers are typically used as measurable indicators of disease severity or progression, and to evaluate the most effective therapeutic regimen for the treatment of diseases. Biomarkers in the context of drug development include changes in the expression patterns of certain gene products, such as an increase or decrease in the level of a certain protein in the serum. In particular, biomarkers can be used to predict whether a drug will be effective in a particular patient or patient population and to tailor a patient's treatment options. Whereas a number of biomarkers are available to the clinician as a general indicator of inflammation, the efficacy of, or response to, certain anti-inflammatory treatments can be indicated by a particular biomarker(s).

Accordingly, there is a need in the art for measurable indicators of drug efficacy as well as methods for assessing or predicting responsiveness to combined inflammatory disease therapies comprising anti-TNF and anti-IL-17.

### SUMMARY OF THE INVENTION

An aspect of the invention provides a method of determining the suitability of a subject suffering from an inflammatory disorder for treatment with a combination therapy comprising an anti-TNF treatment and an anti-IL-17 treatment, the method comprising contacting a sample from a first subject with one or more binding moieties that specifically bind a protein or a nucleic acid that encodes the protein, wherein the protein is selected from the group consisting of: LIF, C-X-C motif chemokine 1 (CXCL1), CXCL2, CXCL4, CXCL5, CXCL8, CXCL9, CXCL10, chemokine (C-C motif) ligand 2 (CCL2), CCL23, interleukin-1 beta (IL-1β), IL-1 receptor antagonist (IL-1Ra), TNF, IL-6, IL-10, IL-17A, IL-17F, IL-21, IL-22, interferon gamma (IFNγ), C-X-C chemokine receptor type 1 (CXCR1), CXCR4, CXCR5, granulocyte-macrophage colony-stimulating factor (GM-CSF), GM-CSF receptor (GM-CSFR), granulocyte-colony stimulating factor (G-CSF), G-CSF receptor (G-CSFR) protein or nucleic acid, or a homolog, portion or derivative thereof; detecting the interaction of the one or more binding moieties with the protein or the nucleic acid, thereby detecting the relative abundance of the protein or the nucleic acid in the first subject sample; comparing the relative abundance of the protein or the nucleic acid to the relative abundance of protein or nucleic acid in a second subject sample, wherein the second subject does not suffer from the inflammatory disorder and the protein or nucleic acid in the second subject sample corresponds to the protein or the nucleic acid from the first subject sample; and selecting the first subject for the combination therapy comprising an anti-TNF treatment and an anti-IL-17 treatment if the abundance of the protein or nucleic acid in the first subject sample is altered relative to the abundance of the protein or nucleic acid in the second subject sample. In an embodiment, the relative abundance of the protein or nucleic acid in the first subject sample is higher than the relative abundance of the protein or nucleic acid in the second subject sample. Alternatively, the relative abundance of the protein or nucleic acid in the first subject sample is lower than the relative abundance of the protein or nucleic acid in the second subject sample.

In certain embodiments, LIF, IL-1 RA, IL-10, IL-21 and CXCR5 are increased in abundance in subjects in response to administration of an anti-TNF treatment and an anti-IL-17 treatment. Thus, if these biomarkers have low abundance in a subject with an inflammatory disorder relative to a healthy subject in certain embodiments, a higher or more frequent dose of an anti-TNF treatment and an anti-IL-17 treatment may be needed. If these biomarkers have high abundance in a subject with an inflammatory disorder relative to a healthy subject in certain embodiments, a lower or less frequent dose of an anti-TNF treatment and an anti-IL-17 treatment may be needed.

In certain embodiments, CXCL1, CXCL2, CCL2, CXCL5, CXCL9, CXCL10, CCL23, TNF, IL-6, IL-22, IFNγ, CXCR4, GM-CSF, G-CSF and G-CSFR are decreased in abundance in subjects in response to administration of an anti-TNF treatment and an anti-IL-17 treatment. Thus, if these biomarkers have high abundance in a subject with an inflammatory disorder relative to a healthy subject in certain embodiments, a higher or more frequent dose of an anti-TNF treatment and an anti-IL-17 treatment may be needed. If these biomarkers have low abundance in a subject with an inflammatory disorder relative to a healthy subject in certain embodiments, a lower or less frequent dose of an anti-TNF treatment and an anti-IL-17 treatment may be needed.

An aspect of the invention provides a method of selecting a first subject suffering from an inflammatory disorder for treatment with a combination therapy comprising an anti-TNF treatment and an anti-IL-17 treatment comprising contacting a sample from the first subject with one or more binding moieties that specifically bind a protein or nucleic acid, for example LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof; detecting the interaction of the one or more binding moieties with the protein or nucleic acid, thereby detecting the relative abundance of the protein or nucleic acid in the first subject sample, comparing it to abundance of the protein or nucleic acid in a second subject sample, wherein the second subject does not suffer from the inflammatory disorder; and selecting the first subject for the combination therapy comprising an anti-TNF treatment and an anti-IL-17 treatment if the relative abundance of the protein or nucleic acid in the first subject sample is altered compared to the relative abundance of the protein or nucleic acid in the second subject sample. In an embodiment, the relative abundance of the protein or nucleic acid in the first subject sample is lower than the relative abundance of the protein or nucleic acid in the second subject sample. Alternatively, the relative abundance of the protein or nucleic acid in the first subject sample is lower than the relative abundance of the protein or nucleic acid in the second subject sample. In an embodiment, the relative abundance of the protein or nucleic acid in the first subject sample is compared to a post-treatment sample from the subject after anti-TNF treatment and an anti-IL-17 treatment of the subject or a cell sample from the subject. In various embodiments, the method comprises contacting a sample from a first subject with one or more binding moieties that specifically bind CXCL10 protein or nucleic acid; detecting the interaction of the one or more binding moieties with CXCL10 protein or nucleic acid, thereby detecting the relative abundance of CXCL10 protein or nucleic acid in the first subject sample, comparing the relative abundance of CXCL10 protein or nucleic acid to the relative abundance of CXCL10 protein or nucleic acid in a second subject sample, wherein the second subject does not suffer from the inflammatory disorder; and selecting the first subject for the combination therapy comprising an anti-TNF treatment and an anti-IL-17 treatment if the relative abundance of CXCL10 protein or nucleic acid in the first subject sample is higher than the relative abundance of CXCL10 protein or nucleic acid in the second subject sample.

In various embodiments, the method comprises contacting a sample from a first subject with one or more binding moieties that specifically bind CXCL1 protein or nucleic acid; detecting the interaction of the one or more binding moieties with CXCL1 protein or nucleic acid, thereby detecting the relative abundance of CXCL1 protein or nucleic acid in a first subject sample; comparing the relative abundance of the CLXCL1 protein or nucleic acid to the relative abundance of the CXCL1 protein or nucleic acid in a second subject sample, wherein the second subject does not suffer from the inflammatory disorder; and selecting the first subject for the combination therapy comprising an anti-TNF treatment and an anti-IL-17 treatment if the relative abundance of the CXCL1 protein or nucleic acid in the first subject sample is higher than the relative abundance of the CXCL1 protein or nucleic acid in the second subject sample.

In various embodiments, the method comprises contacting a sample from a first subject with one or more binding moieties that specifically bind G-CSF or G-CSFR; detecting the interaction of the one or more binding moieties with G-CSF or G-CSFR, thereby detecting the relative abundance of G-CSF or G-CSFR protein or nucleic acid in the first subject sample; comparing the relative abundance of G-CSF or G-CSFR protein or nucleic acid to the relative abundance of G-CSF or G-CSFR protein or nucleic acid in a second subject sample, wherein the second subject does not suffer from the inflammatory disorder; and selecting the first subject for the combination therapy comprising an anti-TNF treatment and an anti-IL-17 treatment if the relative abundance of G-CSF or G-CSFR protein or nucleic acid in the first subject sample is higher than the relative abundance of G-CSF or G-CDFR protein or nucleic acid in the second subject sample.

In various embodiments, the method comprises contacting a sample from a first subject with one or more binding moieties that specifically bind CXCR4; detecting the interaction of the one or more binding moieties with CXCR4, thereby detecting the relative abundance CXCR4 protein or nucleic acid in the first subject sample; comparing the relative abundance of CXCR4 protein or nucleic acid to the relative abundance of CXCR4 protein or nucleic acid in a second subject sample, wherein the second subject does not suffer from the inflammatory disorder; and selecting the first subject for the combination therapy comprising an anti-TNF treatment and an anti-IL-17 treatment if the relative abundance of CXCR4 protein or nucleic acid in the first subject sample is higher than the relative abundance of CXCR4 protein or nucleic acid in the second subject sample.

In various embodiments, the method comprises contacting a sample from a first subject with one or more binding moieties that specifically bind CXCR5; detecting the interaction of the one or more binding moieties with CXCR5, thereby detecting the relative abundance of the protein or nucleic acid in the first subject sample; comparing the relative abundance of the CXCR5 protein or nucleic acid to the relative abundance of the protein or nucleic acid in a second subject sample, wherein the second subject does not suffer from the inflammatory disorder; and selecting the subject for the combination therapy comprising an anti-TNF treatment and an anti-IL-17 treatment if the relative abundance of CXCR5 protein or nucleic acid in the first subject sample is lower than the relative abundance of CXCR5 protein or nucleic acid in the second subject sample.

In various embodiments, the method comprises contacting a sample from a first subject with one or more binding moieties that specifically bind GM-CSF or GM-CSFR; detecting the interaction of the one or more binding moieties with GM-CSF or GM-CSFR, thereby detecting the relative abundance of the protein or nucleic acid in the first subject sample; comparing the relative abundance of the GM-CSF or GM-CSFR protein or nucleic acid to the relative abundance of the protein or nucleic acid in a second subject sample, wherein the second subject does not suffer from the inflammatory disorder; and selecting the first subject for the combination therapy comprising an anti-TNF treatment and an anti-IL-17 treatment if the relative abundance of GM-CSF or GM-CSFR protein or nucleic acid in the first subject sample is higher than the relative abundance of GM-CSF or GM-CSFR protein or nucleic acid in the second subject sample.

In various embodiments, the method comprises contacting a sample from a first subject with one or more binding moieties that specifically bind IL-1Ra; detecting the interaction of the one or more binding moieties with IL-1Ra, thereby detecting the relative abundance of the protein or nucleic acid in the first subject sample; comparing the relative abundance of the IL-1Ra protein or nucleic acid to the relative abundance of the protein or nucleic acid in a second subject sample, wherein the second subject does not suffer from the inflammatory disorder; and selecting the first subject for the combination therapy comprising an anti-TNF treatment and an anti-IL-17 treatment if the relative abundance of IL-1Ra protein or nucleic acid in the first subject sample is lower than the relative abundance of IL-1Ra protein or nucleic acid in the second subject sample.

In various embodiments, the method comprises contacting a sample from a first subject with one or more binding moieties that specifically bind IL-10; detecting the interaction of the one or more binding moieties with IL-10, thereby detecting the relative abundance of the protein or nucleic acid in the first subject sample; comparing the relative abundance of the IL-10 protein or nucleic acid to the relative abundance of the protein or nucleic acid in a second subject sample, wherein the second subject does not suffer from the inflammatory disorder; and selecting the first subject for the combination therapy comprising an anti-TNF treatment and an anti-IL-17 treatment if the relative abundance of IL-10 protein or nucleic acid in the first subject sample is lower than the relative abundance of IL-10 protein or nucleic acid in the second subject sample.

In various embodiments, the method comprises contacting a sample from a first subject with one or more binding moieties that specifically bind TNF; detecting the interaction of the one or more binding moieties with TNF, thereby detecting the relative abundance of the protein or nucleic acid in the first subject sample; comparing the relative abundance of the TNF protein or nucleic acid to the relative abundance of the protein or nucleic acid in a second subject sample, wherein the second subject does not suffer from the inflammatory disorder; and selecting the first subject for the combination therapy comprising an anti-TNF treatment and an anti-IL-17 treatment if the relative abundance of TNF protein or nucleic acid in the first subject sample is higher than the relative abundance of TNF protein or nucleic acid in the second subject sample.

In various embodiments, the method comprises contacting a sample from a first subject with one or more binding moieties that specifically bind IFNγ; detecting the interaction of the one or more binding moieties with IFNγ, thereby detecting the relative abundance of the protein or nucleic acid in the first subject sample; comparing the relative abundance of the IFNγ protein or nucleic acid to the relative abundance of the protein or nucleic acid in a second subject sample, wherein the second subject does not suffer from the inflammatory disorder; and selecting the first subject for the combination therapy comprising an anti-TNF treatment and an anti-IL-17 treatment if the relative abundance of IFNγ protein or nucleic acid in the first subject sample is higher than the relative abundance of IFNγ protein or nucleic acid in the second subject sample.

In various embodiments, the method comprises contacting a sample from a first subject with one or more binding moieties that specifically bind IL-21; detecting the interaction of the one or more binding moieties with IL-21, thereby detecting the relative abundance of the protein or nucleic acid in the first subject sample; comparing the relative abundance of the IL-21 protein or nucleic acid to the relative abundance of the protein or nucleic acid in a second subject sample, wherein the second subject does not suffer from the inflammatory disorder; and selecting the first subject for the combination therapy comprising an anti-TNF treatment and an anti-IL-17 treatment if the relative abundance of IL-21 protein or nucleic acid in the first subject sample is lower than the relative abundance of IL-21 protein or nucleic acid in the second subject sample.

In various embodiments, the method comprises contacting a sample from a first subject with one or more binding moieties that specifically bind LIF; detecting the interaction of the one or more binding moieties LIF, thereby detecting the relative abundance of the protein or nucleic acid in the first subject sample; comparing the relative abundance of the LIF protein or nucleic acid to the relative abundance of the protein or nucleic acid in a second subject sample, wherein the second subject does not suffer from the inflammatory disorder; and selecting the first subject for the combination therapy comprising an anti-TNF treatment and an anti-IL-17 treatment if the relative abundance of LIF protein or nucleic acid in the first subject sample is lower than the relative abundance of LIF protein or nucleic acid in the second subject sample.

In various embodiments, the method comprises selecting the subject for the combination therapy comprising an anti-TNF treatment and an anti-IL-17 treatment if the relative abundance of the protein or nucleic acid in the subject sample is lower than the relative abundance of LIF protein or nucleic acid in the second subject sample.

In various embodiments, the method comprises selecting the subject for the combination therapy comprising an anti-TNF treatment and an anti-IL-17 treatment if the relative abundance of the protein or nucleic acid in the subject sample is higher than the relative abundance of LIF protein or nucleic acid in the second subject sample.

In various embodiments the binding moieties specifically bind to a homolog, derivative or portion of the target/biomarker molecule, e.g., LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof.

In various embodiments, the sample comprises cells, tissues or fluids obtained or isolated from a subject, as well as cells, tissues or fluids present within a subject. In various embodiments, the sample comprises a body fluid, tissue or a cell or collection of cells from a subject, as well as any component thereof, such as a fraction or an extract. In various embodiments, the tissue or cell is removed from the subject. In various embodiments, the tissue or cell is present within the subject. In various embodiments, the fluid comprises amniotic fluid, aqueous humor, vitreous humor, bile, blood, breast milk, cerebrospinal fluid, cerumen, chyle, cystic fluid, endolymph, feces, gastric acid, gastric juice, lymph, mucus, nipple aspirates, pericardial fluid, perilymph, peritoneal fluid, plasma, pleural fluid, pus, saliva, sebum, semen, sweat, serum, sputum, synovial fluid, tears, urine, vaginal secretions, or fluid collected from a biopsy. In one embodiment, the sample contains protein from the subject.

In another embodiment, the sample contains RNA (e.g., mRNA) from the subject or DNA (e.g., genomic DNA) from the subject. An aspect of the invention provides a method of determining whether a candidate substance is an effective treatment for an inflammatory disorder in a first subject in need thereof comprising contacting a sample from a second subject with the candidate substance, wherein the second subject suffers from the inflammatory disorder; wherein the candidate substance comprises one or more binding moieties that specifically bind LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof; detecting the interaction of the one or more binding moieties with LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof, thereby detecting the relative abundance of protein or nucleic acid in the sample; comparing the relative abundance of the protein or nucleic acid to the relative abundance of the protein or nucleic acid in a third subject sample, wherein the third subject suffers from the inflammatory disorder and the sample has not been contacted with the substance; and determining that the substance is an effective treatment for an inflammatory disorder in the first subject if the relative abundance of the protein or nucleic acid in the second subject sample is modulated (e.g., lower) than the relative abundance of the protein or nucleic acid in the third subject sample. Alternativley, determining that the substance is an effective treatment for an inflammatory disorder in the first subject if the relative abundance of the protein or nucleic acid in the second subject sample is higher than the relative abundance of the protein or nucleic acid in the third subject sample

In various embodiments, the method comprises contacting the sample with one or more binding moieties that specifically bind G-CSF or G-CSFR; detecting the interaction of the one or more binding moieties with G-CSF or G-CSFR, thereby detecting the relative abundance of G-CSF or G-CSFR protein or nucleic acid in the sample; comparing the relative abundance of G-CSF or G-CSFR protein or nucleic acid to the relative abundance of G-CSF or G-CSFR protein or nucleic acid in a third subject sample, wherein the third subject suffers from the inflammatory disorder and the sample has not been contacted with the substance; and determining that the substance is an effective treatment for an inflammatory disorder in the first subject if the relative abundance of G-CSF or G-CSFR protein or nucleic acid in the second subject sample is lower than the relative abundance of G-CSF or G-CSFR protein or nucleic acid in the third subject sample.

In various embodiments, the method further comprises contacting a sample from a second subject with the candidate substance, wherein the second subject suffers from the inflammatory disorder; contacting the sample with one or more binding moieties that specifically CXCL10; detecting the interaction of the one or more binding moieties with CXCL10, thereby detecting the relative abundance of CXCL10 protein or nucleic acid in the sample; comparing the relative abundance of the protein or nucleic acid to the relative abundance of the protein or nucleic acid in a third subject sample, wherein the third subject suffers from the inflammatory disorder and the sample has not been contacted with the substance; and determining that the substance is an effective treatment for an inflammatory disorder in the first subject if the relative abundance of CXCL10 protein or nucleic acid in the second subject sample is lower than the relative abundance of CXCL10 protein or nucleic acid in the third subject sample.

In various embodiments, the method further comprise contacting a sample from a second subject with the candidate substance, wherein the second subject suffers from the inflammatory disorder; contacting the sample with one or more binding moieties that specifically G-CSF; detecting the interaction of the one or more binding moieties with G-CSF, thereby detecting the relative abundance of G-CSF protein or nucleic acid in the sample, comparing the relative abundance of G-CSF protein or nucleic acid to the relative abundance of G-CSF protein or nucleic acid in a third subject sample, wherein the third subject suffers from the inflammatory disorder and the sample has not been contacted with the substance; and determining that the substance is an effective treatment for an inflammatory disorder in the first subject if the relative abundance of for example CXCL10 protein or nucleic acid, CXCL1 protein or nucleic acid and G-CSF protein or nucleic acid in the second subject sample is lower than the relative abundance of CXCL10 protein or nucleic acid, CXCL1 protein or nucleic acid and G-CSF protein or nucleic acid in the third subject sample.

In various embodiments, the method further comprises contacting a sample from a second subject with the candidate substance, wherein the second subject suffers from the inflammatory disorder; contacting the sample with one or more binding moieties that specifically CXCR4; detecting the interaction of the one or more binding moieties with CXCR4, thereby detecting the relative abundance of CXCR4 protein or nucleic acid in the sample; comparing the relative abundance of the CXCR4 protein or nucleic acid to the relative abundance of the CXCR4 protein or nucleic acid in a third subject sample, wherein the third subject suffers from the inflammatory disorder and the sample has not been contacted with the substance; and determining that the substance is an effective treatment for an inflammatory disorder in the first subject if the relative abundance of CXCR4 protein or nucleic acid in the second subject sample is lower than the relative abundance of CXCR4 protein or nucleic acid in the third subject sample.

In various embodiments, the method comprises contacting a sample from a second subject with the candidate substance, wherein the second subject suffers from the inflammatory disorder; contacting the sample with one or more binding moieties that specifically IFNγ; detecting the interaction of the one or more binding moieties with IFNγ, thereby detecting the relative abundance of the protein or nucleic acid in the sample; comparing the relative abundance of the IFNγ protein or nucleic acid to the relative abundance of the IFNγ protein or nucleic acid in a third subject sample, wherein the third subject suffers from the inflammatory disorder and the sample has not been contacted with the substance; and determining that the substance is an effective treatment for an inflammatory disorder in the first subject if the relative abundance of IFNγ protein or nucleic acid in the second subject sample is lower than the relative abundance of IFNγ protein or nucleic acid in the third subject sample.

In various embodiments, the method comprises contacting a sample from a second subject with the candidate substance, wherein the second subject suffers from the inflammatory disorder; contacting the sample with one or more binding moieties that specifically TNF; detecting the interaction of the one or more binding moieties with TNF, thereby detecting the relative abundance of the protein or nucleic acid in the sample; comparing the relative abundance of the TNF protein or nucleic acid to the relative abundance of the TNF protein or nucleic acid in a third subject sample, wherein the third subject suffers from the inflammatory disorder and the sample has not been contacted with the substance; and determining that the substance is an effective treatment for an inflammatory disorder in the first subject if the relative abundance of TNF protein or nucleic acid in the second subject sample is lower than the relative abundance of TNF protein or nucleic acid in the third subject sample.

In various embodiments, the method comprises contacting a sample from a second subject with the candidate substance, wherein the second subject suffers from the inflammatory disorder; contacting the sample with one or more binding moieties that specifically GM-CSF or GM-CSFR; detecting the interaction of the one or more binding moieties with GM-CSF or GM-CSFR, thereby detecting the relative abundance of the protein or nucleic acid in the sample; comparing the relative abundance of the GM-CSF or GM-CSFR protein or nucleic acid to the relative abundance of the GM-CSF or GM-CSFR protein or nucleic acid in a third subject sample, wherein the third subject suffers from the inflammatory disorder and the sample has not been contacted with the substance; and determining that the substance is an effective treatment for an inflammatory disorder in the first subject if the relative abundance of GM-CSF or GM-CSFR protein or nucleic acid in the second subject sample is lower than the relative abundance of GM-CSF or GM-CSFR protein or nucleic acid in the third subject sample.

In various embodiments, the method comprises contacting a sample from a second subject with the candidate substance, wherein the second subject suffers from the inflammatory disorder; contacting the sample with one or more binding moieties that specifically CXCR5; detecting the interaction of the one or more binding moieties with CXCR5, thereby detecting the relative abundance of the protein or nucleic acid in the sample, comparing the relative abundance of the CXCR5 protein or nucleic acid to the relative abundance of the CXCR5protein or nucleic acid in a third subject sample, wherein the third subject suffers from the inflammatory disorder and the sample has not been contacted with the substance; and determining that the substance is an effective treatment for an inflammatory disorder in the first subject if the relative abundance of CXCR5 protein or nucleic acid in the second subject sample is higher than the relative abundance of CXCR5 protein or nucleic acid in the third subject sample.

In various embodiments, the method comprises contacting a sample from a second subject with the candidate substance, wherein the second subject suffers from the inflammatory disorder; contacting the sample with one or more binding moieties that specifically IL-1Ra; detecting the interaction of the one or more binding moieties with IL-1Ra, thereby detecting the relative abundance of the protein or nucleic acid in the sample; comparing the relative abundance of the IL-1Ra protein or nucleic acid to the relative abundance of the IL-1Ra protein or nucleic acid in a third subject sample, wherein the third subject suffers from the inflammatory disorder and the sample has not been contacted with the substance; and determining that the substance is an effective treatment for an inflammatory disorder in the first subject if the relative abundance of IL-1Ra protein or nucleic acid in the second subject sample is higher than the relative abundance of IL-1Ra protein or nucleic acid in the third subject sample.

In various embodiments, the method comprises contacting a sample from a second subject with the candidate substance, wherein the second subject suffers from the inflammatory disorder; contacting the sample with one or more binding moieties that specifically IL-10; detecting the interaction of the one or more binding moieties with IL-10, thereby detecting the relative abundance of the protein or nucleic acid in the sample; comparing the relative abundance of the IL-10 protein or nucleic acid to the relative abundance of the IL-10 protein or nucleic acid in a third subject sample, wherein the third subject suffers from the inflammatory disorder and the sample has not been contacted with the substance; and determining that the substance is an effective treatment for an inflammatory disorder in the first subject if the relative abundance of IL-10 protein or nucleic acid in the second subject sample is higher than the relative abundance of IL-10 protein or nucleic acid in the third subject sample.

In various embodiments, the method comprises contacting a sample from a second subject with the candidate substance, wherein the second subject suffers from the inflammatory disorder; contacting the sample with one or more binding moieties that specifically IL-21; detecting the interaction of the one or more binding moieties with IL-21, thereby detecting the relative abundance of the protein or nucleic acid in the sample; comparing the relative abundance of the IL-21 protein or nucleic acid to the relative abundance of the IL-21 protein or nucleic acid in a third subject sample, wherein the third subject suffers from the inflammatory disorder and the sample has not been contacted with the substance; and determining that the substance is an effective treatment for an inflammatory disorder in the first subject if the relative abundance of IL-21 protein or nucleic acid in the second subject sample is higher than the relative abundance of IL-10 protein or nucleic acid in the third subject sample.

In various embodiments, the method comprises contacting a sample from a second subject with the candidate substance, wherein the second subject suffers from the inflammatory disorder; contacting the sample with one or more binding moieties that specifically LIF; detecting the interaction of the one or more binding moieties with LIF, thereby detecting the relative abundance of the protein or nucleic acid in the sample, comparing the relative abundance of the LIF protein or nucleic acid to the relative abundance of the LIF protein or nucleic acid in a third subject sample, wherein the third subject suffers from the inflammatory disorder and the sample has not been contacted with the substance; and determining that the substance is an effective treatment for an inflammatory disorder in the first subject if the relative abundance of LIF protein or nucleic acid in the second subject sample is higher than the relative abundance of LIF protein or nucleic acid in the third subject sample.

An aspect of the invention provides a method of determining whether a with a combination therapy comprising an anti-TNF treatment and an anti-IL-17 treatment is an effective treatment for an inflammatory disorder in a first subject in need thereof comprising contacting a sample from a second subject with the combination therapy, wherein the second subject suffers from the inflammatory disorder; contacting the sample with one or more binding moieties that specifically bind at least one protein selected from the group consisting of: LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof; detecting the interaction of the one or more binding moieties with the at least one protein, thereby detecting the relative abundance of the protein or nucleic acid in the sample; comparing the relative abundance of CXCL10 protein or nucleic acid to the relative abundance of the protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of the protein or nucleic acid in the second subject sample is lower than the relative abundance of the protein or nucleic acid in the third subject sample.

In various embodiments, the method comprises contacting a sample from a second subject with the combination therapy, wherein the second subject suffers from the inflammatory disorder; contacting the sample with one or more binding moieties that specifically bind CXCL1; detecting the interaction of the one or more binding moieties with CXCL1, thereby detecting the relative abundance of the protein or nucleic acid in the sample, comparing the relative abundance of the CXCL1 protein or nucleic acid to the relative abundance of the protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of the CXCL1 protein or nucleic acid in the second subject sample is lower than the relative abundance of the CXCL1 protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind G-CSF or G-CSFR; detecting the interaction of the one or more binding moieties with G-CSF or G-CSFR, thereby detecting the relative abundance of G-CSF or G-CSFR protein or nucleic acid in the sample, comparing the relative abundance of G-CSF or G-CSFR protein or nucleic acid to the relative abundance of G-CSF or G-CSFR protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of G-CSF or G-CSFR protein or nucleic acid in the second subject sample is lower than the relative abundance of G-CSF or G-CSFR protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind CXCL10; detecting the interaction of the one or more binding moieties with CXCL10, thereby detecting the relative abundance of CXCL10 protein or nucleic acid in the sample, comparing the relative abundance of the protein or nucleic acid to the relative abundance of the protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of CXCL10 protein or nucleic acid in the second subject sample is lower than the relative abundance of CXCL10 protein or nucleic acid the in third subject sample.

In various embodiments, the method further comprises contacting a sample from a second subject with the combination therapy, wherein the second subject suffers from the inflammatory disorder; contacting the sample with one or more binding moieties that specifically bind G-CSF; detecting the interaction of the one or more binding moieties with G-CSF, thereby detecting the relative abundance of G-CSF protein or nucleic acid in the sample, comparing the relative abundance of G-CSF protein or nucleic acid to the relative abundance of G-CSF protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of for example CXCL10 protein or nucleic acid, CXCL1 protein or nucleic acid and G-CSF protein or nucleic acid in the second subject sample is lower than the relative abundance of CXCL10 protein or nucleic acid, CXCL1 protein or nucleic acid and G-CSF protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind CXCR4; detecting the interaction of the one or more binding moieties with CXCR4, thereby detecting the relative abundance of CXCR4 protein or nucleic acid in the sample, comparing the relative abundance of the protein or nucleic acid to the relative abundance of the protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of CXCR4 protein or nucleic acid in the second subject sample is lower than the relative abundance of CXCR4 protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind IFNγ; detecting the interaction of the one or more binding moieties with IFNγ, thereby detecting the relative abundance of the protein or nucleic acid in the sample; comparing the relative abundance of the IFNγ protein or nucleic acid to the relative abundance of the IFNγ protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of IFNγ protein or nucleic acid in the second subject sample is lower than the relative abundance of IFNγ protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind TNF; detecting the interaction of the one or more binding moieties with TNF, thereby detecting the relative abundance of the protein or nucleic acid in the sample; comparing the relative abundance of the TNF protein or nucleic acid to the relative abundance of the TNF protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of TNF protein or nucleic acid in the second subject sample is lower than the relative abundance of TNF protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind GM-CSF or GM-CSFR; detecting the interaction of the one or more binding moieties with GM-CSF or GM-CSFR, thereby detecting the relative abundance of the protein or nucleic acid in the sample, comparing the relative abundance of the GM-CSF or GM-CSFR protein or nucleic acid to the relative abundance of the GM-CSF or GM-CSFR protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of GM-CSF or GM-CSFR protein or nucleic acid in the second subject sample is lower than the relative abundance of GM-CSF or GM-CSFR protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind CXCR5; detecting the interaction of the one or more binding moieties with CXCR5, thereby detecting the relative abundance of the protein or nucleic acid in the sample; comparing the relative abundance of the CXCR5 protein or nucleic acid to the relative abundance of the CXCR5 protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of CXCR5 protein or nucleic acid in the second subject sample is higher than the relative abundance of CXCR5 protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind IL-1Ra detecting the interaction of the one or more binding moieties with IL-1Ra, thereby detecting the relative abundance of the protein or nucleic acid in the sample, comparing the relative abundance of the IL-1Ra protein or nucleic acid to the relative abundance of the IL-1Ra protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of IL-1Ra protein or nucleic acid in the second subject sample is higher than the relative abundance of IL-1Ra protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind IL-1Ra; detecting the interaction of the one or more binding moieties with IL-10, thereby detecting the relative abundance of the protein or nucleic acid in the sample, comparing the relative abundance of the IL-10 protein or nucleic acid to the relative abundance of the IL-10 protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of IL-10 protein or nucleic acid in the second subject sample is higher than the relative abundance of IL-10 protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind IL-21; detecting the interaction of the one or more binding moieties with IL-21, thereby detecting the relative abundance of the protein or nucleic acid in the sample; comparing the relative abundance of the IL-21 protein or nucleic acid to the relative abundance of the IL-21 protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of IL-21 protein or nucleic acid in the second subject sample is higher than the relative abundance of IL-21 protein or nucleic acid the in third subject sample.

In various embodiments, the method comprises contacting a sample from a second subject with the combination therapy, wherein the second subject suffers from the inflammatory disorder; contacting the sample with one or more binding moieties that specifically bind LIF; detecting the interaction of the one or more binding moieties with LIF, thereby detecting the relative abundance of the protein or nucleic acid in the sample, comparing the relative abundance of the LIF protein or nucleic acid to the relative abundance of the LIF protein or nucleic acid in a third subject sample, wherein the third subject suffers from the inflammatory disorder and the sample has not been contacted with the combination therapy; and determining that the combination therapy is an effective treatment for an inflammatory disorder in the first subject if the relative abundance of LIF protein or nucleic acid in the second subject sample is higher than the relative abundance of IL-21 protein or nucleic acid the in third subject sample.

In various embodiments, the method comprises selecting the subject for the combination therapy comprising an anti-TNF treatment and an anti-IL-17 treatment, or determining whether a candidate substance is an effective treatment for an inflammatory disorder occurs if the relative abundance of the protein or nucleic acid in the subject sample is lower than the relative abundance of LIF protein or nucleic acid in the second subject sample.

In various embodiments, the method comprises selecting the subject for the combination therapy comprising an anti-TNF treatment and an anti-IL-17 treatment or determining whether a candidate substance is an effective treatment for an inflammatory disorder occurs if the relative abundance of the protein or nucleic acid in the subject sample is higher than the relative abundance of LIF protein or nucleic acid in the second subject sample.

In various embodiments of the method, the anti-TNF treatment comprises an anti-TNF binding protein. For example, the anti-TNF treatment includes anti-TNFα treatment. In various embodiments of the method, the anti-TNF binding protein comprises a fusion protein, an antibody, or antigen binding fragment thereof, that specifically binds to TNF. In various embodiments, the anti-TNF binding protein comprises an antibody, or antigen binding fragment thereof, and is a murine antibody, a human antibody, a humanized antibody, a bispecific antibody, a chimeric antibody, a Fab, a Fab′, a F(ab′)2, an ScFv, an SMIP, an affibody, an avimer, a versabody, a nanobody, a domain antibody, or an antigen binding fragment thereof.

In various embodiments of the method, the anti-TNF antibody comprises a human anti-TNF antibody. In various embodiments of the method, the human anti-TNFα antibody comprises Adalimumab, or an antigen binding fragment thereof. In various embodiments of the method, the anti-TNF antibody comprises a humanized anti-TNF antibody. For example, the humanized anti-TNF antibody comprises infliximab, or an antigen binding fragment thereof. In various embodiments of the method, the anti-TNF binding protein comprises an anti-TNFα fusion protein. For example, the anti-TNFα binding protein comprises etanercept, or an antigen binding fragment thereof.

In various embodiments of the method, the anti-IL-17 treatment comprises an anti-IL-17 binding protein. In various embodiments of the method, the anti-IL-17 binding protein comprises a fusion protein, an antibody, or antigen binding fragment thereof, that specifically binds to IL-17. For example, the anti-IL-17 binding protein comprises an antibody, or antigen binding fragment thereof, and is a murine antibody, a human antibody, a humanized antibody, a bispecific antibody, a chimeric antibody, a Fab, a Fab′, a F(ab′)2, an ScFv, an SMIP, an affibody, an avimer, a versabody, a nanobody, a domain antibody, or an antigen binding fragment thereof.

In various embodiments of the method, the anti-IL-17 antibody comprises a humanized antibody. For example, the anti-IL-17 antibody is ixekizumab, 10F7, B6-17, or an antigen binding fragment thereof.

In various embodiments of the method, n the anti-IL-17 binding protein comprises a fusion protein, an antibody, or antigen binding fragment thereof, that specifically binds to IL-17.

In various embodiments of the method, the anti-IL-17 treatment comprises methotrexate, an analog thereof, or a pharmaceutically acceptable salt thereof.

The combination treatment in various embodiments of the method further comprises methotrexate, an analog thereof, or a pharmaceutically acceptable salt thereof. In various embodiments of the method, the combination therapy comprises the administration of a multispecific binding protein that binds at least one of TNF and IL-17. For example, the multispecific binding protein is selected from the group consisting of a dual variable domain immunoglobulin (DVD-Ig) molecule, a half-body DVD-Ig (hDVD-Ig) molecule, a triple variable domain immunoglobulin (TVD-Ig) molecule, a receptor variable domain immunoglobulin (rDVD-Ig) molecule, a polyvalent DVD-Ig (pDVD-Ig) molecule, a monobody DVD-Ig (mDVD-Ig) molecule, a cross over (coDVD-Ig) molecule, a blood brain barrier (bbbDVD-Ig) molecule, a cleavable linker DVD-Ig (clDVD-Ig) molecule, and a redirected cytotoxicity DVD-Ig (rcDVD-Ig) molecule. In various embodiments of the method, the multispecific binding protein binds TNFα and IL-17. For example, the binding protein comprises a DVD-Ig protein in Table 4, Table 6 or Table 7. In various embodiments, the DVD-Ig protein comprises at least one variable heavy chain domain selected from Table 4, Table 6 and Table 7. In various embodiments, the DVD-Ig protein comprises at least one variable heavy chain domain selected from the group consisting of: SEQ ID NO: 5, SEQ ID NO: 11, and SEQ ID NO: 21. In various embodiments, the DVD-Ig protein comprises at least one variable light chain domain selected from Table 4, Table 6 and Table 7. In various embodiments, the DVD-Ig protein comprises at least one variable light chain domain selected from the group consisting of: SEQ ID NO: 8, SEQ ID NO: 16, and SEQ ID NO: 26. In various embodiments, the combination therapy comprises a multispecific binding protein that binds TNF and IL-17 and comprises at least one of: a heavy chain amino acid sequence selected from SEQ ID NOs: 5, 11 and 24; a light chain amino acid sequence selected from SEQ ID NOs: 8, 16, and 26; a heavy chain constant region selected from SEQ ID NOs: 7, 15, and 25; or a light chain constant region selected from SEQ ID NOs: 10, 20 and 30.

In various embodiments of the method, the one or more binding moieties specifically bind nucleic acids. In various embodiments of the method, the one or more binding moieties specifically bind RNA. In various embodiments of the method, the one or more binding moieties specifically bind mRNA, miRNA, or hnRNA. In various embodiments of the method, the one or more binding moieties specifically bind DNA. In various embodiments of the method, the one or more binding moieties F specifically bind cDNA.

In various embodiments of the method, the one or more binding moieties are appropriate for use in a technique selected from the group consisting of a polymerase chain reaction (PCR) amplification reaction, reverse-transcriptase PCR analysis, quantitative reverse-transcriptase PCR analysis, Northern blot analysis, an RNAase protection assay, digital RNA detection/quantitation, and a combination or sub-combination thereof.

In various embodiments of the method, the one or more binding moieties specifically bind protein. In various embodiments of the method, the one or more binding moieties are binding proteins that bind at least one of LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof. For example, the one or more binding proteins comprise an antibody, or antigen binding fragment thereof, that specifically binds to the protein. In various embodiments of the method, the antibody or antigen binding fragment thereof is selected from the group consisting of a murine antibody, a human antibody, a humanized antibody, a bispecific antibody, a chimeric antibody, a Fab, a Fab′, a F(ab′)₂, an scFv, an SMIP, an affibody, an avimer, a versabody, a nanobody, a domain antibody, and an antigen binding fragment thereof.

In various embodiments of the method, the one or more binding proteins comprise a multispecific binding protein. For example, the multispecific binding protein is selected from the group consisting of a DVD-Ig molecule, a hDVD-Ig molecule, a TVD-Ig molecule, a rDVD-Ig molecule, a pDVD-Ig molecule, amDVD-Ig molecule, a coDVD-Ig molecule, a bbbDVD-Ig molecule, a clDVD-Ig molecule, and a rcDVD-Ig molecule.

In various embodiments of the method, the binding protein comprises a label. For example, the label is selected from the group consisting of a radio-label, a biotin-label, a chromophore, a fluorophore, and an enzyme. In various embodiments of the method, the one or more binding moieties are appropriate for use with a technique selected from the group consisting of an immunoassay, a western blot analysis, a radioimmunoassay, immunofluorimetry, immunoprecipitation, equilibrium dialysis, immunodiffusion, an electrochemiluminescence immunoassay (ECLIA), an ELISA assay, a polymerase chain reaction, an immunopolymerase chain reaction, and combinations or sub-combinations thereof. The immunoassay for example comprises a solution-based immunoassay selected from the group consisting of electrochemiluminescence, chemiluminescence, fluorogenic chemiluminescence, fluorescence polarization, and time-resolved fluorescence. In various embodiments, the immunoassay comprises a sandwich immunoassay selected from the group consisting of electrochemiluminescence, chemiluminescence, and fluorogenic chemiluminescence.

In various embodiments of the method, any of the samples from the subjects comprises a fluid, or component thereof, obtained from any of the subjects. In various embodiments of the method, the fluid is selected from the group consisting of blood, serum, synovial fluid, lymph, plasma, urine, amniotic fluid, aqueous humor, vitreous humor, bile, breast milk, cerebrospinal fluid, cerumen, chyle, cystic fluid, endolymph, feces, gastric acid, gastric juice, mucus, nipple aspirates, pericardial fluid, perilymph, peritoneal fluid, pleural fluid, pus, saliva, sebum, semen, sweat, serum, sputum, tears, vaginal secretions, and fluid collected from a biopsy. In various embodiments of the method, any of the samples from the subjects comprises a tissue or cell, or component thereof, obtained from any of the subjects.

In various embodiments of the method, any of the subjects is a mammalian subject. For example, the mammal is selected from the group consisting of a human, a mouse, a rat, a non-human primate, a dog, a cat, a rabbit, a sheep, a goat and a pig. In various embodiments of the method, the mammal is a human.

The inflammatory disorder in various embodiments of the method is selected from the group consisting of arthritis; necrotizing enterocolitis (NEC); gastroenteritis; intestinal flu; stomach flu; pelvic inflammatory disease (PID); emphysema; pleurisy; pyelitis; pharyngitis; sore throat; angina; acne vulgaris; rubor; urinary tract infection; appendicitis; bursitis; colitis; cystitis; dermatitis; phlebitis; rhinitis; tendonitis; tonsillitis; vasculitis; asthma; autoimmune diseases; celiac disease; chronic prostatitis; glomerulonephritis; hypersensitivities; inflammatory bowel diseases; pelvic inflammatory disease; reperfusion injury; sarcoidosis; transplant rejection; vasculitis; interstitial cystitis; hay fever; periodontitis; atherosclerosis; psoriasis; ankylosing spondylitis; juvenile idiopathic arthritis; Behcet's disease; spondyloarthritis; uveitis; systemic lupus erythematosus, and cancer. For example, the arthritis includes rheumatoid arthritis, psoriatic arthritis, osteoarthritis or juvenile idiopathic arthritis. In various embodiments, the inflammatory disease is rheumatoid arthritis and the subject is being treated with at least one additional therapeutic agent, e.g., a protein, small molecule, and polynucleotide. For example, the therapeutic agent comprises a DMARD. In certain embodiments, the combination therapy for the TNF antibody and IL-17 antibody is administered concurrently or subsequently with the therapeutic agent. In various embodiments of the method, the therapeutic agent is a DMARD, for example the DMARD comprises a biologic or a compound (e.g., a small molecule). In various embodiments, the DMARD comprises methotrexate, sulfasalazine, cyclosporine, leflunomide, hydroxychloroquine, or zathioprine. In various embodiments, the combination therapy and DMARD are administered concurrently. Alternatively, the combination therapy and DMARD are administered at different times (i.e., at a time prior to or after administering the other).

In various embodiments, the combination therapy comprises a therapeutically effective amount, e.g., specific dose of a binding protein that binds both TNF and IL-17, and a combination of binding proteins in which one binds TNF and at least one other binding protein binds IL-17. In various embodiments, the binding protein comprises a modulator or inhibitor of TNF and/or IL-17. In various embodiments, the binding protein specifically binds at least one epitope of TNF and/or IL-17.

An aspect of the invention provides a kit comprising a binding moiety that specifically binds to a LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof. In various embodiments of the kit, the one or more binding moieties that specifically bind LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR nucleic acids. In various embodiments, the one or more binding moieties specifically bind nucleic acids. In various embodimetns, the one or more binding moieties specifically bind RNA. In various embodiments of the kit, the one or more binding moieties specifically bind mRNA, miRNA, or hnRNA. In various embodiments of the kit, the one or more binding moieties specifically bind DNA. In various embodiments of the kit, the one or more binding moieties that specifically bind cDNA. In various embodiments, the one or more binding moieties specifically bind cDNA.

The one or more binding moieties in various embodiments of the kit are appropriate for use in a technique selected from the group consisting of a polymerase chain reaction (PCR) amplification reaction, reverse-transcriptase PCR analysis, quantitative reverse-transcriptase PCR analysis, Northern blot analysis, an RNAase protection assay, digital RNA detection/quantitation, and a combination or sub-combination thereof.

In various embodiments of the kit, the one or more binding moieties specifically bind protein.

In various embodiments of the kit, the one or more binding moieties are binding proteins that bind at least one of LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof. For example, the one or more binding proteins comprise an antibody, or antigen binding fragment thereof, that specifically binds to the protein. In various embodiments of the kit, the antibody or antigen binding fragment thereof is selected from the group consisting of a murine antibody, a human antibody, a humanized antibody, a bispecific antibody, a chimeric antibody, a Fab, a Fab′, a F(ab′)₂, an scFv, an SMIP, an affibody, an avimer, a versabody, a nanobody, a domain antibody, and an antigen binding fragment thereof.

In various embodiments of the kit, the one or more binding proteins comprise a multispecific binding protein. For example, the multispecific binding protein is selected from the group consisting of a DVD-Ig molecule, a hDVD-Ig molecule, a TVD-Ig molecule, a rDVD-Ig molecule, a pDVD-Ig molecule, a mDVD-Ig molecule, a coDVD-Ig molecule, a bbbDVD-Ig molecule, a clDVD-Ig molecule, and a rcDVD-Ig molecule.

In various embodiments of the kit, the binding protein comprises a label. For example, the label is selected from the group consisting of a radio-label, a biotin-label, a chromophore, a fluorophore, and an enzyme.

In various embodiments of the kit, the one or more binding moieties that specifically bind LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof are appropriate for use with a technique selected from the group consisting of an immunoassay, a western blot analysis, a radioimmunoassay, immunofluorimetry, immunoprecipitation, equilibrium dialysis, immunodiffusion, an electrochemiluminescence immunoassay (ECLIA), an ELISA assay, a polymerase chain reaction, an immunopolymerase chain reaction, and combinations or sub-combinations thereof.

For example, the immunoassay comprises a solution-based immunoassay selected from the group consisting of electrochemiluminescence, chemiluminescence, fluorogenic chemiluminescence, fluorescence polarization, and time-resolved fluorescence. In various embodiments of the kit, the immunoassay comprises a sandwich immunoassay selected from the group consisting of electrochemiluminescence, chemiluminescence, and fluorogenic chemiluminescence.

An aspect of the invention provides a method of determining effectivess of a combination therapy comprising an anti-TNF treatment and an anti-IL-17 treatment, and/or a selecting a subject suffering from an inflammatory disorder for treatment with the combination therapy, the method comprising: contacting a sample from the subject with one or more binding moieties that specifically bind a protein or a nucleic acid encoding the protein, wherein the protein is selected from the group consisting of: LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof; detecting the interaction of the one or more binding moieties with the protein or nucleic acid, thereby detecting the relative abundance or expression of the protein or nucleic acid in the sample, comparing the relative abundance or expression of the protein or nucleic acid to the relative abundance or expression of the protein or nucleic acid in a second subject sample, wherein the second subject does not suffer from the inflammatory disorder; and selecting the subject for the combination therapy comprising an anti-TNF treatment and an anti-IL-17 treatment if the relative abundance or expression ofthe protein or nucleic acid in the subject sample is modulated compared to the relative abundance or expression of the protein or nucleic acid in the second subject sample.

A related aspect of the invention provides a method of determining effectivess of a combination therapy comprising an anti-TNF treatment and an anti-IL-17 treatment for a subject receiving the therapy, the method comprising contacting a sample from the subject with one or more binding moieties that specifically bind a protein or a nucleic acid encoding the protein, wherein the protein is selected from the group consisting of: LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof; detecting the interaction of the one or more binding moieties with the protein or nucleic acid and/or expression on a cell surface of cells in the sample, thereby detecting the relative abundance or expression of the protein or nucleic acid in the sample, comparing the relative abundance or expression of the protein or nucleic acid to the relative abundance of the protein or nucleic acid in a control sample, wherein the control sample is from the subject prior to having received the combination therapy; and selecting the subject for the combination therapy comprising an anti-TNF treatment and an anti-IL-17 treatment if the relative abundance or expression of the protein or nucleic acid in the subject sample is modulated compared to the relative abundance or expression of the protein or nucleic acid in the second subject sample.

In various embodiments of the method, the protein is CXCL10, and selecting the combination therapy occurs if the abundance or expression of protein or nucleic acid in the subject sample is higher compared to than the relative abundance or expression of CXCL10 the protein or nucleic acid in the second subject sample. In various embodiments, the protein is CXCL1, and selecting the combination therapy occurs if the relative abundance or expression of CXCL1 protein or nucleic acid in the subject sample is higher than the relative abundance or expression of CXCL1 protein or nucleic acid in the second subject sample.

In various embodiments, the protein is G-CSF or G-CSFR, and selecting the combination therapy occurs if the relative abundance or expression of the G-CSF or G-CSFR protein or nucleic acid in the subject sample is higher than the relative abundance or expression of the G-CSF or G-CSFR protein or nucleic acid in the second subject sample.

In various embodiments of the method, the protein is IL-1Ra, and selecting the combination therapy occurs if the relative abundance or expression of the IL-1Ra protein or nucleic acid in the subject sample is higher than the relative abundance or expression of the IL-1Ra protein or nucleic acid in the second subject sample.

In various embodiments, the protein is IFNγ, and selecting the combination therapy occurs if the relative abundance or expression of IFNγ protein or nucleic acid in the subject sample similar or is higher than the relative abundance or expression of IFNγ protein or nucleic acid in the second subject sample.

In various embodiments, the protein is IL-21, and selecting the combination therapy occurs if the relative abundance or expression of IL-21 protein or nucleic acid in the subject sample is higher than the relative abundance or expression of IL-21 protein or nucleic acid in the second subject sample.

In various embodiments, the protein is LIF, and selecting the combination therapy occurs if the relative abundance or expression of LIF protein or nucleic acid in the subject sample is about the same or higher than the relative abundance or expression of LIF protein or nucleic acid in the second subject sample.

In various embodiments, the protein is GM-CSF or GM-CSFR, and selecting the combination therapy occurs if the relative abundance or expression of GM-CSF or GM-CSFR protein or nucleic acid in the subject sample is the same or lower than the relative abundance or expression of G-CSF or GM-CSFR protein or nucleic acid in the second subject sample.

In various embodiments, the protein is CXCR5, and selecting the combination therapy occurs if the relative abundance or expression of CXCR5 protein or nucleic acid in the subject sample is higher than the relative abundance or expression of GCXCR5 protein or nucleic acid in the second subject sample. In various embodiments, the protein is CXCR4, and selecting the combination therapy occurs if the relative abundance or expression of CXCR4 protein or nucleic acid in the subject sample is lower than the relative abundance or expression of GCXCR4 protein or nucleic acid in the second subject sample.

In various embodiments, the protein is IL-10, and selecting the combination therapy occurs if the relative abundance or expression of IL-10 protein or nucleic acid in the subject sample is higher than the relative abundance or expression of IL-10 protein or nucleic acid in the second subject sample.

In various embodiments, the protein is TNF, and selecting the combination therapy occurs if the relative abundance or expression of TNF protein or nucleic acid in the subject sample is lower than the relative abundance or expression of TNF protein or nucleic acid in the second subject sample.

In various embodiments, the relative abundance or expression of theTNF protein or nucleic acid in the subject sample is lower than the relative abundance or expression of TNF protein or nucleic acid in the second subject sample.

In various embodiments, the method further comprises stimulating the sample. For example, stimulating the sample comprises using at least one substance selected from the group consisting of: lipopolysaccharide, CD3, and CD28.

In various embodiments, the one or more binding proteins comprise an antibody, or antigen binding fragment thereof, that specifically binds to the protein. For example, the antibody or antigen binding fragment thereof is selected from the group consisting of a murine antibody, a human antibody, a humanized antibody, a bispecific antibody, a chimeric antibody, a Fab, a Fab′, a F(ab′)₂, an scFv, an SMIP, an affibody, an avimer, a versabody, a nanobody, a domain antibody, and an antigen binding fragment thereof.

In various embodiments, the one or more binding proteins comprise a multispecific binding protein. For example, the multispecific binding protein is selected from the group consisting of a DVD-Ig molecule, a hDVD-Ig molecule, a TVD-Ig molecule, a rDVD-Ig molecule, a pDVD-Ig molecule, a mDVD-Ig molecule, a coDVD-Ig molecule, a bbbDVD-Ig molecule, a clDVD-Ig molecule, and a rcDVD-Ig molecule.

In various embodiments, the the multispecific binding protein comprises a DVD-Ig protein. In various embodiments, the DVD-Ig binding protein comprises at least one variable heavy chain domain or CDR selected from Table 1, Table 4, Table 6 and Table 7.

In various embodiments, wherein the binding protein comprises the variable heavy (VH) complementarity determining regions (CDRs) for binding TNFα from the amino acid sequence of SEQ ID NO: 12 and/or the the VH CDRs for binding IL-17 from the amino acid sequence of SEQ ID NO: 14.

In various embodiments, the binding protein comprises the VL CDRs for binding TNFα from the amino acid sequence of SEQ ID NO: 17 and/or the VL CDRs for binding IL-17 from the amino acid sequence of SEQ ID NO: 19.

In various embodiments, the binding protein comprises the variable heavy (VH) complementarity determining regions (CDRs) for binding TNFα from the amino acid sequence of SEQ ID NO: 22 and/or the the VH CDRs for binding IL-17 from the amino acid sequence of SEQ ID NO: 24.

In various embodiments, the binding protein comprises the VL CDRs for binding TNFα from the amino acid sequence of SEQ ID NO: 27 and/or the VL CDRs for binding IL-17 from the amino acid sequence of SEQ ID NO: 29.

In various embodiments, the binding protein further comprises a constant region. For example, the constant region is found in Tables 4, Table 6, and Table 7. In various embodiments, the CH region is selected from the amino acid sequence of SEQ ID NOs: 7, 15 and 25. In various embodiments, the CL region is selected from the amino acid sequence of SEQ ID NOs: 10, 20 and 30.

In various embodiments, the binding protein comprises a label. For example, the label is selected from the group consisting of a radio-label, a biotin-label, a chromophore, a fluorophore, and an enzyme.

In various embodiments of the method, detecting comprises using at least a technique selected from the group consisting of an immunoassay, a western blot analysis, a radioimmunoassay, immunofluorimetry, immunoprecipitation, equilibrium dialysis, immunodiffusion, an electrochemiluminescence immunoassay (ECLIA), an ELISA assay, a polymerase chain reaction, an immunopolymerase chain reaction, and

In various embodiments, the sample comprises a suspension, a fluid, or component thereof, obtained from any of the subjects. In various embodiments, the samples comprises a plurality of cells. For example, the cells are T cells, B cells, or monocytes. In various embodiments, the fluid is selected from the group consisting of blood, serum, synovial fluid, lymph, plasma, urine, amniotic fluid, aqueous humor, vitreous humor, bile, breast milk, cerebrospinal fluid, cerumen, chyle, cystic fluid, endolymph, feces, gastric acid, gastric juice, mucus, nipple aspirates, pericardial fluid, perilymph, peritoneal fluid, pleural fluid, pus, saliva, sebum, semen, sweat, serum, sputum, tears, vaginal secretions, and fluid collected from a biopsy.

The description provides a method of monitoring or calibrating a dosage in a subject being treated for an inflammatory disorder with a combination therapy comprising an anti-TNF treatment and an anti-IL-17 treatment, the method comprising the steps of administering to the subject a first dose of a combination therapy comprising an anti-TNF treatment and an anti-IL-17 treatment; determining a modulation of expression of one or more biomarkers in a sample from the subject, wherein the one or more biomarkers are gene products selected from the group consisting of LIF, CXCL1, CXCL2, CXCL5, CXCL9, CXCL10, CCL2, CCL23, IL-1Ra, TNF, IL-6, IL-10, IL-21, IL-22, IFNγ, CXCR4, CXCR5, GM-CSF, G-CSF and G-CSFR; detecting the interaction of one or more binding moieties that specifically bind to the one or more biomarkers, thereby detecting the abundance of the one or more biomarkers in the subject sample; and obtaining a relative abundance of the one or more biomarkers in the subject sample by comparison to a baseline abundance of the biomarker; administering a second dose of the combination therapy, wherein the second dose is determined depending on the relative abundance of the one or more biomarkers in the subject sample in response to the first dose.

In various embodiments, the second dose is equal to or greater than the first dose when the one or more biomarkers are gene products selected from the group consisting of LIF, IL-1RA, IL-10, IL-21 and CXCR5, and wherein the relative abundance of the one or more biomarkers in the subject sample in response to the first dose compared to the baseline abundance of the one or more biomarker is less. In various embodiments, the second dose is equal to or greater than the first dose when the one or more biomarkers are gene products selected from the group consisting of CXCL1, CXCL2, CCL2, CXCL5, CXCL9, CXCL10, CCL23, TNF, IL-6, IL-22, IFNγ, CXCR4, GM-CSF, G-CSF and G-CSFR, and wherein the relative abundance of the one or more biomarkers in the subject sample in response to the first dose compared to the baseline abundance of the one or more biomarker is greater. In various embodiments, the second dose is less than the first dose or treatment is discontinued when one or more biomarkers are gene products selected from the group consisting of LIF, IL-1RA, IL-10, IL-21 and CXCR5, and wherein the relative abundance of the one ore mores biomarker in the subject sample in response to the first dose compared to the baseline abundance of the one or more biomarker is greater. In various embodiments, the second dose is less than the first dose or treatment is discontinued when one or more biomarkers are gene products selected from the group consisting of CXCL1, CXCL2, CCL2, CXCL5, CXCL9, CXCL10, CCL23, TNF, IL-6, IL-22, IFNγ, CXCR4, GM-CSF, G-CSF and G-CSFR and wherein the relative abundance of the one or more biomarkers in the subject sample in response to the first dose compared to the baseline abundance of the one or more biomarker is less.

The description also provides a method of treating a subject suffering from an inflammatory disorder, the method comprising the steps of administering a dose of a combination therapy comprising an anti-TNF treatment and an anti-IL-17 treatment to the subject, wherein a sample from the subject comprises an abundance of one or more biomarkers, wherein the one or more biomarkers are gene products selected from the group consisting of LIF, IL-1RA, IL-10, IL-21 and CXCR5, and wherein the relative abundance of the one or more biomarkers in the subject sample compared to a baseline abundance of the one or more markers is less.

The description also provides a method of treating a subject suffering from an inflammatory disorder, the method comprising the steps of administering a dose of a combination therapy comprising an anti-TNF treatment and an anti-IL-17 treatment to the subject, wherein a sample from the subject comprises an abundance of one or more biomarkers, wherein the one or more biomarkers are gene products selected from the group consisting of CXCL1, CXCL2, CCL2, CXCL5, CXCL9, CXCL10, CCL23, TNF, IL-6, IL-22, IFNγ, CXCR4, GM-CSF, G-CSF and G-CSFR, and wherein the relative abundance of the one or more biomarkers in the subject sample compared to a baseline abundance of the one or more markers is less.

The description also provides a method of treating a subject suffering from an inflammatory disorder, the method comprising the steps of determining a modulation of expression of one or more biomarkers in a sample from the subject, wherein the one or more biomarkers are gene products selected from the group consisting of LIF, CXCL1, CXCL2, CXCL5, CXCL9, CXCL10, CCL2, CCL23, IL-1Ra, TNF, IL-6, IL-10, IL-21, IL-22, IFNγ, CXCR4, CXCR5, GM-CSF, G-CSF and G-CSFR; detecting the interaction of one or more binding moieties that specifically bind to the one or more biomarkers, thereby detecting the abundance of the biomarkers in the subject sample; and obtaining a relative abundance of the one or more biomarkers in the subject sample by comparison to a baseline abundance of the biomarker; and administering a dose of a combination therapy comprising an anti-TNF treatment and an anti-IL-17 treatment when the abundance of one or more biomarkers is modulated.

In various embodiments, the dose of combination therapy is administered to the subject when the one or more biomarkers are gene products selected from the group consisting of LIF, IL-1RA, IL-10, IL-21 and CXCR5 and wherein the abundance of the biomarker in the sample is less than the baseline abundance. In various embodiments, the dose of combination therapy is administered to the subject when the one or more biomarkers are gene products selected from the group consisting of CXCL1, CXCL2, CCL2, CXCL5, CXCL9, CXCL10, CCL23, TNF, IL-6, IL-22, IFNγ, CXCR4, GM-CSF, G-CSF and G-CSFR and wherein the abundance of the biomarker in the sample is greater than the baseline abundance.

The description also provides a method of determining an increased risk of an inflammatory disorder in a subject, the method comprising the steps of determining a modulation of expression of one or more biomarkers in a sample from the subject, wherein the one or more biomarkers are gene products selected from the group consisting of LIF, CXCL1, CXCL2, CXCL5, CXCL9, CXCL10, CCL2, CCL23, IL-1Ra, TNF, IL-6, IL-10, IL-21, IL-22, IFNγ, CXCR4, CXCR5, GM-CSF, G-CSF and G-CSFR; detecting the interaction of one or more binding moieties that specifically bind to the one or more biomarkers, thereby detecting the relative abundance of the one or more biomarkers in the subject sample; and obtaining a relative abundance of the one or more biomarkers in the subject sample by comparison to a baseline abundance of the one or more biomarker; wherein the subject has an increased risk of an inflammatory disorder when the abundance of the one or more biomarkers is modulated.

In various embodiments, the subject has an increased risk of an inflammatory disorder when the one or more biomarkers are gene products selected from the group consisting of LIF, IL-1RA, IL-10, IL-21 and CXCR5 and wherein the abundance of the biomarker in the sample is less than the baseline abundance. In various embodiments, the subject has an increased risk of an inflammatory disorder when the one or more biomarkers are gene products selected from the group consisting of CXCL1, CXCL2, CCL2, CXCL5, CXCL9, CXCL10, CCL23, TNF, IL-6, IL-22, IFNγ, CXCR4, GM-CSF, G-CSF and G-CSFR and wherein the abundance of the biomarker in the sample is greater than the baseline abundance.

In other embodiments. the baseline abundance of the biomarker is the abundance of the biomarker in a healthy subject. In certain embodiments, the healthy subject is not experiencing the inflammatory disorder. In certain embodiments, the baseline abundance of the biomarker is the average abundance of the biomarker in two or more healthy subjects. In certain embodiments, the baseline abundance of the biomarker is the abundance of the biomarker in the treated subject before the subject experienced the inflammatory disorder. In certain embodiments, the baseline abundance of the biomarker is the abundance of the biomarker in the treated subject before the subject was experiencing symptoms of the inflammatory disorder.

In other embodiments, further including normalizing the abundance of the biomarker using one or more control biomarkers, wherein the one or more control biomarkers are gene products selected from the group consisting of GM-CSFR, CXCL4, CXCL8, IL-1β, IL-17A, IL-17F and CXCR1.

In other embodiments, the subject is a human subject.

### BRIEF DESCRIPTION OF THE DRAWINGS

FIG. 1, panel A is a protocol for a mouse collagen induced arthritis (CIA) model involving injecting collagen II and complete Freund's adjuvant (CFA) into subjects at day zero. For one group, subjects were either administered a prophylactic dosing of anti-TNF antibody, anti-IL-17 antibody or anti-TNF/anti-IL-17 DVD-Ig protein (at day 20 after collagen II/CFA injection) one day prior to injection of one milligram of zymosan (at day 21 after collagen II/CFA injection). For another group, a therapeutic dose of anti-TNF antibody, anti-IL-17 antibody or anti-TNF/anti-IL-17 DVD-Ig protein was administered to subjects (at days 21-24 after collagen II/CFA injection) three to seven days after an injection of zymosan (at day 21 after collagen II/CFA injection). Paw swelling (millimeter cubed divided by mean arthritis score; mm³/MAS) was analyzed using calipers over a period of days.

FIG. 1, panel B is a graph showing mean arthritic score (ordinate) as a function of time (abscissa) of subjects in a CIA model administered a prophylatic dose of antibodies. The murine subjects were administered either: 8C11 anti-TNF antibody; MAB421 anti-IL-17 antibody; or a mixture of both 8C11 anti-TNF antibody and MAB421 anti-IL-17 antibody. Control subjects were administered vehicle only.

FIG. 1, panel C is a graph showing mean arthritic score (ordinate; millimeter cubed; mm³) as a function of time (abscissa) of subjects in a CIA model administered a therapeutic dose of antibodies. The murine subjects were administered either: 8C11 anti-TNF antibody; MAB421 anti-IL-17 antibody; or a mixture of both 8C11 anti-TNF antibody and MAB421 anti-IL-17 antibody. Control subjects were administered vehicle only.

FIG. 1, panel D is a graph showing micro CT analyzed bone volume (mm³; ordinate) of tarsal bone of subjects in a CIA model administered a dose of antibodies. The subjects were administered either: 8C11 anti-TNF antibody; MAB421 anti-IL-17 antibody; or a mixture of both 8C11 anti-TNF antibody and MAB421 anti-IL-17 antibody. Control subjects were administered vehicle only. Naive subjects were not administered any dose.

FIG. 1, panel E is a graph showing histological scores (ordinate) of rear paws of subjects in a CIA model administered a dose of antibodies. The subjects were administered either: 8C11 anti-TNF antibody; MAB421 anti-IL-17 antibody; or a mixture of both 8C11 anti-TNF antibody and MAB421 anti-IL-17 antibody. Control subjects were administered vehicle only.

FIG. 2 is a schematic representation of anti-murine TNF/IL-17 Dual Variable Domain Immunoglobulin (DVD-Ig) binding protein composed of 8C11 mouse anti-TNF antibody and 10F7M11 mouse anti-IL-17 antibody.

FIG. 3 is a schematic outlining the study design for using anti-TNF/IL-17 DVD-Ig binding protein in a murine CIA model. Subjects were immunized with collagen and received a zymosan boost to promote the onset of the disease. Twenty four days after the collagen immunization, the subjects were administered DVD-Ig protein twice a week for three weeks (i.e., 21 days). Seven days after the first treatment administration of antibodies (i.e., either 8C11 anti-TNF antibody only, MAB421 anti-IL-17 antibody only, 8C11/10F7M11 anti-TNF/IL-17 DVD DVD-Ig protein only, or a mixture of the anti-TNF antibody and the anti-IL-17 antibody) the molecular response of the treatment was analyzed using homogenates of the paw of the subjects. Twenty one days after the first treatment with antibodies, animals were analyzed for swelling and bone histology to determine the efficacy of the specific treatments.

FIG. 4, panel A is a graph showing change in paw thickness (ordinate; change in millimeters) as a function of time in animals administered 8C11/10F7M11 anti-TNF/IL-17 DVD-Ig protein (abscissa). Control animals were administered vehicle only.

FIG. 4, panel B is a graph showing AUC of change in paw thickness (ordinate; millimeters) in animals administered 8C11/10F7M11 anti-TNF/IL-17 DVD-Ig protein (abscissa). Control animals were administered vehicle only.

FIG. 5 is a graph showing histology score (ordinate) for inflammation, cartilage, and bone in subjects administered 8C11/10F7M11 anti-TNF/IL-17 DVD-Ig protein. Control subjects were administered vehicle only.

FIG. 6 a graph showing bone volume (ordinate; millimeters cubed, mm³) in animals administered 8C11/10F7M11 anti-TNF/IL-17 DVD-Ig protein. Control animals were administered vehicle only. Naïve subjects were not administered any DVD-Ig protein or vehicle.

FIG. 7, panel A is a graph showing amount of CXCL-10 in tissue (ordinate; picograms per gram of tissue) of animals administered either: 8C11 anti-TNF antibody, 10F7M11 anti-IL-17 antibody, 8C11/10F7M11 anti-TNF/IL-17 DVD-Ig protein, or a mixture of the 8C11 anti-TNF antibody and the 10F7M11 anti-IL-17 antibody. Control animals were administered vehicle only. Naïve subjects were not administered any DVD-Ig protein or vehicle.

FIG. 7, panel B is a graph showing percent of amount of CXCL-10 in tissue of animals administered vehicle (ordinate; picograms per gram of tissue) compared to subjects administered different concentrations (abscissa; 0.1, 1 or 10 mg/kg) 8C11/10F7M11 anti-TNF/IL-17 DVD-Ig protein.

FIG. 8, panel A is a graph showing CXCL-1 in tissue (ordinate; picograms per gram of tissue) of animals administered either: 8C11 anti-TNF antibody, 10F7M11 anti-IL-17 antibody, 8C11/10F7M11 anti-TNF/IL-17 DVD-Ig protein, or a mixture of 8C11 anti-TNF antibody and 10F7M11 anti-IL-17 antibody. Control animals were administered vehicle only. Naïve subjects were not administered any DVD-Ig protein or vehicle.

FIG. 8, panel B is a graph showing amount of CXCL-1 in paw homogenates of animals administered different dosages (abscissa; 0.1, 1 or 10 mg/kg mg/kg) of 8C11/10F7M11 anti-TNF/IL-17 DVD-Ig protein as a relative percentage of amount of CXCL-1 in homogenates of control subjects administered only vehicle. Note that the percent amount of CXCL-1 in naïve subjects was set to 0%.

FIG. 8, panel C is a graph showing granulocyte colony-stimulating factor (G-CSF) in tissue (ordinate; picograms per gram of tissue) of animals administered either: 8C11 anti-TNF antibody, 10F7M11 anti-IL-17 antibody, 8C11/10F7M11 anti-TNF/IL-17 DVD-Ig protein, or a mixture of the anti-TNF antibody and anti-IL-17 antibody. Control subjects were administered vehicle only. Naïve subjects were not administered any DVD-Ig protein or vehicle.

FIG. 8, panel D is a graph showing amount of G-CSF in paw homogenates of animals administered different dosages (abscissa; 0.1, 1 or 10 mg/kg mg/kg) of 8C11/10F7M11 anti-TNF/IL-17 DVD-Ig protein as a relative percentage of amount of G-CSF in homogenates of control subjects administered only vehicle. Note that the percent amount of G-CSF in naïve subjects was set to 0%.

FIG. 9, panels A-C are graphs showing that following a single dose of ABT-122, CXCR4 expression was significantly decreased on T cells, B cells, and monocytes in healthy volunteers.

FIG. 9, panel A is a graph showing percent change in T cell CXCR4 expression (ordinate) for PBMCs from healthy subjects administered a single dose (1.5 mg/kg) of ABT-122 days earlier (abscissa).

FIG. 9, panel B is a graph showing percent change in B cell CXCR4 expression (ordinate) for PBMCs from healthy subjects administered a single dose (1.5 mg/kg) of ABT-122 days earlier (abscissa).

FIG. 9, panel C is a graph showing percent change in monocyte cell CXCR4 expression (ordinate) for PBMCs from healthy subjects administered a single dose (1.5 mg/kg) of ABT-122 days earlier (abscissa).

FIG. 10 is a graph showing percentage change in T cell CXCR5 expression for PBMCs from healthy subjects administered a single dose (1.5 mg/kg) of ABT-122 days earlier (abscissa). CXCR5 expression was significantly increased on T cells in healthy volunteers following a single dose of ABT-122.

FIG. 11 is a graph showing percentage change in T cell CXCR1 expression for PBMCs from healthy subjects administered a single dose (1.5 mg/kg) of ABT-122 days earlier (abscissa).

FIG. 12 is a graph showing GM-CSF CellTiter-Glo ratio (calculated concentration of GM-CSF divided by the relative luminescent units for each sample; ordinate) for PBMCs from healthy subjects administered a single dose (1.5 mg/kg) of ABT-122 days earlier (abscissa). GM-CSF levels were significantly decreased following ex vivo LPS stimulation after ABT-122 administration to healthy volunteers.

FIG. 13 is a graph showing IL-10 CellTiter-Glo ratio (ordinate) for PBMCs from healthy subjects administered a single dose (1.5 mg/kg) of ABT-122 days earlier (abscissa). Ex vivo LPS-stimulated IL-10 levels were significantly increased following ABT-122 administration in healthy volunteers.

FIG. 14, is a graph showing IL-1RA CellTiter-Glo ratio (ordinate) for PBMCs from healthy subjects administered a single dose (1.5 mg/kg) of ABT-122 days earlier (abscissa). Ex vivo LPS-stimulated IL-1RA levels were significantly increased following ABT-122 administration in healthy volunteers.

FIG. 15 is a graph showing TNF CellTiter-Glo ratio (ordinate) for PBMCs from healthy subjects administered a single dose (1.5 mg/kg) of ABT-122 days earlier (abscissa). Ex vivo LPS-stimulated TNF levels were significantly decreased following ABT-122 administration in healthy volunteers.

FIG. 16 is a graph showing IFNγ CellTiter-Glo ratio (ordinate) for PBMCs from healthy subjects administered a single dose (1.5 mg/kg) of ABT-122 days earlier (abscissa). Ex vivo anti-CD3 plus anti-CD28-stimulated IFNγ levels were significantly decreased following ABT-122 administration in healthy volunteers.

FIG. 17 is a graph showing IL-22 CellTiter-Glo ratio (ordinate) for PBMCs from healthy subjects administered a single dose (1.5 mg/kg) of ABT-122 days earlier (abscissa). Ex vivo anti-CD3 plus anti-CD28-stimulated IL-22 levels were significantly decreased following ABT-122 administration in healthy volunteers

FIG. 18 is a graph showing GM-CSF CellTiter-Glo ratio (ordinate) for PBMCs from healthy subjects administered a single dose (1.5 mg/kg) of ABT-122 days earlier (abscissa). Ex vivo anti-CD3 plus anti-CD28-stimulated GM-CSF levels were significantly decreased following ABT-122 administration in healthy volunteers

FIG. 19 is a graph showing LIF CellTiter-Glo ratio (ordinate) for PBMCs from healthy subjects administered a single dose (1.5 mg/kg) of ABT-122 days earlier (abscissa). Ex vivo anti-CD3 plus anti-CD28-stimulated LIF levels were significantly increased following ABT-122 administration in healthy volunteers.

FIG. 20 is a graph showing IL-21 CellTiter-Glo ratio (ordinate) for PBMCs from healthy subjects administered a single dose (1.5 mg/kg) of ABT-122 days earlier (abscissa). Ex vivo anti-CD3 plus anti-CD28-stimulated IL-21 levels were significantly increased following ABT-122 administration in healthy volunteers.

FIG. 21 is a graph showing IL-1RA CellTiter-Glo ratio (ordinate) for PBMCs from healthy subjects administered a single dose (1.5 mg/kg) of ABT-122 days earlier (abscissa). Ex vivo anti-CD3 plus anti-CD28-stimulated IL-1RA levels were significantly increased following ABT-122 administration in healthy volunteers.

FIG. 22, panel A is a graph showing CXCL1 CellTiter-Glo ratio for PBMCs from healthy subjects administered a single dose (1.5 mg/kg) of ABT-122 days earlier (abscissa).

FIG. 22, panel B is a graph showing G-CSF CellTiter-Glo ratio for PBMCs from healthy subjects administered a single dose (1.5 mg/kg) of ABT-122 days earlier (abscissa).

FIG. 23 is a graph showing fold changes in serum CXCL9 levels relative to baseline (day 1) in stable RA subjects administered 8 weekly doses (1.5 mg/kg or 3.0 mg/kg) of ABT-122. CXCL9 serum levels were significantly decreased from 3-64 days following initiation of ABT-122 administration in stable RA subjects (*p<0.05).

FIG. 24 is a graph showing fold changes in serum CXCL10 levels relative to baseline (day 1) in stable RA subjects administered 8 weekly doses (1.5 mg/kg or 3.0 mg/kg) of ABT-122. CXCL10 serum levels were significantly decreased from 3-64 days following initiation of ABT-122 administration in stable RA subjects (*p<0.05).

FIG. 25 is a graph showing fold changes in serum CCL23 levels relative to baseline (day 1) in stable RA subjects administered 8 weekly doses (1.5 mg/kg or 3.0 mg/kg) of ABT-122. CCL23 serum levels were significantly decreased from 78-92 days following initiation of ABT-122 administration at the 3.0 mg/kg dose in stable RA subjects (*p<0.05).

FIG. 26 is a graph showing fold changes in serum soluble e-selectin levels relative to baseline (day 1) in stable RA subjects administered 8 weekly doses (1.5 mg/kg or 3.0 mg/kg) of ABT-122. Soluble e-selectin serum levels were significantly decreased from 15-92 days following initiation of ABT-122 administration at the 3.0 mg/kg dose in stable RA subjects (*p<0.05).

FIG. 27, panels A-C are graphs showing geometric mean of G-CFSR (ordinate) on B cells, monocytes, and T cells from healthy subjects, who days earlier (abscissa) were intravenously administered a single dose (3 mg/kg) of ABBV-257, a TNF/IL-17 DVD-Ig binding protein, or placebo.

FIG. 28, panels A-C are graphs showing geometric mean of GM-CFSR (ordinate) on B cells, monocytes, and T cells from healthy subjects, who days earlier (abscissa) were intravenously administered a single dose (3 mg/kg) of ABBV-257, a TNF/IL-17 DVD-Ig binding protein, or placebo.

FIG. 29, panels A-C are graphs showing geometric mean of CXCR4 (ordinate) on B cells, monocytes, and T cells from healthy subjects, who days earlier (abscissa) were intravenously administered a single dose (3 mg/kg) of ABBV-257 binding protein or a placebo.

FIG. 30, panels A-C are graphs showing geometric mean of CXCR5 (ordinate) on B cells, monocytes, and T cells from healthy subjects, who days earlier (abscissa) were intravenously administered a single dose (3 mg/kg) of ABBV-257 binding protein or a placebo.

FIG. 31, panels A-C are graphs showing geometric mean of G-CFSR (ordinate) on B cells, monocytes, and T cells from healthy subjects, who days earlier (abscissa) were subcutaneously administered a single dose (3 mg/kg) of ABBV-257 binding protein or a placebo.

FIG. 32, panels A-C are graphs showing geometric mean of GM-CFSR (ordinate) on B cells, monocytes, and T cells from healthy subjects, who days earlier (abscissa) were subcutaneously administered a single dose (3 mg/kg) of ABBV-257 binding protein or a placebo.

FIG. 33, panels A-C are graphs showing geometric mean of CXCR4 (ordinate) on B cells, monocytes, and T cells from healthy subjects, who days earlier (abscissa) were subcutaneously administered a single dose (3 mg/kg) of ABBV-257 binding protein or a placebo.

FIG. 34, panels A-C are graphs showing geometric mean of CXCR5 (ordinate) on B cells, monocytes, and T cells from healthy subjects, who days earlier (abscissa) were subcutaneously administered a single dose (3 mg/kg) of ABBV-257 binding protein or a placebo.

FIG. 35, panels A and B are graphs showing showing IL-1Ra concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein or a placebo. Data show ex vivo cytokine responses.

FIG. 36, panel A and B, are graphs showing GM-CSF concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein or a placebo. Data show ex vivo cytokine responses.

FIG. 37, panel A and B, are graphs showing IL-21 concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein or a placebo. Data show ex vivo cytokine responses.

FIG. 38, panels A and B, are graphs showing IL-10 concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein, or a placebo. Data show ex vivo cytokine responses.

FIG. 39, panels A and B, are graphs showing LIF concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein or a placebo. Data show ex vivo cytokine responses.

FIG. 40, panels A and B, are graphs showing IFNγ (IFN gamma; IFNg) concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein or a placebo. Data show ex vivo cytokine responses.

FIG. 41, panels A and B, are graphs showing TNF concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein, or a placebo. Data show ex vivo cytokine responses.

FIG. 42, panels A and B, are graphs showing IL-17F concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein, or a placebo. Data show ex vivo cytokine responses.

FIG. 43, panels A and B, are graphs showing G-CSF concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein or a placebo. Data show ex vivo cytokine responses.

FIG. 44, panels A and B, are graphs showing IL-17A concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein or a placebo. Data show ex vivo cytokine responses.

FIG. 45, panels A and B, are graphs showing IL-1β concentration normalized by Cell Titer Glo value (ordinate) for collected, stimulated (with LPS or CD3/CD28) and analyzed PBMCs collected from healthy subjects, who days earlier (abscissa) were subcutaneously administered a single dose (3 mg/kg) of ABBV-257 binding protein or a placebo. Data show ex vivo cytokine responses.

FIG. 46, panels A and B, are graphs showing IL-1Ra concentration normalized by Cell Titer Glo value (ordinate) for collected, stimulated (with LPS or CD3/CD28) and analyzed PBMCs collected from healthy subjects, who days earlier (abscissa) were intravenously administered a single dose (3 mg/kg) of ABBV-257 binding protein, or a placebo. Data show ex vivo cytokine responses.

FIG. 47, panels A and B, are graphs showing GM-CSF concentration normalized by Cell Titer Glo value (ordinate) for collected, stimulated (with LPS or CD3/CD28) and analyzed PBMCs collected from healthy subjects, who days earlier (abscissa) were intravenously administered a single dose (3 mg/kg) of ABBV-257 binding protein or a placebo. Data show ex vivo cytokine responses.

FIG. 48, panels A and B, are graphs showing IL-21 concentration normalized by Cell Titer Glo value (ordinate) for collected, stimulated (with LPS or CD3/CD28) and analyzed PBMCs collected from healthy subjects, who days earlier (abscissa) were intravenously administered a single dose (3 mg/kg) of ABBV-257 binding protein or a placebo. Data show ex vivo cytokine responses.

FIG. 49, panels A and B, are graphs showing IL-10 concentration normalized by Cell Titer Glo value (ordinate) for collected, stimulated (with LPS or CD3/CD28) and analyzed PBMCs collected from healthy subjects, who days earlier (abscissa) were intravenously administered a single dose (3 mg/kg) of ABBV-257 binding protein or a placebo. Data show ex vivo cytokine responses.

FIG. 50, panels A and B, are graphs showing LIF concentration normalized by Cell Titer Glo value (ordinate) for collected, stimulated (with LPS or CD3/CD28) and analyzed PBMCs collected from healthy subjects, who days earlier (abscissa) were intravenously administered a single dose (3 mg/kg) of ABBV-257 binding protein, or a placebo. Data show ex vivo cytokine responses.

FIG. 51, panels A and B, are graphs showing IFNγ (IFN gamma; IFNg) concentration normalized by Cell Titer Glo value (ordinate) for collected, stimulated (with LPS or CD3/CD28) and analyzed PBMCs collected from healthy subjects, who days earlier (abscissa) were intravenously administered a single dose (3 mg/kg) of ABBV-257 binding protein, or a placebo. Data show ex vivo cytokine responses.

FIG. 52, panels A and B, are graphs showing TNF concentration normalized by Cell Titer Glo value (ordinate) for collected, stimulated (with LPS or CD3/CD28) and analyzed PBMCs collected from healthy subjects, who days earlier (abscissa) were intravenously administered a single dose (3 mg/kg) of ABBV-257 binding protein or a placebo. Data show ex vivo cytokine responses.

### DETAILED DESCRIPTION OF THE INVENTION

The present invention is based on the identification of novel biomarkers for anti-TNF and anti-IL-17 combination therapies. Specifically, the present invention is based, at least in part, on the observation that a combination therapy of an anti-TNF treatment and an anti-IL-17 treatment modulates (e.g., lowers or increases) the level of expression of LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid in a subject having an inflammatory disease, relative to a their expression in a control subject or control subject population, indicating that the combination therapy is, or will be, effective in treating the subject for the inflammatory disease. Accordingly, the present invention is useful for (i) determining whether a subject will respond to a combination therapy comprising an anti-TNF treatment and an anti-IL-17 treatment; (ii) monitoring the effectiveness of a combination therapy comprising an anti-TNF treatment and an anti-IL-17 treatment; (iii) selecting a subject for participation in a clinical trial for a combination therapy comprising an anti-TNF treatment and an anti-IL-17 treatment; (iv) identifying a combination therapy comprising an anti-TNF treatment and an anti-IL-17 treatment for treating a subject having an inflammatory disease and/or identifying candidate substances that could be used to treat inflammatory diseases.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms, e.g., those characterized by “a” or “an”, shall include pluralities, e.g., one or more markers (e.g., biomarkers); “some”, “certain”, and “various”. In this application, the use of “or” means “and/or”, unless stated or differentiated otherwise. Furthermore, the use of the terms “including” and “comprising,” as well as other forms of the terms, such as “includes”, “included”, “comprises”, and “comprised of”, are not limiting. Also, terms such as “element” or “component” encompass both elements and components comprising one unit and elements and components that comprise more than one unit unless specifically stated otherwise.

The phrase “determining whether a subject having an inflammatory disease will respond to treatment with a combination therapy comprising an anti-TNF treatment and an anti-IL-17 treatment” refers to assessing the likelihood that treatment of a subject with a dose of the combination therapy will be therapeutically effective (e.g., provide a therapeutic benefit to the subject) or will not be therapeutically effective in the subject. Assessment of the likelihood that treatment will or will not be therapeutically effective typically can be performed before treatment has begun or before treatment is resumed. Alternatively or in combination, assessment of the likelihood of effective treatment can be performed during treatment, e.g., to determine whether treatment should be continued or discontinued.

The term “anti-TNF treatment” includes any treatment for a TNF associated disease and/or any treatment that affects (e.g., inhibits) the TNF pathway. This term includes TNF antagonists that have the effect of binding to or neutralizing, inhibiting, reducing, or negatively modulating the activity of tumor necrosis factor (TNF). In an embodiment, the anti-TNF treatment comprises an anti-TNF binding protein. In an embodiment, the anti-TNF treatment can comprise an anti-TNF antibody, or an antigen binding fragment thereof. In an embodiment, an antibody is a murine antibody, a human antibody, a humanized antibody, a bispecific antibody, a chimeric antibody, a Fab, a Fab′, a F(ab′)₂, an ScFv, an SMIP, an affibody, an avimer, a versabody, a nanobody, a domain antibody, and an antigen binding fragment of any of the foregoing.

In an embodiment, the anti-TNF antibody comprises, e.g., a human anti-TNFα antibody, e.g., Adalimumab, or an antigen binding fragment thereof (see U.S. Pat. No. 6,090,382). In another embodiment, the anti-TNF antibody comprises a humanized anti-TNF antibody, e.g., infliximab, or an antigen binding fragment thereof. In another embodiment, the anti-TNF binding protein comprises a fusion protein, e.g., etanercept, or an antigen binding fragment thereof. In other embodiments, the anti-TNF treatment comprises methotrexate, an analog thereof, or a pharmaceutically acceptable salt thereof. In an embodiment, the anti-TNF comprises a multispecific binding protein. In an embodiment, the multispecific binding protein comprises a dual variable domain (DVD) binding protein such as, for example, a dual variable domain immunoglobulin (DVD-Ig) molecule, a half-body DVD-Ig (hDVD-Ig) molecule, a triple variable domain immunoglobulin (tDVD-Ig) molecule, a receptor variable domain immunoglobulin (rDVD-Ig) molecule, a polyvalent DVD-Ig (pDVD-Ig) molecule, a monobody DVD-Ig (mDVD-Ig) molecule, a cross over (coDVD-Ig) molecule, a blood brain barrier (bbbDVD-Ig) molecule, a cleavable linker DVD-Ig (clDVD-Ig) molecule, or a redirected cytotoxicity DVD-Ig (rcDVD-Ig) molecule.

The term“anti-IL-17 treatment” includes any treatment for an IL-17 associated disease and/or any treatment that affects (e.g., inhibits) the IL-17 pathway. This term includes IL-17 antagonists that have the effect of binding to or neutralizing, inhibiting, reducing, or negatively modulating the activity of interleukin 17 (IL-17). In an embodiment, the anti-IL-17 treatment comprises an anti-IL-17 binding protein. In another example, the anti-IL-17 binding protein comprises a fusion protein. In an embodiment, the anti-ILIL-1717 treatment comprises an anti-IL-17 antibody, or an antigen binding fragment thereof. In an embodiment, the anti-IL-17 antibody comprises a human antibody, e.g., secukinumab and RG7624, or an antigen binding fragment thereof. In an embodiment, the anti-IL-17 antibody comprises a humanized antibody, for example ixekizumab, 10F7, B6-17, or an antigen binding fragment thereof. In other embodiments, the anti-IL-17 treatment comprises methotrexate, an analog thereof, or a pharmaceutically acceptable salt thereof. In an embodiment, the anti-IL-17 can include a multispecific binding protein, as described above and, in more detail, below.

Antibodies used in immunoassays to determine the level of expression of the biomarker, may be labeled with a detectable label. The term “labeled”, with regard to the probe or antibody, is intended to encompass direct labeling of the probe or antibody by incorporation of a label (e.g., a radioactive atom), coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In one embodiment, the antibody is labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair (e.g., biotin-streptavidin), or an antibody fragment (e.g., a single-chain antibody, or an isolated antibody hypervariable domain) which binds specifically with the biomarker is used.

The phrase “inflammatory disease” refers to a disease or disorder characterized by chronic or acute inflammation. Numerous inflammatory diseases are known in the art, such as arthritis, including rheumatoid arthritis, osteoarthritis, psoriatic arthritis, juvenile idiopathic arthritis; necrotizing enterocolitis (NEC); gastroenteritis; intestinal flu; stomach flu; pelvic inflammatory disease (PID); emphysema; pleurisy; pyelitis; pharyngitis; sore throat; angina; acne vulgaris; rubor; urinary tract infection; appendicitis; bursitis; colitis; cystitis; dermatitis; phlebitis; rhinitis; tendonitis; tonsillitis; vasculitis; asthma; autoimmune diseases; celiac disease; chronic prostatitis; glomerulonephritis; hypersensitivities; inflammatory bowel diseases; pelvic inflammatory disease; reperfusion injury; sarcoidosis; transplant rejection; vasculitis; interstitial cystitis; hay fever; periodontitis; atherosclerosis; psoriasis; ankylosing spondylitis; juvenile idiopathic arthritis; Behcet's disease; spondyloarthritis; uveitis; systemic lupus erythematosus, and some cancers (e.g., gallbladder carcinoma).

The terms “marker” or “biomarker” are used interchangeably herein to mean a substance that is used as an indicator of a biologic state, e.g., proteins, genes, DND, cDNA, messenger RNAs (mRNAs, microRNAs (miRNAs)); heterogeneous nuclear RNAs (hnRNAs), and proteins, or portions thereof.

The terms “level of expression” or “expression pattern” refers to a quantitative or qualitative summary of the expression of one or more markers or biomarkers in a subject, such as in comparison to a standard or a control.

The term “baseline abundance” as used herein means the level of biomarker present in a sample as a comparator to a subject or a sample that has been treated with an anti-TNF and anti-IL-17 treatment. In an embodiment, the baseline abundance refers to the level of biomarker in a normal individual or population of individuals. In an embodiment, the baseline abundance refers to the level of biomarkers in a subject with inflammation prior to treatment with the anti-TNF and anti-IL-17 treatment. In an embodiment, the baseline abundance refers to the level of biomarkers in a healthy tissue from a subject with inflammation. In an embodiment, the baseline abundance refers to the level of biomarkers in a healthy tissue from a subject that was collected from the subject during an period in which the subject was not experiencing symptoms of inflammation. Thus, regardless of the “baseline abundance” measurement chosen, the biomarkers of the invention that are increased in subject samples (e.g., serum or LPS stimulated subject PBMCs) following anti-TNF and anti-IL-17 treatment (i.e., LIF, IL-RA, IL-10, IL-21, CXCR5) and the biomarkers of the invention that are decreased in subjects following anti-TNF and anti-IL-17 treatment (CXCL1, CXCL2, CCL2, CXCL5, CXCL9, CXCL10, CCL23, TNF, IL-6, IL-22, IFNγ, CXCR4, GM-CSF, G-CSF and G-CSFR) can be used across disease indications to determine responsiveness to the anti-TNF and anti-IL-17 treatment.

A “higher level of expression” or an “increase in the level of expression” (e.g., of CXCR5) refers to an expression level in a test sample that is greater than the standard error of the assay employed to assess expression, and is at least 50% greater, or two, three, four, five, six, seven, eight, nine, or ten or more times the expression level in a control sample (e.g., a sample from a healthy subject not afflicted with inflammatory disease, e.g., RA, and/or a sample from a subject(s) having slow disease progression and/or, the average expression level of CXCL10, CXCL1 and/or G-CSF in several control samples).

A “lower level of expression” or a “decrease in the level of expression” (e.g., GM-CSF, GM-CSFR, G-CSFR and/or G-CSF) refers to an expression level in a test sample that is less than the standard error of the assay employed to assess expression, and at least 50% greater, or two, three, four, five, six, seven, eight, nine, or ten or more times less than the expression level (e.g., of GM-CSFR,) in a control sample (e.g., a sample from a subject with rapid disease progression and/or a sample from the subject prior to administration of a portion of a therapy for inflammatory disease, e.g., RA, and/or the average expression level of CXCL10, CXCL1 and/or G-CSF in several control samples).

Chemokines may be divided into subfamilies based on conserved amino acid sequence motifs. Most chemokine family members include at least four conserved cysteine residues that form two intramolecular disulfide bonds. The chemokine subfamilies can be defined by the position of the first two of these cysteine residues.

The alpha (α) subfamily is also known as the CXC chemokines because of the presence of one amino acid separating the first two cysteine residues. This group can be further subdivided based on the presence or absence of a glu-leu-arg (ELR) amino acid motif immediately preceding the first cysteine residue. There are currently at least five CXC-specific receptors, which are designated CXCR1 to CXCR5. See U.S. Pat. No. 8,329,178. Thus, the term “CXCR4” refers to a CXC-Chemokine receptor 4, and the term “CXCR5” refers to a CXC-Chemokine receptor 5.

The term “CXCL1” refers to chemokine (C-X-C motif) ligand 1, which is a small cytokine belonging to the CXC chemokine family that was previously called GRO1 oncogene, GROα, KC, Neutrophil-activating protein 3 (NAP-3) and melanoma growth stimulating activity alpha (MSGA-α). In humans, this protein is encoded by the CXCL1 gene. In other animals, this protein is encoded by orthologous genes. The nucleotide and amino acid sequences of CXCL1 are known in the art and can be found for example, in publically available databases such as the NCBI GenBank. The human CXCL1 protein can be found under NCBI Reference Sequence NM_{—}001511.3. The amino acid and nucleotide sequences of the human CXCL1 protein and cDNA are shown below.

(SEQ ID NO: 78)
MARAALSAAPSNPRLLRVALLLLLLVAAGRRAAGASVATELRCQCLQTLQ
GIHPKNIQSVNVKSPGPHCAQTEVIATLKNGRKACLNPASPIVKKIIEKM
LNSDKSN
(SEQ ID NO: 77)
CACAGAGCCCGGGCCGCAGGCACCTCCTCGCCAGCTCTTCCGCTCCTCTC
ACAGCCGCCAGACCCGCCTGCTGAGCCCCATGGCCCGCGCTGCTCTCTCC
GCCGCCCCCAGCAATCCCCGGCTCCTGCGAGTGGCACTGCTGCTCCTGCT
CCTGGTAGCCGCTGGCCGGCGCGCAGCAGGAGCGTCCGTGGCCACTGAAC
TGCGCTGCCAGTGCTTGCAGACCCTGCAGGGAATTCACCCCAAGAACATC
CAAAGTGTGAACGTGAAGTCCCCCGGACCCCACTGCGCCCAAACCGAAGT
CATAGCCACACTCAAGAATGGGCGGAAAGCTTGCCTCAATCCTGCATCCC
CCATAGTTAAGAAAATCATCGAAAAGATGCTGAACAGTGACAAATCCAAC
TGACCAGAAGGGAGGAGGAAGCTCACTGGTGGCTGTTCCTGAAGGAGGCC
CTGCCCTTATAGGAACAGAAGAGGAAAGAGAGACACAGCTGCAGAGGCCA
CCTGGATTGTGCCTAATGTGTTTGAGCATCGCTTAGGAGAAGTCTTCTAT
TTATTTATTTATTCATTAGTTTTGAAGATTCTATGTTAATATTTTAGGTG
TAAAATAATTAAGGGTATGATTAACTCTACCTGCACACTGTCCTATTATA
TTCATTCTTTTTGAAATGTCAACCCCAAGTTAGTTCAATCTGGATTCATA
TTTAATTTGAAGGTAGAATGTTTTCAAATGTTCTCCAGTCATTATGTTAA
TATTTCTGAGGAGCCTGCAACATGCCAGCCACTGTGATAGAGGCTGGCGG
ATCCAAGCAAATGGCCAATGAGATCATTGTGAAGGCAGGGGAATGTATGT
GCACATCTGTTTTGTAACTGTTTAGATGAATGTCAGTTGTTATTTATTGA
AATGATTTCACAGTGTGTGGTCAACATTTCTCATGTTGAAACTTTAAGAA
CTAAAATGTTCTAAATATCCCTTGGACATTTTATGTCTTTCTTGTAAGGC
ATACTGCCTTGTTTAATGGTAGTTTTACAGTGTTTCTGGCTTAGAACAAA
GGGGCTTAATTATTGATGTTTTCATAGAGAATATAAAAATAAAGCACTTA
TAGAAAAAACTCGTTTGATTTTTGGGGGGAAACAAGGGCTACCTTTACTG
GAAAATCTGGTGATTTATAAAAAAAAAAAAAAAA

The term “CXCL2” refers to small cytokine belonging to the CXC chemokine family that is also called macrophage inflammatory protein 2-alpha (MIP2-alpha), Growth-regulated protein beta (Gro-beta) and Gro oncogene-2 (Gro-2). This chemokine is secreted by monocytes and macrophages and is chemotactic for polymorphonuclear leukocytes and hematopoietic stem cells. Wolpe, S. D., Sherry, B., Juers, D., Davatelis, G., Yurt, R. W., Cerami, A. Identification and characterization of macrophage inflammatory protein 2. Proc. Nat. Acad. Sci. 86: 612-616, 1989.

The term “CXCL4” refers to chemokine (C-X-C motif) ligand 4, also known as platelet factor 4 (PF4). This chemokine is released from alpha-granules of activated platelets during platelet aggregation, and promotes blood coagulation by moderating the effects of heparin-like molecules. Due to these roles, it is predicted to play a role in wound repair and inflammation. Eisman R, Surrey S, Ramachandran B, Schwartz E, Poncz M (July 1990). “Structural and functional comparison of the genes for human platelet factor 4 and PF4alt”. Blood 76 (2): 336-44.

The term “CXCL5” refers to chemokine (C-X-C motif) ligand 5, also known as epithelial neutrophil-activating peptide-78 (CXCL5), a member of the CXC chemokine family, is involved in angiogenesis, tumor growth, and metastasis. See U.S. publication number 2013/0101600.

The term “CXCL8” refers to chemokine (C-X-C motif) ligand 8, (also known as Interleukin-8, IL-8, monocyte-5 derived neutrophil chemotactic factor, MDNCF, Neutrophil-Activating Protein 1, NAP-1, lymphocyte-derived neutrophil-activating factor, LYNAP, neutrophil-activating factor, NAF, granulocyte chemotactic protein 1, GCP-1, Emoctakin), is known as a potent chemotactic inflammation-mediating factor exerting its activity not only on neutrophils, but also on lymphocytes, monocytes, endothelial cells, and fibroblasts.

The term “CXCL9” refers to chemokine (C-X-C motif) ligand 9. Also referred to as monokine induced by IFN-gamma (MIG), is a cytokine that affects the growth, movement and/or activation state of cells that participate in immune and inflammatory response. For example, CXCL9 is chemotactic for activated for T-cells. Shown below is a human CXCL9 amino acid sequence.

(SEQ ID NO: 80)

MKKSGVLFLL GIILLVLIGV QGTPVVRKGR CSCISTNQGT
IHLQSLKDLK QFAPSPSCEK IEIIATLKNG VQTCLNPDSA
DVKELIKKWE KQVSQKKKQKNGKKHQKKKVLKVRKSQRSRQKKTT

The term “CXCL10” refers to C-X-C motif chemokine 10, which is an 8.7 kDa protein that in humans is encoded by the CXCL10 gene. It is a small cytokine belonging to the CXC chemokine family. In humans, this protein is encoded by the CXCL10 gene. In other animals, this protein is encoded by orthologous genes. The nucleotide and amino acid sequences of CXCL10 are known in the art and can be found for example, in publically available databases such as the NCBI GenBank. The human CXCL10 protein can be found under NCBI Reference Sequence NP_{—}001556. The amino acid and nucleotide sequences, respectively, of the human CXCL10 protein and cDNA are shown below.

(SEQ ID NO: 21)

MNQTAILICCLIFLTLSGIQGVPLSRTVRCTCISISNQPVNPRSLEKLEI
IPASQFCPRVEIIATMKKKGEKRCLNPESKAIKNLLKAVSKERSKRSP

(SEQ ID NO: 79)

CTTTGCAGATAAATATGGCACACTAGCCCCACGTTTTCTGAGACATTCCT
CAATTGCTTAGACATATTCTGAGCCTACAGCAGAGGAACCTCCAGTCTCA
GCACCATGAATCAAACTGCCATTCTGATTTGCTGCCTTATCTTTCTGACT
CTAAGTGGCATTCAAGGAGTACCTCTCTCTAGAACTGTACGCTGTACCTG
CATCAGCATTAGTAATCAACCTGTTAATCCAAGGTCTTTAGAAAAACTTG
AAATTATTCCTGCAAGCCAATTTTGTCCACGTGTTGAGATCATTGCTACA
ATGAAAAAGAAGGGTGAGAAGAGATGTCTGAATCCAGAATCGAAGGCCAT
CAAGAATTTACTGAAAGCAGTTAGCAAGGAAAGGTCTAAAAGATCTCCTT
AAAACCAGAGGGGAGCAAAATCGATGCAGTGCTTCCAAGGATGGACCACA
CAGAGGCTGCCTCTCCCATCACTTCCCTACATGGAGTATATGTCAAGCCA
TAATTGTTCTTAGTTTGCAGTTACACTAAAAGGTGACCAATGATGGTCAC
CAAATCAGCTGCTACTACTCCTGTAGGAAGGTTAATGTTCATCATCCTAA
GCTATTCAGTAATAACTCTACCCTGGCACTATAATGTAAGCTCTACTGAG
GTGCTATGTTCTTAGTGGATGTTCTGACCCTGCTTCAAATATTTCCCTCA
CCTTTCCCATCTTCCAAGGGTACTAAGGAATCTTTCTGCTTTGGGGTTTA
TCAGAATTCTCAGAATCTCAAATAACTAAAAGGTATGCAATCAAATCTGC
TTTTTAAAGAATGCTCTTTACTTCATGGACTTCCACTGCCATCCTCCCAA
GGGGCCCAAATTCTTTCAGTGGCTACCTACATACAATTCCAAACACATAC
AGGAAGGTAGAAATATCTGAAAATGTATGTGTAAGTATTCTTATTTAATG
AAAGACTGTACAAAGTAGAAGTCTTAGATGTATATATTTCCTATATTGTT
TTCAGTGTACATGGAATAACATGTAATTAAGTACTATGTATCAATGAGTA
ACAGGAAAATTTTAAAAATACAGATAGATATATGCTCTGCATGTTACATA
AGATAAATGTGCTGAATGGTTTTCAAAATAAAAATGAGGTACTCTCCTGG
AAATATTAAGAAAGACTATCTAAATGTTGAAAGATCAAAAGGTTAATAAA
GTAATTATAACTAAGAAAAAAAAAAAA

The term “CCL2”, (also called Macrophage Chemotactic Protein-1 or MCP-1) refers to chemokine that in many cases displays chemotactic activity for monocytes and basophils. CCL2 is particularly highly expressed during inflammation, and is a potent monocyte as well as a lymphocyte chemoattractant. CCL2 activates CCR2 on rolling monocytes, triggering integrin mediated arrest. CCL2 is also one of the strongest histamine inducing factors. See U.S. Patent Publication No. 20090239799. The monomer or homodimer of CXCL2 are often in equilibrium. Furthermore, CXCL2 binds chemokine receptors, e.g., CCR1, CCR2 and CCR4. An exemplary human CCL2 (UniProt P13500) is shown below:

(SEQ ID NO: 56)

MKVSAALLCL LLIAATFIPQ GLAQPDAINA PVTCCYNFTN
RKISVQRLAS YRRITSSKCP KEAVIFKTIV AKEICADPKQ
KWVQDSMDHL DKQTQTPKT

The term “CCL23” or “chemokine (C-C motif) ligand 23” (also called CK-8, macrophage inflammatory protein 3, myeloid progenitor inhibitory factor 1, and C6 beta-chemokine) refers to a chemokine in the CC chemokine family. CCL23 may inhibit proliferation of myeloid progenitor cells in colony formation assays, has in certain circumstances to bind heparin and/or CCR1.

Cytokines, such as interleukin-1 (IL-1) and tumor necrosis factor (TNF), are molecules produced by a variety of cells, such as monocytes and macrophages, and are mediators of inflammatory processes. Interleukin-1 is a cytokine with a wide range of biological and physiological effects, including fever, prostaglandin synthesis (in, e.g., fibroblasts, muscle cells and endothelial cells), T-lymphocyte activation, and interleukin-2 production. The original members of the IL-1 superfamily are IL-1α, IL-1β, and the IL-1 Receptor antagonist (IL-1Ra, IL-1RA, IL-1ra, IL-1Rα).

The term “IL-1β” means a pro-inflammatory cytokines involved in immune defense against infection. IL-1β is produced by macrophages, monocytes and dendritic cells. See U.S. Pat. No. 8,841,417. A human mature IL-1β sequence is shown below:

(SEQ ID NO: 74)

APVRSLNCTLRDSQQKSLVMSGPYELKALHLQGQDMEQQVVESMSFVQGE
ESNDKIPVALGLKEKNLYLSCVLKDDKPTLQLESVDPKNYPKKKMEKRFV
FNKIEINNKLEFESAQFPNWYISTSQAENMPVFLGGTKGGQDITDFTMQF
VSS

Interleukin-1 receptor antagonist (IL-1ra) is a human protein that acts as an inhibitor of interleukin-1 activity. Certain IL-1ra receptor antagonists, including IL-1ra and variants and derivatives thereof, as well as methods of making and using them, are described, e.g., in U.S. Pat. Nos. 5,075,222; 6,599,873; 5,863,769; 5,858,355; 5,739,282; U.S. Pat. Nos. 5,922,573; 6,054,559; WO 91/08285; WO 91/17184; WO 91/17249; AU 9173636; WO 92/16221; WO 93/21946; WO 94/06457; WO 94/21275; FR 2706772; WO 94/21235; DE 4219626, WO 94/20517; WO 96/22793; WO 96/12022; WO 97/28828; WO 99/36541; WO 99/51744. The sequence for the human protein is shown below

(SEQ ID NO: 73)

RPSGRKSSKMQAFRIWDVNQKTFYLRNNQLVAGYLQGPNVNLEEKIDVVP
IEPHALFLGIHGGKMCLSCVKSGDETRLQLEAVNITDLSENRKQDKRFAF
IRSDSGPTTSFESAACPGWFLCTAMEADQPVSLTNMPDEGVMVTKFYFQE
DE

The terms “tumor necrosis factor” and “TNF” mean a naturally occurring cytokine that is involved in normal inflammatory and immune responses. Elevated levels of TNF play an important role in pathologic inflammation. Adalimumab binds specifically to TNF and neutralizes the biological function of TNF by blocking its interaction with the p55 and p75 cell surface TNF receptors. Adalimumab also modulates biological responses that are induced or regulated by TNF. After treatment with adalimumab, levels of acute phase reactants of inflammation (C-reactive protein [CRP] and erythrocyte sedimentation rate [ESR]) and serum cytokines rapidly decrease.

The term “IL-6” (also known as interferon-β2; B-cell differentiation factor; B-cell stimulatory factor-2; hepatocyte stimulatory factor; hybridoma growth factor; and plasmacytoma growth factor) means a multifunctional cytokine involved in numerous biological processes such as the regulation of the acute inflammatory response, the modulation of specific immune responses including B- and T-cell differentiation, bone metabolism, thrombopoiesis, epidermal proliferation, menses, neuronal cell differentiation, neuroprotection, aging, cancer, and the inflammatory reaction occurring in Alzheimer's disease. See Papassotiropoulos et al. (2001) Neurobiol. of Aging 22:863-871 and U.S. Patent Publication No. 20110293622.

The term “CXCL8” (also known as monocyte-derived neutrophil chemotactic factor, MDNCF, or neutrophil attractant/activation protein-1, NAP-1, IL-8), is a chemokine and a member of the cytokine family that displays chemotactic activity for specific types of leukocytes. CXCL8 is a member of the CXC chemokine family in which an amino acid is present between the first two of four highly conserved cysteine residues. CXCL8 is a polypeptide of which two predominant forms consist of 72 amino acids and 77 amino acids. See U.S. Patent Publication No. 20140170156.

The term “IL-10” (also known as interleukin-10 and human cytokine synthesis inhibitory factor) as used herein means an 18-kilodalton cytokine produced by subsets of T- and B-cells, i.e., macrophages and monocytes. See Roncarolo et al. (2004) J. Autoimmun 20(4): 269-72.

The term “IL-21” (also known as interleukin-21) means a type I cytokine that exerts pleiotropic effects on both innate and adaptive immune responses. IL-21 is produced by activated CD4+T cells, follicular T cells and Natural killer cells. See U.S. Patent Publication No. 20140170153. IL-21 is a potent modulator of cytotoxic T cells and NK cells. (Parrish-Novak et al. (2000) Nature 408:57-63; Parrish-Novak et al. (2002) J. Leuk. Biol. 72:856-863; Collins et al. (2003) Immunol. Res. 28:131-140; Brady et al. (2004) J. Immunol. 172:2048-58). T cell responses include enhancement of primary antigen response as modulation of memory T cell functions. Human mature IL-21 is a 133 amino acid polypeptide as provided below.

(SEQ ID NO: 75)

MQDRHMIR MRQLIDIVDQLK NYVNDLVPEF LPAPEDVETN
CEWSAFSCFQ KAQLKSANTG NNERIINVSI KKLKRKPPST
NAGRRQKHRL TCPSCDSYEK KPPKEFLERF KSLLQKMIHQ
HLSSRTHGSE DS

The term “IL-22” refers to interleukin-22, an α-helical cytokine. IL-22 binds to a heterodimeric cell surface receptor composed of IL-10R2 and IL-22R1 subunits. IL-22R is expressed on tissue cells, and it is absent on immune cells. Wolk K, Kunz S, Witte E, Friedrich M, Asadullah K, Sabat R (2004). “IL-22 increases the innate immunity of tissues”. Immunity 21 (2): 241-54. doi:10.1016/j.immuni.2004.

Granulocyte macrophage-colony stimulating factor (“GM-CSF”), a soluble secreted glycoprotein, is a potent immunomodulatory cytokine known to facilitate development and prolongation of both humoral and cellular mediated immunity. See U.S. Pat. Nos. 7,381,801 and 8,609,101. The term “GM-CSF” refers to granulocyte macrophage colony stimulation factor from any species or source and includes the full-length protein as well as fragments or portions of the protein. For example, a human GM-CSF is found in Genbank accession number BC108724.

The term granulocyte macrophage-colony stimulating factor receptor (“GM-CSFR”) means a receptor that binds GM-CSF that is a member of the cytokine receptor superfamily. GM-CSFR contains a cytokine receptor-homologous domain responsible for G-CSF binding, an immunoglobulin-like domain, three fibronectin type III domains, a transmembrane region, and an intracellular domain. See U.S. Pat. No. 6,716,811.

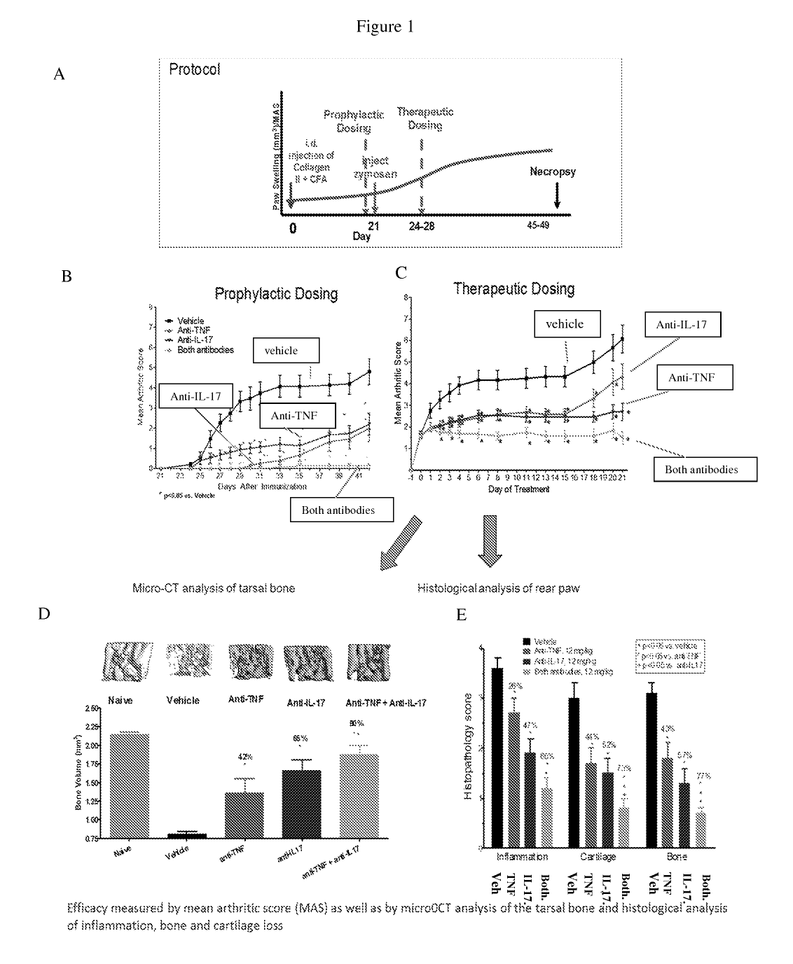

The term “G-CSF” means granulocyte colony-stimulating factor, which is a glycoprotein that induces the proliferation and differentiation of hematopoietic stem cells, promotes an increase in neutrophilic granulocytes in blood, and also stimulates the release of mature neutrophilic granulocytes from marrow, and activate neutrophilic granulocytes. G-CSF is a long polypeptide chain glycoprotein derived from monocytes and fibroblasts. The main spatial structure of G-CSF is helix with 103 out of 174 residues forming 4.alpha.-helixes, as shown in FIG. 1 (Hill et al. (1993) Proc. Natl. Acad. Sci. USA 90:5167-5171). See U.S. Pat. Nos. 8,785,597 and 8,716,239. Functionally, it is a cytokine and hormone, a type of colony-stimulating factor, and is produced by a number of different tissues. In humans, this protein is encoded by the GCSF gene. In other animals, this protein is encoded by orthologous genes. The nucleotide and amino acid sequences of G-CSF are known in the art and can be found for example, in publically available databases such as the NCBI GenBank. The human G-CSF protein can be found under NCBI Reference Sequence NP_{—}000750. The amino acid and nucleotide sequences of the human G-CSF protein and cDNA are shown below.

(SEQ ID NO: 25)

MAGPATQSPMKLMALQLLLWHSALWTVQEATPLGPASSLPQSFLLKCLEQ
VRKIQGDGAALQEKLVSECATYKLCHPEELVLLGHSLGIPWAPLSSCPSQ
ALQLAGCLSQLHSGLFLYQGLLQALEGISPELGPTLDTLQLDVADFATTI
WQQMEELGMAPALQPTQGAMPAFASAFQRRAGGVLVASHLQSFLEVSYRV
LRHLAQP

(SEQ ID NO: 76)

AGTCGTGGCCCCAGGTAATTTCCTCCCAGGCCTCCATGGGGTTATGTATA
AAGGCCCCCCTAGAGCTGGGCCCCAAAACAGCCCGGAGCCTGCAGCCCAG
CCCCACCCAGACCCATGGCTGGACCTGCCACCCAGAGCCCCATGAAGCTG
ATGGCCCTGCAGCTGCTGCTGTGGCACAGTGCACTCTGGACAGTGCAGGA
AGCCACCCCCCTGGGCCCTGCCAGCTCCCTGCCCCAGAGCTTCCTGCTCA
AGTGCTTAGAGCAAGTGAGGAAGATCCAGGGCGATGGCGCAGCGCTCCAG
GAGAAGCTGGTGAGTGAGTGTGCCACCTACAAGCTGTGCCACCCCGAGGA
GCTGGTGCTGCTCGGACACTCTCTGGGCATCCCCTGGGCTCCCCTGAGCA
GCTGCCCCAGCCAGGCCCTGCAGCTGGCAGGCTGCTTGAGCCAACTCCAT
AGCGGCCTTTTCCTCTACCAGGGGCTCCTGCAGGCCCTGGAAGGGATCTC
CCCCGAGTTGGGTCCCACCTTGGACACACTGCAGCTGGACGTCGCCGACT
TTGCCACCACCATCTGGCAGCAGATGGAAGAACTGGGAATGGCCCCTGCC
CTGCAGCCCACCCAGGGTGCCATGCCGGCCTTCGCCTCTGCTTTCCAGCG
CCGGGCAGGAGGGGTCCTGGTTGCCTCCCATCTGCAGAGCTTCCTGGAGG
TGTCGTACCGCGTTCTACGCCACCTTGCCCAGCCCTGAGCCAAGCCCTCC
CCATCCCATGTATTTATCTCTATTTAATATTTATGTCTATTTAAGCCTCA
TATTTAAAGACAGGGAAGAGCAGAACGGAGCCCCAGGCCTCTGTGTCCTT
CCCTGCATTTCTGAGTTTCATTCTCCTGCCTGTAGCAGTGAGAAAAAGCT
CCTGTCCTCCCATCCCCTGGACTGGGAGGTAGATAGGTAAATACCAAGTA
TTTATTACTATGACTGCTCCCCAGCCCTGGCTCTGCAATGGGCACTGGGA
TGAGCCGCTGTGAGCCCCTGGTCCTGAGGGTCCCCACCTGGGACCCTTGA
GAGTATCAGGTCTCCCACGTGGGAGACAAGAAATCCCTGTTTAATATTTA
AACAGCAGTGTTCCCCATCTGGGTCCTTGCACCCCTCACTCTGGCCTCAG
CCGACTGCACAGCGGCCCCTGCATCCCCTTGGCTGTGAGGCCCCTGGACA
AGCAGAGGTGGCCAGAGCTGGGAGGCATGGCCCTGGGGTCCCACGAATTT
GCTGGGGAATCTCGTTTTTCTTCTTAAGACTTTTGGGACATGGTTTGACT
CCCGAACATCACCGACGCGTCTCCTGTTTTTCTGGGTGGCCTCGGGACAC
CTGCCCTGCCCCCACGAGGGTCAGGACTGTGACTCTTTTTAGGGCCAGGC
AGGTGCCTGGACATTTGCCTTGCTGGACGGGGACTGGGGATGTGGGAGGG
AGCAGACAGGAGGAATCATGTCAGGCCTGTGTGTGAAAGGAAGCTCCACT
GTCACCCTCCACCTCTTCACCCCCCACTCACCAGTGTCCCCTCCACTGTC
ACATTGTAACTGAACTTCAGGATAATAAAGTGTTTGCCTCCAAAAAAAAA
AA

The term “G-CSFB” means granulocyte colony-stimulating factor receptor for granulocyte colony stimulating factor and a cytokine that plays a part in controlling the production, differentiation, and function of granulocytes. The encoded protein, which is a member of the family of cytokine receptors, may also function in some cell surface adhesion or recognition processes. Alternative names or synonyms of G-CSFR are CD114, CD114 antigen, colony stimulating factor 3 receptor (granulocyte), CSF3R, G-CSF receptor, G-CSF-R, GCSFR, and granulocyte colony-stimulating factor receptor.

Interferons (IFNs) play a variety of diferent various biological roles in antiviral defense, including cell growth, cell immunity etc. Interferon types IFN-α, IFN-β, IFN-w, and IFN-r are type I interferons and bind the type I IFN receptor. The term “IFN-γ” means a type II interferon and binds the type II IFN receptor (Pfeffer et al. (1998) Cancer Res. 58:2489-2499). IFN-β receptors are found on most cell types, except mature erythrocytes (Farrar and Schreiber (1993) Annu. Rev. Immunol. 11:571-611). IFN-β regulates a variety of biological functions, such as antiviral responses, cell growth, immune response, and tumor suppression, and IFN-.gamma may mediate a variety of human diseases. See U.S. Patent Publication No. 20140004127.

The term “LIF” (also known as leukaemia inhibitory factor) means a lymphoid factor which promotes long-term maintenance of embryonic stem cells by suppressing spontaneous differentiation. LIF has a number of other activities including cholinergic neuron differentiation, control of stem cell pluripotency, bone and fat metabolism, mitogenesis of certain factor dependent cell lines and promotion of megakaryocyte production in vivo. See U.S. Patent Publication Nos. 20050265964 and 20030004098.

Reference to a gene encompasses naturally occurring or endogenous versions of the gene, including wild type, polymorphic or allelic variants or mutants (e.g., germline mutation, somatic mutation) of the gene, which can be found in a subject. In an embodiment, the sequence of the biomarker gene is at least about 80%, at least about 85%, at least about 90%, at least about 91%, at least about 92%, at least about 93%, at least about 94%, at least about 95%, at least about 96%, at least about 97%, at least about 98%, or at least about 99% identical to a biomarker described herein, e.g., LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof. Sequence identity can be determined, e.g., by comparing sequences using NCBI BLAST (e.g., Megablast with default parameters).

In an embodiment, the level of expression of the biomarker is determined relative to a control or second sample, such as the level of expression of the biomarker in normal tissue (e.g., a range determined from the levels of expression of the biomarker observed in normal tissue samples). In an embodiment, the level of expression of the biomarker is determined relative to a control sample, such as the level of expression of the biomarker in samples from other subjects suffering from inflammatory disease or free of the inflammatory disease. For example, the level of expression of the biomarker in samples from other subjects can be determined to define levels of expression that correlate with sensitivity to treatment with an anti-TNF treatment and/or an anti-IL-17 treatment, and the level of expression of the biomarker in the sample from the subject of interest is compared to these levels of expression.

The term “binding moiety” refers to substances that specifically bind to a given molecule. In certain embodiments, binding moieties used according to the methods disclosed herein specifically bind for example to LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof.

The term “known standard level” or “control level” refers to an accepted or predetermined expression level of the biomarker, for example LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof, which is used to compare the expression level of the biomarker in a sample derived from a subject. In one embodiment, the control expression level of the biomarker is the average expression level of the biomarker in samples derived from a population of subjects, e.g., the average expression level of the biomarker in a population of subjects with or without an inflammatory disease, such as RA. In another embodiment, the population comprises a group of subjects who have not responded to a combination therapy with an anti-TNF treatment and an anti-IL-17 treatment, or a group of subjects who express the respective biomarker at high or normal levels. In another embodiment, the control level constitutes a range of expression of the biomarker in normal tissue. In another embodiment, the control level constitutes a range of expression of the biomarker in cells or plasma from a variety of subjects having RA. In another embodiment, “control level” refers also to a pre-treatment level in a subject. For example, a subject may be administered a candidate substance. In this instance, the control could be a subject or population of subjects who have not received the candidate substance. In certain embodiments, the control subject or population would have the same disease state, or absence of disease state as the test subject or population.

As further information becomes available as a result of routine performance of the methods described herein, population-average values for “control” level of expression of the biomarkers of the present invention may be used. In other embodiments, the “control” level of expression of the biomarkers may be determined by determining the expression level of the respective biomarker in a subject sample obtained from a subject before the suspected onset of inflammatory disease in the subject, from archived subject samples, and the like.

Control levels of expression of biomarkers of the invention may be available from publicly available databases. In addition, Universal Reference Total RNA (Clontech Laboratories) and Universal Human Reference RNA (Stratagene) and the like can be used as controls. For example, qPCR can be used to determine the level of expression of a biomarker, and an increase in the number of cycles needed to detect expression of a biomarker in a sample from a subject, relative to the number of cycles needed for detection using such a control, is indicative of a low level of expression of the biomarker.

The terms “antagonist” and “inhibitor” mean a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of human TNFα and IL-17. Antagonists and inhibitors of human TNFα and IL-17 may include, but are not limited to, proteins, nucleic acids, carbohydrates, or any other molecules, which bind to human TNFα and IL-17.

The term “effective amount” means the amount of a therapy that is sufficient to reduce or ameliorate the severity and/or duration of a disorder or one or more symptoms thereof; prevent the advancement of a disorder; cause regression of a disorder; prevent the recurrence, development, onset, or progression of one pr more symptoms associated with a disorder; detect a disorder; or enhance or improve the prophylactic or therapeutic effect(s) of another therapy (e.g., prophylactic or therapeutic agent).

The terms “patient” and “subject” mean an animal, such as a mammal, including a primate (for example, a human, a monkey, and a chimpanzee), a non-primate (for example, a cow, a pig, a camel, a llama, a horse, a goat, a rabbit, a sheep, a hamster, a guinea pig, a cat, a dog, a rat, a mouse, a whale), a bird and a fish. In an embodiment, the patient or subject is a human, such as a human being treated or assessed for a disease, disorder or condition; a human at risk for a disease, disorder or condition; and/or a human having a disease, disorder or condition.

The term “sample” means a quantity of a substance. The term “biological sample” means a quantity of a substance from a living thing or formerly living thing. Such substances include, but are not limited to, blood, plasma, serum, urine, amniotic fluid, synovial fluid, endothelial cells, leukocytes, monocytes, other cells, organs, tissues, bone marrow, lymph nodes and spleen.

The term “biological activity” means all inherent biological properties of a molecule.

A “disease-modifying anti-rheumatic drug” (DMARD) means a drug or agent that modulates, reduces or treats the symptoms and/or progression associated with an immune system disease, including autoimmune diseases (e.g., rheumatic diseases), graft-related disorders and immunoproliferative diseases. The DMARD may be a synthetic DMARD (e.g., a conventional synthetic disease modifying antirheumatic drug) or a biologic DMARD. For example, the DMARD used may be a methotrexate, a sulfasalazine (Azulfidine), a cyclosporine (Neoral®, Sandimmune®), a leflunomide (Arava®), a hydroxychloroquine (Plaquenil®), a Azathioprine (Imuran®), or a combination thereof. In various embodiments, a DMARD is used to treat or control progression, joint deterioration, and/or disability associated with an autoimmune disorder, e.g., RA.

The term “polypeptide” means any polymeric chain of amino acids and encompasses native or artificial proteins, polypeptide analogs or variants of a protein sequence, or fragments thereof, unless otherwise contradicted by context. A polypeptide may be monomeric or polymeric. For an antigenic polypeptide, a fragment of a polypeptide optionally contains at least one contiguous or nonlinear epitope of a polypeptide. The precise boundaries of the at least one epitope fragment can be confirmed using ordinary skill in the art.

The term “variant” means a polypeptide that differs from a given polypeptide in amino acid sequence by the addition, deletion, or conservative substitution of amino acids, but that retains the biological activity of the given polypeptide (e.g., a variant TNFα can compete with anti-TNFα antibody for binding to TNF). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al. (1982) J. Mol. Biol. 157:105-132). The hydrophilicity of amino acids also can be used to identify substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” encompasses a polypeptide or fragment thereof that has been differentially processed, such as by proteolysis, phosphorylation, or other post-translational modification, yet retains its biological activity or antigen reactivity, e.g., the ability to bind to TNFα and IL-17. The term “variant” encompasses fragments of a variant unless otherwise contradicted by context.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a protein or polypeptide that is chemically synthesized or synthesized in a cellular system different from the cell from which it naturally originates is isolated from its naturally associated components. A protein or polypeptide may also be rendered substantially free of naturally associated components by isolation using protein purification techniques well known in the art.

The term “human IL-17” (“hIL-17”) includes a homodimeric protein comprising two 15 kD IL-17A proteins (hIL-17A/A) and a heterodimeric protein comprising a 15 kD IL-17A protein and a 15 kD IL-17F protein (“hIL-17A/F”). The amino acid sequences of hIL-17A and hIL-17F are shown in Table 1. The term “hIL-17” includes recombinant hIL-17 (rhIL-17), which can be prepared by standard recombinant expression methods.

Human IL-17A

(SEQ ID NO: 31)

GITIPRNPGCPNSEDKNFPRTVMVNLNIHNRNTNTNPKRSSDYYNRSTSP
WNLHRNEDPERYPSVIWEAKCRHLGCINADGNVDYHMNSVPIQQEILVLR
REPPHCPNSFRLEKILVSVGCTCVTPIVHHVA
Human IL-17F

(SEQ ID NO: 32)

RKIPKVGHTFFQKPESCPPVPGGSMKLDIGIINENQRVSMSRNIESRSTS
PWNYTVTWDPNRYPSEVVQAQCRNLGCINAQGKEDISMNSVPIQQETLVV
RRKHQGCSVSFQLEKVLVTVGCTCVTPVIHHVQ

The phrase “IL-17/TNF-α binding protein” means a bispecific binding protein (e.g., DVD-Ig protein) that binds IL-17 and TNF-α. The relative positions of the TNF-α binding region and IL-17 binding region within the bispecific binding protein are not fixed (e.g., VD1 or VD2 of the DVD-Ig protein) unless specifically specified herein.

The term “human TNF-α” (“hTNF-α”, or simply “hTNF”) means a 17 kD secreted form and a 26 kD membrane associated form of a human cytokine, the biologically active form of which is composed of a trimer of noncovalently bound 17 kD molecules. The structure of hTNFα is described further in, for example, Pennica et al. (1984) Nature 312:724-729; Davis et al. (1987) Biochem. 26:1322-1326; and Jones et al. (1989) Nature 338:225-228. The term hTNF-α includes recombinant human TNFα (“rhTNF-α”). The amino acid sequence of hTNFα is shown below:

Human TNF-α

(SEQ ID NO.: 33)

MSTESMIRDVELAEEALPKKTGGPQGSRRCLFLSLFSFLIVAGATTLFCL
LHFGVIGPQREEFPRDLSLISPLAQAVRSSSRTPSDKPVAHVVANPQAEG
QLQWLNRRANALLANGVELRDNQLVVPSEGLYLIYSQVLFKGQGCPSTHV
LLTHTISRIAVSYQTKVNLLSAIKSPCQRETPEGAEAKPWYEPIYLGGVF
QLEKGDRLSAEINRPDYLDFAESGQVYFGIIAL

The terms “specific binding” or “specifically binding”, in reference to the interaction of an antibody, a protein, or a peptide with a second chemical species, mean that the interaction is dependent upon the presence of a particular structure (e.g., an antigenic determinant or epitope) on the chemical species. If an antibody is specific for epitope “A”, in the presence of a molecule containing epitope A (or free, unlabeled epitope A) in which “A” is labeled, the antibody reduces the amount of labeled A bound to the antibody. “Specific binding partner” is a member of a specific binding pair. The term “specific binding pair” comprises two different molecules, which specifically bind to each other through chemical or physical means (e.g., an antigen (or fragment thereof) and an antibody (or antigenically reactive fragment thereof)). Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog Immunoreactive specific binding members include antigens, antigen fragments, and antibodies, including monoclonal and polyclonal antibodies as well as complexes, fragments, and variants (including fragments of variants) thereof, whether isolated or recombinantly produced. The terms “specific” and “specificity” in the context of an interaction between members of a specific binding pair refer to the selective reactivity of the interaction.

The term “human antibody” includes antibodies having variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” means human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “CDR” means the complementarity determining region within antibody variable sequences. There are three CDRs in each of the variable regions of the heavy chain and the light chain, which are designated CDR1, CDR2, and CDR3, for each of the variable regions. The term “CDR set” means a group of three CDRs that occur in a single variable region (i.e., VH or VL) of an antigen binding site. The exact boundaries of these CDRs have been defined differently according to different systems. The system described by Kabat (Kabat et al. (1987, 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196: 901-917 and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2, and L3 or H1, H2, and H3, where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139 and MacCallum (1996) J. Mol. Biol. 262(5): 732-745). Still other CDR boundary definitions may not strictly follow one of the above systems, but nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The terms “Kabat numbering,” “Kabat definition,” and “Kabat labeling” mean a system of numbering amino acid residues which are more variable (i.e., hypervariable) than other amino acid residues in the heavy and light chain variable regions of an antibody, or an antigen binding portion thereof (Kabat et al. (1971) Ann NY Acad. Sci. 190: 382-391 and Kabat et al. (1991) “Sequences of Proteins of Immunological Interest, Fifth Edition”, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The growth and analysis of extensive public databases of amino acid sequences of variable heavy and light regions over the past twenty years have led to the understanding of the typical boundaries between framework regions (FR) and CDR sequences within variable region sequences and enabled persons skilled in this art to accurately determine the CDRs according to Kabat numbering, Chothia numbering, or other systems. See, e.g., Martin, “Protein Sequence and Structure Analysis of Antibody Variable Domains, “In Kontermann and Dübel, eds., Antibody Engineering (Springer-Verlag, Berlin, 2001), chapter 31, pages 432-433. A useful method of determining the amino acid sequences of Kabat CDRs within the amino acid sequences of variable heavy (VH) and variable light (VL) regions is provided below:

To identify a CDR-L1 amino acid sequence:

Starts approximately 24 amino acid residues from the amino terminus of the VL region;

Residue before the CDR-L1 sequence is always cysteine (C); Residue after the CDR-L1 sequence is always a tryptophan (W) residue, typically Trp-Tyr-Gln (W-Y-Q), but also Trp-Leu-Gln (W-L-Q), Trp-Phe-Gln (W-F-Q), and Trp-Tyr-Leu (W-Y-L);

Length is typically 10 to 17 amino acid residues.

To identify a CDR-L2 amino acid sequence:

Starts always 16 residues after the end of CDR-L1;

Residues before the CDR-L2 sequence are generally Ile-Tyr (I-Y), but also Val-Tyr (V-Y), Ile-Lys (I-K), and Ile-Phe (I-F);

Length is always 7 amino acid residues.

To identify a CDR-L3 amino acid sequence:

Starts always 33 amino acids after the end of CDR-L2;

Residue before the CDR-L3 amino acid sequence is always a cysteine (C);

Residues after the CDR-L3 sequence are always Phe-Gly-X-Gly (F-G-X-G) (SEQ ID NO:34), where X is any amino acid;

Length is typically 7 to 11 amino acid residues.

To identify a CDR-H1 amino acid sequence:

Starts approximately 31 amino acid residues from amino terminus of VH region and always 9 residues after a cysteine (C);

Residues before the CDR-H1 sequence are always Cys-X-X-X-X-X-X-X-X (SEQ ID NO:35), where X is any amino acid;

Residue after CDR-H1 sequence is always a Trp (W), typically Trp-Val (W-V), but also Trp-Ile (W-I), and Trp-Ala (W-A);

Length is typically 5 to 7 amino acid residues.

To identify a CDR-H2 amino acid sequence:

Starts always 15 amino acid residues after the end of CDR-H1;

Residues before CDR-H2 sequence are typically Leu-Glu-Trp-Ile-Gly (L-E-W-I-G) (SEQ ID NO:36), but other variations also;

Residues after CDR-H2 sequence are Lys/Arg-Leu/Ile/Val/Phe/Thr/Ala-Thr/Ser/Ile/Ala (K/R-L/I/V/F/T/A-T/S/I/A);

Length is typically 16 to 19 amino acid residues.

To identify a CDR-H3 amino acid sequence:

Starts always 33 amino acid residues after the end of CDR-H2 and always 3 after a cysteine (C)′

Residues before the CDR-H3 sequence are always Cys-X-X (C-X-X), where X is any amino acid, typically Cys-Ala-Arg (C-A-R);

Residues after the CDR-H3 sequence are always Trp-Gly-X-Gly (W-G-X-G) (SEQ ID NO:37), where X is any amino acid;

Length is typically 3 to 25 amino acid residues.

With respect to constructing DVD-Ig or other binding protein molecules, the term “linker” means a single amino acid or a polypeptide comprising two or more amino acid residues joined by peptide bonds (“linker polypeptide”) used to link one or more antigen binding portions. Such linker polypeptides are well known in the art (see, e.g., Holliger et al., (1993) Proc. Natl. Acad. Sci. USA, 90: 6444-6448; Poljak (1994) Structure, 2: 1121-1123). Exemplary linkers include, but are not limited to, GGGGSG (SEQ ID NO:38), GGSGG (SEQ ID NO:39), GGGGSGGGGS (SEQ ID NO:40), GGSGGGGSG (SEQ ID NO:41), GGSGGGGSGS (SEQ ID NO:42), GGSGGGGSGGGGS (SEQ ID NO:43), GGGGSGGGGSGGGG (SEQ ID NO:44), GGGGSGGGGSGGGGS (SEQ ID NO:45), ASTKGP (SEQ ID NO:46), ASTKGPSVFPLAP (SEQ ID NO:47), TVAAP (SEQ ID NO:48), RTVAAP (SEQ ID NO:49), TVAAPSVFIFPP (SEQ ID NO:50), RTVAAPSVFIFPP (SEQ ID NO:51), AKTTPKLEEGEFSEAR (SEQ ID NO:52), AKTTPKLEEGEFSEARV (SEQ ID NO:53), AKTTPKLGG (SEQ ID NO:54), SAKTTPKLGG (SEQ ID NO:55), SAKTTP (SEQ ID NO:56), RADAAP (SEQ ID NO:57), RADAAPTVS (SEQ ID NO:58), RADAAAAGGPGS (SEQ ID NO:59), RADAAAAGGGGSGGGGSGGGGSGGGGS (SEQ ID NO:60), SAKTTPKLEEGEFSEARV (SEQ ID NO:61), ADAAP (SEQ ID NO:62), ADAAPTVSIFPP (SEQ ID NO:63), QPKAAP (SEQ ID NO:64), QPKAAPSVTLFPP (SEQ ID NO:65), AKTTPP (SEQ ID NO:66), AKTTPPSVTPLAP (SEQ ID NO:67), akttap (SEQ ID NO:68), AKTTAPSVYPLAP (SEQ ID NO:69), GENKVEYAPALMALS (SEQ ID NO:70), GPAKELTPLKEAKVS (SEQ ID NO:71), GHEAAAVMQVQYPAS (SEQ ID NO:72), GSGSGNGS (SEQ ID NO: 81), GSGSGSGS (SEQ ID NO: 82), GGSGSGSG (SEQ ID NO: 83), GGSGSG (SEQ ID NO: 84), GGSG (SEQ ID NO: 85), GGSGNGSG (SEQ ID NO: 86); GGNGSGSG (SEQ ID NO: 87), GGNGSG (SEQ ID NO: 88), GSGS (SEQ ID NO: 89), AND GSG (SEQ ID NO: 90).

The term “neutralizing” mean to render inactive activity, e.g., the biological activity of an antigen (e.g., the cytokines TNFα and IL-17) when a binding protein specifically binds the antigen. Preferably, a neutralizing binding protein described herein binds to human TNFα and/or human IL-17 resulting in the inhibition of a biological activity of the cytokines. Preferably, the neutralizing binding protein binds TNFα and IL-17 and reduces a biologically activity of TNFα and IL-17 by at least about 20%, 30%, 40%, 50%, 60%, 70%, 80%, 85%, 90%, 95%, or more Inhibition of a biological activity of TNFα and IL-17 by a neutralizing binding protein can be assessed by measuring one or more indicators of TNFα and IL-17 biological activity well known in the art.

The term “activity” includes activities such as the binding specificity/affinity of an antibody for an antigen, for example, an anti-TNFα and/or anti-IL-17 (e.g., hTNFα and hIL-17) antibody that binds to TNFα and/or IL-17.

The term “epitope” means a polypeptide determinant capable of specific binding to an immunoglobulin or T-cell receptor. In certain embodiments, epitope determinants include chemically active surface groupings of molecules such as amino acids, sugar side chains, phosphoryl or sulfonyl groups, and, in certain embodiments, may have specific three dimensional structural characteristics and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. Antibodies are said to bind to the same epitope if the antibodies cross-compete (one prevents the binding or modulating effect of the other). In addition, structural definitions of epitopes (overlapping, similar, identical) are informative, but functional definitions are often more relevant as they encompass structural (binding) and functional (modulation, competition) parameters.

The term “percent identity” means a quantitative measurement of the similarity between two sequences (complete amino acid sequence or a portion thereof). Calculations of sequence identity between sequences are known by those in the art. For example, to determine the percent identity of two amino acid sequences, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences. For example, percent identity can about 30%, 35%, 40%, 45%, 50%, 55%, 60%, 65%, 70%, 75%, 80%, 85%, 90%, 95%, 96%, 98%, 99%, or 99% or more.

The comparison of sequences and determination of percent identity between two sequences are accomplished using a mathematical algorithm. Percent identity between two amino acid sequences is determined using an alignment software program using the default parameters. Suitable programs include, for example, CLUSTAL W (see Thompson et al. (1994) Nucl. Acids Res. 22: 4673-4680) or CLUSTAL X.

The term “substantially identical” in reference to amino acid sequences means a first amino acid sequence that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 30%, 35%, 40%, 45%, 50%, 55%, 60%, 65%, 70%, 75%, 80%, 85%, 90%, 95%, 96%, 98%, 99%, or 99% or more identity to a DVD-Ig binding protein described herein (e.g., a DVD-Ig binding protein comprising described herein or a biomarker described herein).

The term “surface plasmon resonance” means an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson et al. (1993) Ann. Biol. Clin. 51: 19-26; Jönsson et al. (1991), BioTechniques 11: 620-627; Johnsson et al. (1995) J. Mol. Recognit. 8: 125-131; and Johnsson et al. (1991) Anal. Biochem. 198: 268-277.

The terms “Kon,” “Kon,” and “kon” mean the on rate constant for association or “association rate constant,” of a binding protein (e.g., an antibody) to an antigen to form an association complex, e.g., antibody/antigen complex, as is known in the art. The term “Kon” also is known by the terms “association rate constant” or “ka”. This value indicates the binding rate of an antibody to its target antigen or the rate of complex formation between an antibody and antigen as is shown by the equation below:

Antibody(“Ab”)+Antigen(“Ag”)→Ab−Ag

The terms “Koff,” “Koff,” and “koff” mean the off rate constant for dissociation, or “dissociation rate constant,” of a binding protein (e.g., an antibody) from an association complex (e.g., an antibody/antigen complex) as is known in the art. This value indicates the dissociation rate of an antibody from its target antigen or separation of Ab-Ag complex over time into free antibody and antigen as shown by the equation below:

Ab+Ag→Ab−Ag

The terms “K_{D}” and “Kd”, and the “equilibrium dissociation constant,” and mean o the value obtained in a titration measurement at equilibrium, or by dividing the dissociation rate constant (Koff) by the association rate constant (Kon). The association rate constant (Kon), the dissociation rate constant (Koff), and the equilibrium dissociation constant (K are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used. Additionally, a KinExA® (Kinetic Exclusion Assay) assay, available from Sapidyne Instruments (Boise, Id.) can also be used.

The terms “AUC” and “area under the curve” mean the area under the plasma drug concentration-time curve, and reflects the actual body exposure to drug after administration of a dose of the drug. AUC is typically related to clearance. A higher clearance rate is related to a smaller AUC, and a lower clearance rate is related to a larger AUC value. The AUC higher values represent slower clearance rates.

The term “volume of distribution” means the theoretical volume of fluid into which the total drug administered would have to be diluted to produce the concentration in plasma. Calculating the volume of distribution may in various embodiments involve the quantification of the distribution of a drug, e.g., a TNFα/IL-17 DVD-Ig binding protein, or antigen-binding portion thereof, between plasma and the rest of the body after dosing. The volume of distribution is the theoretical volume in which the total amount of drug would need to be uniformly distributed in order to produce the desired blood concentration of the drug.

The terms “half-life” and “T½” mean the time for half of a drug's concentration or activity (e.g., pharmacologic or physiologic) to be measurable compared to a previously measured peak concentration or activity. In various embodiments, the quantification of the half-life may involve determining the time taken for half of the concentration or activity a dose of a drug to be measurable, e.g., in the blood, or other body fluid, in a subject or same over time. For example, the half-life may involve the time taken for half of the dose to be eliminated, excreted or metabolized.

The term “Cmax” means the peak concentration that a drug is observed, quantified or measured in a specified fluid or sample after the drug has been administrated. In various embodiments, determining the Cmax involves in part quantification of the maximum or peak serum or plasma concentration of a drug/therapeutic agent observed in a sample from a subject administered the drug.

The term “bioavailability” means the degree to which a drug is absorbed or becomes available to cells or tissue after administration of the drug. For example, bioavailability in certain embodiments involves quantification of the fraction or percent of a dose which is absorbed and enters the systemic circulation after administration of a given dosage form. See International Publication No. WO2013078135, which is incorporated by reference herein in its entirety.

The terms “label” and “detectable label” mean a moiety attached to a specific binding partner, such as an antibody or an analyte, e.g., to render the reaction between two specific binding partners (specific binding pair) detectable. The specific binding partner so labeled is referred to as “detectably labeled”. Thus, the term “labeled binding protein” means a protein with a label incorporated that provides for the identification of the binding protein or the ligand to which it binds. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin or streptavidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹TC, ¹¹¹In, ¹²⁵I, ¹³¹I, ¹⁷⁷Lu, ¹⁶⁶Ho, or ¹⁵³Sm), chromogens, fluorescent labels (e.g., FITC, rhodamine, lanthanide phosphors), enzymatic labels (e.g., horseradish peroxidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), and magnetic agents (e.g., gadolinium chelates). Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety. Use of the term “detectably labeled” is intended to encompass the latter type of detectable labeling.

The term “binding protein conjugate” means a binding protein that is chemically linked to a second chemical moiety, such as a therapeutic or cytotoxic agent.

The term “agent” means a chemical compound, a mixture of chemical compounds, a biological macromolecule, or an extract made from biological materials. The therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, a binding protein conjugate may be a detectably labeled antibody, which is used as the detection antibody.

The term “polynucleotide” means a polymer of two or more nucleotides, e.g., ribonucleotides or deoxynucleotides or a modified form of nucleotide. The term includes single and double stranded forms of DNA.

The term “isolated polynucleotide” means a polynucleotide (e.g., of genomic, cDNA, or synthetic origin, or some combination thereof) that, by virtue of its origin, is not associated with all or a portion of a polynucleotide with which the polynucleotide is found in nature; is operably linked to a polynucleotide that it is not linked to in nature; or does not occur in nature as part of a larger sequence.

The term “vector” means a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional nucleic acid segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked (“recombinant expression vectors” or “expression vectors”). In general, expression vectors are often in the form of plasmids. Vectors may also be viral vectors (e.g., replication defective retroviruses, adenoviruses and adeno-associated viruses).

Standard techniques may be used for recombinant DNA, oligonucleotide synthesis, tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual, 2nd ed. (Cold Spring Harbor Laboratory Press, Cold Spring Harbor, N. Y., 1989).

The term “modulator” means a compound capable of changing or altering an activity or function of a molecule of interest (e.g., the biological activity of hTNFα and hIL-17). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in PCT Publication No. WO01/83525.

The term “agonist” means a modulator that, when contacted with a molecule of interest, causes an increase in the magnitude of a certain activity or function of the molecule compared to the magnitude of the activity or function observed in the absence of the agonist. Particular agonists of interest may include, but are not limited to, TNFα and IL-17 polypeptides, nucleic acids, carbohydrates, or any other molecule that binds to hTNFα and hIL-17.

The terms “antagonist” and “inhibitor” mean a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of human TNFα and IL-17. Antagonists and inhibitors of human TNFα and IL-17 may include, but are not limited to, proteins, nucleic acids, carbohydrates, or any other molecules, which bind to human TNFα and IL-17.

The term “effective amount” means the amount of a therapy that is sufficient to reduce or ameliorate the severity and/or duration of a disorder or one or more symptoms thereof; prevent the advancement of a disorder; cause regression of a disorder; prevent the recurrence, development, onset, or progression of one pr more symptoms associated with a disorder; detect a disorder; or enhance or improve the prophylactic or therapeutic effect(s) of another therapy (e.g., prophylactic or therapeutic agent).

The term “multivalent binding protein” denotes a binding protein comprising two or more antigen binding sites. A multivalent binding protein is preferably engineered to have three or more antigen binding sites, and is generally not a naturally occurring antibody. The term “multispecific binding protein” refers to a binding protein capable of binding two or more related or unrelated targets. “Dual variable domain” (“DVD”) binding proteins of the invention comprise two or more antigen binding sites and are tetravalent or multivalent binding proteins. DVDs may be monospecific, i.e., capable of binding one antigen, or multispecific, i.e., capable of binding two or more antigens. A DVD binding protein comprising two heavy chain DVD polypeptides and two light chain DVD polypeptides is referred to as a “DVD immunoglobulin” or “DVD-Ig”. Each half of a DVD-Ig comprises a heavy chain DVD polypeptide and a light chain DVD polypeptide, and two or more antigen binding sites. Each binding site comprises a heavy chain variable domain and a light chain variable domain with a total of six CDRs involved in antigen binding per antigen binding site. See U.S. Pat. Nos. 7,612,181; 8,258,268, and 8,779,101.

Multivalient binding proteins in various embodiments include bispecific molecules which can be generated using a number of different methods (Spiess et al., 2015 Molecular Immunology pii: S0161-5890, which is incorporated by reference in its entirety). In various embodiments, bispecific molecues comprise Triomab quadroma bispecifics/removab bispecifics, bispecific T cell engagers, tetravalent bispecific tandem diabodies, crossMabs, DART™s, innovative multimers, DutaMabs, asymmetric bispecific antibodies, two-in-one antibodies, Fabsc antibodies, asymmetric bispecific IgG4s, VHHs/Nanobodies™, cross-over dual variable immunoglobulins, biclonics and the like. Multivalient binding proteins in various embodiments include full-length antibodies that are generated by quadroma technology (see Milstein and Cuello, Nature, 305: 537-540 (1983)), by chemical conjugation of two different monoclonal antibodies (see Staerz et al., Nature, 314: 628-631 (1985)), or by knob-into-hole or similar approaches which introduces mutations in the Fc region (see Holliger et al., Proc. Natl. Acad. Sci. USA, 90(14): 6444-6448 (1993)), resulting in multiple different immunoglobulin species of which only one is the functional bispecific antibody.

For example, a TNF/IL-17 bispecific can be prepared using any number of formats and techniques: fusion protein, bispecific nanobody, a cross mab, diabody, and DVD-Ig formats (See European patent application EP 2597102A1, international application numbers 2012156219 and WO2014137961, Fischer et al. 2015 Arthritis Rheumatol 67:51-62. doi:10.1002/art.38896, U.S. publication number 20140079705, and U.S. Pat. No. 8,779,101, each of which is incorporated by reference in its entirety). The terms “single chain dual variable domain immunoglobulin protein” or “scDVD-Ig protein” or scFvDVDIg protein” refer to the antigen binding fragment of a DVD molecule that is analogous to an antibody single chain Fv fragment. scDVD-Ig proteins are described in U.S. Ser. Nos. 61/746,659; 14/141,498; and 14/141,500, incorporated herein by reference in their entireties. scDVD-Ig proteins are generally of the formula VH1-(X1)n-VH2-X2-VL1-(X3)n-VL2, where VH1 is a first antibody heavy chain variable domain, X1 is a linker with the proviso that it is not a constant domain, VH2 is a second antibody heavy chain variable domain, X2 is a linker, VL1 is a first antibody light chain variable domain, X3 is a linker with the proviso that it is not a constant domain, VL2 is a second antibody light chain variable domain, and n is 0 or 1, where the VH1 and VL1, and the VH2 and VL2 respectively combine to form two functional antigen binding sites.

The terms “DVD-Fab” or fDVD-Ig protein” refer to the antigen binding fragment of a DVD-Ig molecule that is analogous to an antibody Fab fragment. fDVD-Ig proteins are described in U.S. Ser. Nos. 61/746,663; 14/141,498; and 14/141,501, incorporated herein by reference in their entireties. In certain embodiments, fDVD-Ig proteins include a first polypeptide chain having the general formula VH1-(X1)n-VH2-C-(X2)n, wherein VH1 is a first heavy chain variable domain, X1 is a linker with the proviso that it is not a constant domain, VH2 is a second heavy chain variable domain, C is a heavy chain constant domain, X2 is a cell surface protein, and n is 0 or 1, and wherein the amino acid sequences of VH1, VH2 and/or X1 independently vary within the library. In certain embodiments, the fDVD-Ig proteins also include a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional antigen binding sites. In certain embodiments, the first and second polypeptide chains combine to form an fDVD-Ig protein.

The terms “receptor DVD-Ig protein” constructs, or “rDVD-Ig protein” refer to DVD-Ig constructs comprising at least one receptor-like binding domain. rDVD-Ig proteins are described in U.S. Ser. Nos. 61/746,616; and 14/141,499, incorporated herein by reference in their entireties. Variable domains of the rDVD-Ig molecule may include one immunoglobulin variable domain and one non-immunoglobulin variable domain such as a ligand binding domain of a receptor, or an active domain of an enzyme. rDVD-Ig molecules may also comprise two or more non-Ig domains (see PCT Publication No. WO 02/02773). In rDVD-Ig protein at least one of the variable domains comprises a ligand binding domain of a receptor, or receptor domain (RD).

The term “receptor domain” (RD), or receptor binding domain refers to the portion of a cell surface receptor, cytoplasmic receptor, nuclear receptor, or soluble receptor that functions to bind one or more receptor ligands or signaling molecules (e.g., toxins, hormones, neurotransmitters, cytokines, growth factors, or cell recognition molecules).

The terms multi-specific and multivalent IgG-like molecules or “pDVD-Ig” proteins are capable of binding two or more proteins (e.g., antigens). pDVD-Ig proteins are described in U.S. Ser. Nos. 61/746,617; 14/141,498; and 14/141,502, incorporated herein by reference in their entireties. In certain embodiments, pDVD-Ig proteins are disclosed which are generated by specifically modifying and adapting several concepts. These concepts include but are not limited to: (1) forming Fc heterodimer using CH3 “knobs-into-holes” design, (2) reducing light chain missing pairing by using CH1/CL cross-over, and (3) pairing two separate half IgG molecules at protein production stage using “reduction then oxidation” approach.

In certain embodiments, the binding protein of the invention is a “half-DVD-Ig” proteins derived from a DVD-Ig protein. The half-DVD-Ig protein preferably does not promote cross-linking observed with naturally occurring antibodies which can result in antigen clustering and undesirable activities. See U.S. Patent Publication No. 20120201746, published Aug. 9, 2012, and International Publication No. WO/2012/088302, published Jun. 28, 2012, each of which is incorporated by reference herein in its entirety.

In one embodiment, a polyvalent DVD-Ig (pDVD-Ig) construct may be created by combining two halves of different DVD-Ig molecules, or a half DVD-Ig protein and half IgG molecule. A pDVD-Ig construct may be expressed from four unique constructs to create a monovalent, multi-specific molecules through the use of heavy chain CH3 knobs-into-holes design. In another embodiment, a pDVD-Ig construct may contain two distinct light chains, and may utilize structural modifications on the Fc of one arm to ensure the proper pairing of the light chains with their respective heavy chains. In one aspect, the heavy chain constant region CH1 may be swapped with a light chain constant region hCk on one Fab. In another aspect, an entire light chain variable region, plus hCk, may be swapped with a heavy chain variable region, plus CH1. pDVD-Ig construct vectors that accommodate these unique structural requirements are also disclosed.

In some embodiments, pDVD-Ig proteins contain four polypeptide chains, namely, first, second, third and fourth polypeptide chains. In one aspect, the first polypeptide chain may contain VD1-(X1)n-VD2-CH-(X2)n, wherein VD1 is a first heavy chain variable domain, VD2 is a second heavy chain variable domain, CH is a heavy chain constant domain, X1 is a linker with the proviso that it is not a constant domain, and X2 is an Fc region. In another aspect, the second polypeptide chain may contain VD1-(X1)n-VD2-CL-(X2)n, wherein VD1 is a first light chain variable domain, VD2 is a second light chain variable domain, CL is a light chain constant domain, X1 is a linker with the proviso that it is not a constant domain, and X2 does not comprise an Fc region. In another aspect, the third polypeptide chain may contain VD3-(X3)n-VD4-CL-(X4)n, wherein VD3 is a third heavy chain variable domain, VD4 is a fourth heavy chain variable domain, CL is a light chain constant domain, X3 is a linker with the proviso that it is not a constant domain, and X4 is an Fc region. In another aspect, the fourth polypeptide chain may contain VD3-(X3)n-VD4-CH-(X4)n, wherein VD3 is a third light chain variable domain, VD4 is a fourth light chain variable domain, CH is a heavy chain constant domain, X3 is a linker with the proviso that it is not a constant domain, and X4 does not comprise an Fc region. In another aspect, n is 0 or 1, and the VD1 domains on the first and second polypeptide chains form one functional binding site for antigen A, the VD2 domains on the first and second polypeptide chains form one functional binding site for antigen B, the VD3 domains on the third and fourth polypeptide chains form one functional binding site for antigen C, and the VD4 domains on the third and fourth polypeptide chains form one functional binding site for antigen D. In one embodiment, antigens A, B, C and D may be the same antigen, or they may each be a different antigen. In another embodiment, antigens A and B are the same antigen, and antigens C and D are the same antigen.

As used herein “monobody DVD-Ig protein” or “mDVD-Ig protein” refers to a class of binding molecules wherein one binding arm has been rendered non-functional. mDVD-Ig proteins are described in U.S. Ser. Nos. 61/746,615; 14/141,498; and 14/141,503, incorporated herein by reference in their entireties. In one aspect, an mDVD-Ig protein possesses only one functional arm capable of binding a ligand. In another aspect, the one functional arm may have one or more binding domains for binding to different ligands. The ligand may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and combinations thereof.

In one embodiment, an mDVD-Ig protein contains four polypeptide chains, wherein two of the four polypeptide chains comprise VDH-(X1)n-C-(X2)n. In one aspect, VDH is a heavy chain variable domain, X1 is a linker with the proviso that it is not CH1, C is a heavy chain constant domain, X2 is an Fc region, and n is 0 or 1. The other two of the four polypeptide chains comprise VDL-(X3)n-C-(X4)n, wherein VDL is a light chain variable domain, X3 is a linker with the proviso that it is not CH1, C is a light chain constant domain, X4 does not comprise an Fc region, and n is 0 or 1. In another aspect, at least one of the four polypeptide chains comprises a mutation located in the variable domain, wherein the mutation inhibits the targeted binding between the specific antigen and the mutant binding domain.

The Fc regions of the two polypeptide chains that have a formula of VDH-(X1)n-C-(X2)n may each contain a mutation, wherein the mutations on the two Fc regions enhance heterodimerization of the two polypeptide chains. In one aspect, knobs-into-holes mutations may be introduced into these Fc regions to achieve heterodimerization of the Fc regions. See Atwell et al. J. Mol. Biol. 1997, 270: 26-35.

A “cross-over DVD-Ig” protein or “coDVD-Ig” protein refers to a DVD-Ig protein wherein the cross-over of variable domains is used to resolve the issue of affinity loss in the inner antigen-binding domains of some DVD-Ig molecules. coDVD-Ig proteins are described in U.S. Ser. Nos. 61/746,619; 14/141,498; and 14/141,504, incorporated herein by reference in their entireties. In certain specific embodiments, coDVD-Ig″ proteins are generated by crossing over light chain and the heavy chain variable domains of a DVD-Ig protein or DVD-Ig-like protein. In another aspect, the length and sequence of the linkers linking the variable domains may be optimized for each format and antibody sequence/structure (frameworks) to achieve desirable properties. The disclosed concept and methodology may also be extended to Ig or Ig-like proteins having more than two antigen binding domains.

A “blood-brain-bather DVD” (bbbDVD-Ig) means a dual variable domain binding protein comprising at least a first and a second binding domain, such that the at least one binding domain specifically binds a target that facilitates entrance or passage of the binding protein across a natural BBB biological barrier. For example, the target is a receptor on vascular endothelial cells of the BBB. In various embodiments the receptor is selected from insulin receptor, transferrin receptor, LRP, melanocortin receptor, nicotinic acetylcholine receptor, VACM-1 receptor, IGFR, EPCR, EGFR, TNFR, Leptin receptor, M6PR, Lipoprotein receptor, NCAM, LIFR, LfR, MRP1, AchR, DTr, Glutathione transporter, SR-B1, MYOF, TFRC, ECE1, LDLR, PVR, CDC50A, SCARF1, MRC1, HLA-DRA, RAMP2, VLDLR, STAB1, TLR9, CXCL16, NTRK1, CD74, DPP4, endothelial growth factor receptors 1, 2 and 3, glucocorticoid receptor, ionotropic glutamate receptor, M3 receptor, aryl hydrocarbon receptor, GLUT-1, inositol-1,4,5-trisphosphate (IP3) receptor, N-methyl-D-aspartate receptor, 51P1, P2Y receptor, TMEM30A, and RAGE. See WO/2014/089209.

The terms “tri-variable binding protein”, “triple variable binding protein”, and “TVD binding protein”, as used herein include molecules that contain three or six antigen binding sites, each of which independently and specifically binds a target antigen. In one embodiment, a TVD binding protein is a TVD-Immunoglobulin (TVD-Ig) binding protein that can bind a triplet of antigens. See U.S. publication number 20120195900.A “cleavable linker DVD-Ig” (clDVD-Ig) molecule means a DVD binding protein that is cleavable by an enzyme. In various embodiments, the the VD1 or VD2 does not bind to its target until a cleavage between the VD1 and VD2 occurs. In various embodiments, contraints in the DVD-Ig are ameliorated or removed by being cleaved. In an embodiment, the DVD-Ig is cleavable by an enzyme between the VD1 (VH1, VL1) and VD2 (VH2, VL2) domains of at least one of a heavy chain and a light chain. In an embodiment, a cleavable linker joins the VD1 and VD2 domain of at least one of a heavy chain and a light chain. See U.S. publication number 20100233079.

A “redirected cytotoxicity DVD-Ig” (rcDVD-Ig) molecule means a binding protein and described in U.S. publication numbers 20140205613 and 20150023949.

Various aspects of the invention are described in further detail in the following subsections.

I. Prediction of Responsiveness to a Combination Therapy Comprising an Anti-TNF Treatment and an Anti-IL-17 Treatment in Subjects with Inflammatory Disease, and Related Methods.

In various aspects, the invention provides a method for determining whether a subject having an inflammatory disease will respond to treatment with a combination therapy comprising an anti-TNF treatment and an anti-IL-17 treatment. The method includes the steps of determining a level of expression of at least one of a marker (e.g., LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof) in a sample obtained from the subject; and comparing the level of expression of the marker(s) to the level of expression of a control marker. A higher level of expression of at least one of the markers (e.g., LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR), as compared to the level of expression of the control marker, indicates that the combination therapy will be effective in treating the subject. Alternatively, a lower level of expression of at least one of the markers (e.g., LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR) after a combination therapy comprising an anti-TNF treatment and an anti-IL-17 treatment, as compared to the level of expression of the control marker before treatment with the combination therapy, indicates that the combination therapy will be effective in treating the subject.

In another aspect, the present invention provides a method of determining whether a subject having an inflammatory disease will respond to treatment with a combination therapy comprising an anti-TNF treatment and an anti-IL-17 treatment. The method includes the steps of processing a sample obtained from the subject to allow the determination of a level of expression of at least one of a marker and comparing the level of expression of the marker(s) to the level of expression of a control marker, e.g., a normal or disease standard or range of laboratory values). A higher level of expression of at least one of the markers, as compared to the level of expression of the control marker, indicates that the combination therapy will be effective in treating the subject. Alternatively, a lower level of expression of at least one of the markers after a combination therapy comprising an anti-TNF treatment and an anti-IL-17 treatment, as compared to the level of expression of the control marker, indicates that the combination therapy will be effective in treating the subject.

In still another aspect, the present invention provides a method of treating a subject having an inflammatory disease with a combination therapy comprising an anti-TNF treatment and an anti-IL-17 treatment. The method includes the steps of selecting a subject exhibiting a higher level of expression of at least one of a marker as compared to a level of expression of a control marker and administering a therapeutically effective amount of the combination therapy to the subject. Alternatively, a lower level of expression of at least one of the marker after a combination therapy comprising an anti-TNF treatment and an anti-IL-17 treatment, as compared to the level of expression of the control marker, indicates that the combination therapy will be effective in treating the subject.

In still another aspect, the present invention provides a method of contraindicating a subject having an inflammatory disease from a combination therapy comprising an anti-TNF treatment and an anti-IL-17 treatment. The method includes the steps of selecting a subject exhibiting a lower level of expression of at least one of a LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR marker as compared to a level of expression of a control marker, or a normal range of laboratory values.

In yet another aspect, the present invention provides a method for monitoring the effectiveness of a treatment with a combination therapy comprising an anti-TNF treatment and an anti-IL-17 treatment. The method includes the steps of determining a level of expression of at least one of a marker in a sample obtained from a subject following administering a therapeutically effective amount of the combination therapy to the subject and comparing the level of expression of the marker(s) to a level of expression of a control marker, e.g., a normal range of laboratory values. A lower level of expression of at least one of the markers, as compared to the level of expression of the control marker, indicates that the combination therapy has been effective in treating the subject.

In another aspect, the present invention provides a method of selecting a subject for participation in a clinical trial for a combination therapy comprising an anti-TNF treatment and an anti-IL-17 treatment for the treatment of an inflammatory disease. The method includes the steps of determining a level of expression of at least one of a marker in a sample obtained from the subject and comparing the level of expression of the marker(s) to a level of expression of a control marker. A higher level of expression of at least one of the markers, as compared to the level of expression of the control marker, indicates that the subject is suitable for participation in the clinical trial. Alternatively, a lower level of expression of at least one of the CSF markers after a combination therapy comprising an anti-TNF treatment and an anti-IL-17 treatment, as compared to the level of expression of the control marker, indicates that the combination therapy will be effective in treating the subject in the clinical trial.

In still another aspect, the present invention provides a method for identifying a combination therapy comprising an anti-TNF treatment and an anti-IL-17 treatment suitable for treating a subject having an inflammatory disease. The method includes the steps of determining a level of expression of at least one of the and/or G-CSF markers in a sample obtained from the subject and comparing the level of expression of the marker(s) to a level of expression of a control marker. A higher level of expression of at least one of the markers, as compared to the level of expression of the control marker, indicates that the combination therapy will be effective in treating the subject. The method can include testing a plurality of different combination therapies. Alternatively, a lower level of expression of at least one of the markers after the combination therapy is administered to the subject, as compared to the level of expression of the control marker pre-treatment with the combination therapy, indicates that the combination therapy will be effective in treating the subject.

In yet another aspect, the present invention provides a method of determining whether a subject having an inflammatory disease will respond to treatment with a combination therapy comprising an anti-TNFα antibody and an anti-IL-17 antibody. The method includes the steps of determining a level of expression of at least one of a marker in a sample obtained from the subject using a reagent that interacts with at least one of the markers and transforms the sample such that at least one of the markers can be detected and comparing the level of expression of at least one of the markers to the level of expression of a control marker. A higher level of expression of at least one of the F markers, as compared to the level of expression of the control marker, e.g., a normal range of laboratory values, indicates that the combination therapy will be effective in treating the subject. Alternatively, a lower level of expression of at least one of the markers after a combination therapy comprising an anti-TNF treatment and an anti-IL-17 treatment has been administered, as compared to the level of expression of the control marker, indicates that the combination therapy will be effective in treating the subject.

In yet another aspect, the present invention provides a method of determining whether a candidate substance will be effective in treating an inflammatory disease. The method includes the steps of administering the candidate substance to a subject suffering from an inflammatory disease, determining a level of expression of at least one of a marker in a sample obtained from the subject using a reagent that interacts with at least one of the markers and transforms the sample such that at least one of the markers can be detected and comparing the level of expression of at least one of the CSF markers to the level of expression of a control marker. A lower level of expression of at least one of the markers, as compared to the level of expression of the control marker, e.g., levels from one or more subjects with the inflammatory disease who have not received the candidate substance, indicates that the candidate substance will be effective in treating the inflammatory disease. Alternatively, a higher level of expression of at least one of the markers after administration of the candidate compound, as compared to the level of expression of the control marker, indicates that the combination therapy will be ineffective in treating the inflammatory disease.

In another aspect, the present invention provides a method of determining whether an inflammatory disease will respond to treatment with a combination therapy comprising an anti-TNF treatment and an anti-IL-17 treatment. The method includes the steps of processing a sample obtained from a subject suffering from the inflammatory disease such that the sample is transformed, thereby allowing the determination of a level of expression of at least one of a LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof marker and comparing the level of expression of the marker(s) to the level of expression of a control marker, e.g., a normal or disease standard or range of laboratory values). A higher level of expression of at least one of the markers, as compared to the level of expression of the control marker, indicates that the combination therapy will be effective in treating the subject. Alternatively, a lower level of expression of at least one of the markers after a combination therapy comprising an anti-TNF treatment and an anti-IL-17 treatment, as compared to the level of expression of the control marker, indicates that the combination therapy will be effective in treating the subject.

In still yet another aspect, the present invention provides a kit including reagents for determining a level of expression of at least one of a marker in a sample obtained from the subject and a control marker, e.g., a normal range of values. The kit also includes instructions for (i) determining whether the subject will respond to the combination therapy; (ii) monitoring the effectiveness of the combination therapy; (iii) selecting a subject for participation in a clinical trial for the combination therapy; and/or (iv) identifying a combination therapy comprising an anti-TNF treatment and an anti-IL-17 treatment for a subject having an inflammatory disease. Instructions can correspond to any one or more of the aspects described herein.

Any suitable analytical method, can be utilized in the methods of the invention to assess (directly or indirectly) the level of expression of a biomarker in a sample. In an embodiment, a difference is observed between the level of expression of a biomarker, as compared to the control level of expression of the biomarker. In one embodiment, the difference is greater than the limit of detection of the method for determining the expression level of the biomarker. In further embodiments, the difference is greater than or equal to the standard error of the assessment method, e.g., the difference is at least about 2-, about 3-, about 4-, about 5-, about 6-, about 7-, about 8-, about 9-, about 10-, about 15-, about 20-, about 25-, about 100-, about 500- or about 1000-fold greater than the standard error of the assessment method. In an embodiment, the level of expression of the biomarker in a sample as compared to a control level of expression is assessed using parametric or nonparametric descriptive statistics, comparisons, regression analyses, and the like.

In an embodiment, a difference in the level of expression of the biomarker in the sample derived from the subject is detected relative to the control, and the difference is about 5%, about 10%, about 15%, about 20%, about 25%, about 30%, about 40%, about 50%, about 60%, about 70%, about 80%, about 90%, about 100%, about 150%, about 200%, about 250%, about 300%, about 350%, about 400%, about 450%, about 500%, about 600%, about 700%, about 800%, about 900% or about 1000% greater than the expression level of the biomarker in the control sample.

In an embodiment, a difference in the level of expression of the biomarker in the sample derived from the subject is detected relative to the control, and the difference is about 5%, about 10%, about 15%, about 20%, about 25%, about 30%, about 40%, about 50%, about 60%, about 70%, about 80%, or about 90% less than the expression level of the biomarker in the control sample.

The level of expression of a biomarker, for example LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR, in a sample obtained from a subject may be assayed by any of a wide variety of techniques and methods, which transform the biomarker within the sample into a moiety that can be detected and/or quantified. Non-limiting examples of such methods include analyzing the sample using immunological methods for detection of proteins, protein purification methods, protein function or activity assays, nucleic acid hybridization methods, nucleic acid reverse transcription methods, and nucleic acid amplification methods, immunoblotting, Western blotting, Northern blotting, electron microscopy, mass spectrometry, e.g., MALDI-TOF and SELDI-TOF, immunoprecipitations, immunofluorescence, immunohistochemistry, enzyme linked immunosorbent assays (ELISAs), e.g., amplified ELISA, quantitative blood based assays, e.g., serum ELISA, quantitative urine based assays, flow cytometry, Southern hybridizations, array analysis, and the like, and combinations or sub-combinations thereof.

In one embodiment, the level of expression of the biomarker in a sample is determined by detecting a transcribed polynucleotide, or portion thereof, e.g., mRNA, or cDNA, of the biomarker gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, quantitative PCR analysis, RNase protection assays, Northern blotting and in situ hybridization. Other suitable systems for mRNA sample analysis include microarray analysis (e.g., using Affymetrix's microarray system or Illumina's BeadArray Technology).

In one embodiment, the level of expression of the biomarker is determined using a nucleic acid probe. The term “probe”, as used herein, refers to any molecule that is capable of selectively binding to a specific biomarker. Probes can be synthesized by one of skill in the art, or derived from appropriate biological preparations. Probes can be specifically designed to be labeled, by addition or incorporation of a label. Examples of molecules that can be utilized as probes include, but are not limited to, RNA, DNA, proteins, antibodies, and organic molecules.

As indicated above, isolated mRNA can be used in hybridization or amplification assays that include, but are not limited to, Southern or Northern analyses, polymerase chain reaction (PCR) analyses and probe arrays. One method for the determination of mRNA levels involves contacting the isolated mRNA with a nucleic acid molecule (probe) that can hybridize to the biomarker mRNA. The nucleic acid probe can be, for example, a full-length cDNA, or a portion thereof, such as an oligonucleotide of at least about 7, 10, 15, 20, 25, 30, 35, 40, 45, 50, 100, 250 or about 500 nucleotides in length and sufficient to specifically hybridize under appropriate hybridization conditions to the biomarker genomic DNA. In a particular embodiment, the probe will bind the biomarker genomic DNA under stringent conditions. Such stringent conditions, for example, hybridization in 6× sodium chloride/sodium citrate (SSC) at about 45° C., followed by one or more washes in 0.2×SSC, 0.1% SDS at 50-65° C., are known to those skilled in the art and can be found in Current Protocols in Molecular Biology, Ausubel et al., eds., John Wiley & Sons, Inc. (1995), sections 2, 4, and 6, the teachings of which are hereby incorporated by reference herein. Additional stringent conditions can be found in Molecular Cloning: A Laboratory Manual, Sambrook et al., Cold Spring Harbor Press, Cold Spring Harbor, N.Y. (1989), chapters 7, 9, and 11, the teachings of which are hereby incorporated by reference herein.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface, for example, in an Affymetrix gene chip array, and the probe(s) are contacted with mRNA. A skilled artisan can readily adapt mRNA detection methods for use in determining the level of the biomarker mRNA.

The level of expression of the biomarker in a sample can also be determined using methods that involve the use of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules. These approaches are especially useful for the detection of nucleic acid molecules if such molecules are present in very low numbers. In particular aspects of the invention, the level of expression of the biomarker is determined by quantitative fluorogenic RT-PCR (e.g., the TaqMan™ System). Such methods typically utilize pairs of oligonucleotide primers that are specific for the biomarker. Methods for designing oligonucleotide primers specific for a known sequence are well known in the art.

The expression levels of biomarker mRNA can be monitored using a membrane blot (such as used in hybridization analysis such as Northern, Southern, dot, and the like), or microwells, sample tubes, gels, beads or fibers (or any solid support comprising bound nucleic acids). See, for example, U.S. Pat. Nos. 5,770,722; 5,874,219; 5,744,305; 5,677,195; and 5,445,934, the entire contents of which as they relate to these assays are incorporated herein by reference. The determination of biomarker expression level may also comprise using nucleic acid probes in solution.

In one embodiment of the invention, microarrays are used to detect or quantify the level of expression of a biomarker. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, e.g., U.S. Pat. Nos. 6,040,138; 5,800,992; 6,020,135; 6,033,860; and 6,344,316, the entire contents of which as they relate to these assays are incorporated herein by reference. High-density oligonucleotide arrays are particularly useful for determining the gene expression profile for a large number of RNA's in a sample.

Expression of a biomarker can also be assessed at the protein level, using a detection reagent that detects the protein product encoded by the mRNA of the biomarker, directly or indirectly. For example, if an antibody reagent is available that binds specifically to a biomarker protein product to be detected, then such an antibody reagent can be used to detect the expression of the biomarker in a sample from the subject, using techniques, such as immunohistochemistry, ELISA, FACS analysis, and the like.

Other known methods for detecting the biomarker at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, and Western blotting.

Proteins from samples can be isolated using a variety of techniques, including those well known to those of skill in the art. The protein isolation methods employed can, for example, be those described in Harlow and Lane (Harlow and Lane, 1988, Antibodies: A Laboratory Manual, Cold Spring Harbor Laboratory Press, Cold Spring Harbor, N.Y.).

In one embodiment, antibodies, or antibody fragments, are used in methods such as Western blots or immunofluorescence techniques to detect the expressed proteins. Antibodies for determining the expression of the biomarkers of the invention are commercially available.

Anti-CXCL1 antibodies are readily available from a number of commercial suppliers. For example, EMD Millipore: AP1151-100UG, Everest Biotech: EB09637, Lifespan Biosciences: LS-B2843, LS-B2513, LS-C108147, eBioscience: 50-7519-42, 50-7519-41, AbD Serotec: AAM40B, AAM40, AAR22B, Thermo Fisher Scientific, Inc.: PA1-32959, PA1-32924, PA1-20861, Abbiotec: 251349, 12335-1-AP, AP08852PU-N, NovaTeinBio: 63059, Abgent: AT1688a, Aviva Systems Biology: AVARP07032_P050, OASA08635, OAEB00281, United States Biological: C8297-97A, C8298-01B, C8298-01C, Creative Biomart: CAB-1005MH, CAB-3086MH, CAB-115MH, Novus Biologicals: NBP1-61297, NBP1-51486, NBP1-19301, Abnova: H00002919-M01, H00002919-DO1P, H00002919-M03, Fitzgerald: 70R-10502, ProSci: 31-057, 42-129, 42-196.

For example, in one embodiment, the methods of the invention may comprise contacting a sample from the subject with an antibody that specifically binds to CXCL10, CXCL1 and/or G-CSF, forming a complex between the antibody and CXCL1 and/or CLXCL5, adding a detection reagent or antibody that is labeled and reactive with the antibody that binds to CXCL10, CXCL1 and/or G-CSF to detect the complex, washing to remove any unbound detection reagent or antibody, converting the label to the detectable signal and comparing the level of CXCL10, CXCL1 and/or G-CSF measured to a reference level of CXCL10, CXCL1 and/or G-CSF obtained from a control sample.

In one embodiment, the antibody or protein can be immobilized on a solid support for Western blots and immunofluorescence techniques. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

Other standard methods include immunoassay techniques which are well known to one of ordinary skill in the art and may be found in Principles And Practice Of Immunoassay, 2nd Edition, Price and Newman, eds., MacMillan (1997) and Antibodies, A Laboratory Manual, Harlow and Lane, eds., Cold Spring Harbor Laboratory, Ch. 9 (1988).

In one embodiment of the invention, proteomic methods, e.g., mass spectrometry, are used. Mass spectrometry is an analytical technique that consists of ionizing chemical compounds to generate charged molecules (or fragments thereof) and measuring their mass-to-charge ratios. In a typical mass spectrometry procedure, a sample is obtained from a subject, loaded onto the mass spectrometry, and its components (e.g., the biomarker) are ionized by different methods (e.g., by impacting them with an electron beam), resulting in the formation of charged particles (ions). The mass-to-charge ratio of the particles is then calculated from the motion of the ions as they transit through electromagnetic fields.

For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright et al. (2002) Expert Rev. Mol. Diagn. 2:549; Li et al. (2002) Clin Chem 48:1296; Laronga et al. (2003) Dis biomarkers 19:229; Petricoin et al. (2002) 359:572; Adam et al. (2002) Cancer Res 62:3609; Tolson et al. (2004) Lab Invest 84:845; Xiao et al. (2001) Cancer Res 61:6029) can be used to determine the expression level of a biomarker at the protein level.

Furthermore, in vivo techniques for determination of the expression level of the biomarker include introducing into a subject a labeled antibody directed against the biomarker, which binds to and transforms the biomarker into a detectable molecule. As discussed above, the presence, level, or even location of the detectable biomarker in a subject may be detected by standard imaging techniques.

In general, where a difference in the level of expression of a biomarker and the control is to be detected, it is preferable that the difference between the level of expression of the biomarker in a sample from a subject having an inflammatory disease (e.g., rheumatoid arthritis) and being treated with an anti-TNF treatment and an anti-IL-17 treatment, or being considered for such treatment, and the amount of the biomarker in a control sample, is as great as possible. Although this difference can be as small as the limit of detection of the method for determining the level of expression, it is preferred that the difference be greater than the limit of detection of the method or greater than the standard error of the assessment method, and preferably a difference of at least about 2-, about 3-, about 4-, about 5-, about 6-, about 7-, about 8-, about 9-, about 10-, about 15-, about 20-, about 25-, about 100-, about 500-, 1000-fold greater than the standard error of the assessment method. Alternatively, the difference be greater than the limit of detection of the method or greater than the standard error of the assessment method, and preferably a difference of at least about 2-, about 3-, about 4-, about 5-, about 6-, about 7-, about 8-, about 9-, about 10-, about 15-, about 20-, about 25-, about 100-, about 500-, 1000-fold less than the standard error of the assessment method.

Any suitable sample obtained from a subject having an inflammatory disease (e.g., RA) may be used to assess the level of expression, including an increase or a lack of expression, of the biomarker, for example CXCL10, CXCL1 TNFγ, GM-CSFR, G-CSFR, and/or G-CSF. For example, the sample may be any fluid or component thereof, such as a fraction or extract, e.g., blood, plasma, lymph, synovial fluid, cystic fluid, urine, nipple aspirates, or fluids collected from a biopsy, amniotic fluid, aqueous humor, vitreous humor, bile, blood, breast milk, cerebrospinal fluid, cerumen, chyle, cystic fluid, endolymph, feces, gastric acid, gastric juice, mucus, pericardial fluid, perilymph, peritoneal fluid, plasma, pleural fluid, pus, saliva, sebum, semen, sweat, serum, sputum, synovial fluid, joint tissue or fluid, tears, or vaginal secretions obtained from the subject. In a typical situation, the fluid may be blood, or a component thereof, obtained from the subject, including whole blood or components thereof, including, plasma, serum, and blood cells, such as red blood cells, white blood cells and platelets. In another typical situation, the fluid may be synovial fluid, joint tissue or fluid, or any other sample reflective of an inflammatory disease (e.g., rheumatoid arthritis). The sample may also be any tissue or component thereof, connective tissue, lymph tissue or muscle tissue obtained from the subject.

Techniques or methods for obtaining samples from a subject are well known in the art and include, for example, obtaining samples by a mouth swab or a mouth wash; drawing blood; obtaining a biopsy; or obtaining synovial fluid or other sample from a subject suffering from inflammatory disease (e.g., skin, as in the case of psoriasis or psoriatic arthritis). Isolating components of fluid or tissue samples (e.g., cells or RNA or DNA) may be accomplished using a variety of techniques. After the sample is obtained, it may be further processed.

II. Treatment with a Combination Therapy Comprising an Anti-TNF Treatment and an Anti-IL-17 Treatment.

Given the observation that the expression levels of certain markers (e.g., CXCL10, CXCL1 and/or G-CSF) in a subject having inflammatory disease (e.g., RA) influences the responsiveness of the subject to a combination therapy of an anti-TNF treatment and an anti-IL-17 treatment, a skilled artisan can select an appropriate treatment regimen for the subject based on the expression levels of the biomarkers in the subject.

Accordingly, the present invention provides methods for treating a subject having an inflammatory disease with a combination therapy comprising an anti-TNF treatment and an anti-IL-17 treatment.

In an embodiment, the subject may have been previously treated with a monotherapy comprising an anti-TNF treatment or an anti-IL-17 treatment, a combination therapy comprising an anti-TNF treatment and an anti-IL-17 treatment, and/or an alternative therapy. In other embodiments, the subject may be under consideration for treatment with a combination therapy comprising an anti-TNF treatment and an anti-IL-17 treatment for the first time. For example, the level of expression of at least one of a CXCL1 marker and a CXCL5 marker is determined. If the level of expression of at least one of the CXCL1 and CXCL5 biomarker is determined to be higher than a control level of expression, treatment with a combination therapy comprising an anti-TNF treatment and an anti-IL-17 treatment is likely to be efficacious. However, it is not necessary that all of the biomarkers assayed have a high level of expression as compared to the respective control. For example, while certain biomarkers may be present at normal or high levels of expression, treatment with a combination therapy comprising an anti-TNF treatment and an anti-IL-17 treatment, may be indicated when, for example, either CXCL1 or CXCL5 is expressed at a lower level than a control level.

The treatment regimen for a combination therapy comprising an anti-TNF treatment and an anti-IL-17 treatment, that is selected typically includes at least one of the following parameters and more typically includes many or all of the following parameters: the dosage, the formulation, the route of administration and/or the frequency of administration. Selection of the particular parameters of the treatment regimen can be based on known treatment parameters for an anti-TNF therapy and an anti-IL-17 therapy previously established in the art such as those described in the Dosage and Administration protocols set forth in the FDA Approved Label for Adalimumab, infliximab, and secukinumab, the entire contents of which are incorporated herein by reference. Various modifications to dosage, formulation, route of administration and/or frequency of administration can be made based on various factors including, for example, the disease, age, sex, and weight of the patient, as well as the severity or stage of inflammatory disease (e.g., RA) by methods known in the art.

The anti-TNF treatment and the anti-IL-17 treatment may be administered at the same time or at different times. A combination therapy can include the simultaneous or near simultaneous administration of an anti-TNF therapy and an anti-IL-17 therapy. In other embodiments, a combination therapy can include the administration of an anti-TNF therapy followed by an anti-IL-17 therapy, where the separation in such that both the anti-TNF therapy and the anti-IL-17 therapy act concomitantly and/or achieve a synergistic effect. In other embodiments, a combination therapy can include the administration of an anti-IL-17 therapy followed by an anti-TNF therapy, where the separation in such that both the anti-TNF therapy and the anti-IL-17 therapy act concomitantly and/or achieve a synergistic effect. In an embodiment, the combination therapy includes both an anti-TNF therapy and an anti-IL-17 therapy in the same formulation (e.g., as a single molecule or as two separate molecules). In other embodiments, the combination therapy includes two separate formulations, one including an anti-TNF therapy and another including an anti-IL-17.

In one embodiment, the combination therapy can be a DVD-Ig binding protein (e.g., and anti-TNF-αnti-IL-17 DVD-Ig) as described in, for example, WO/2010/102251, incorporated herein by reference in its entirety.

In one embodiment, the combination therapy can be a DVD-Ig binding protein (e.g., and anti-TNF-αnti-IL-17 DVD-Ig) as described in, for example, WO/2010/102251, incorporated herein by reference in its entirety.

As used herein, the term “therapeutically effective amount” means an amount of an anti-TNF treatment and an anti-IL-17 treatment as described herein, which is capable of treating inflammatory disease (e.g., RA). The dose of a therapy to be administered according to this invention will, of course, be determined in light of the particular circumstances surrounding the case including, for example, the therapy administered, the route of administration, condition of the patient, and the pathological condition being treated, for example, the severity of the RA in the subject.

For administration to a subject, the combination therapy typically is formulated into a pharmaceutical composition comprising an anti-TNF treatment and an anti-IL-17 treatment and a pharmaceutically acceptable carrier. Therapeutic compositions typically should be sterile and adequately stable under the conditions of manufacture and storage.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral (e.g., intravenous, intramuscular, subcutaneous, intrathecal) administration (e.g., by injection or infusion). Depending on the route of administration, the active compound may be coated in a material to protect the compound from the action of acids and other natural conditions that may inactivate the compound.

There are numerous types of anti-inflammatory approaches that can be used in conjunction with the combination therapy comprising an anti-TNF treatment and an anti-IL-17 treatment, according to the invention. These include, for example, nonsteroidal anti-inflammatory drugs (NSAIDs), steroids, disease-modifying antirheumatic drugs (DMARDs) including methotrexate (Trexall), leflunomide (Arava), hydroxychloroquine (Plaquenil), sulfasalazine (Azulfidine) and minocycline (Dynacin, Minocin), and immunosuppressants including azathioprine (Imuran, Azasan), cyclosporine (Neoral, Sandimmune, Gengraf) and cyclophosphamide (Cytoxan).

The methods of the invention can employ these approaches to treat the same types of inflammatory disease as those for which they are known in the art to be used, as well as others, as can be determined by those of skill in this art. Also, these approaches can be carried out according to parameters (e.g., regimens and doses) that are similar to those that are known in the art for their use. However, as is understood in the art, it may be desirable to adjust some of these parameters, due to the additional use of an anti-TNF treatment and an anti-IL-17 treatment, with these approaches. For example, if another drug is normally administered as a sole therapeutic agent, when combined with an anti-TNF treatment and an anti-IL-17 treatment according to the invention, it may be desirable to decrease the dosage of the drug, as can be determined by those of skill in this art.

The present invention is further illustrated by the following examples which should not be construed as further limiting. The contents of all references, patents and published patent applications cited throughout this application, as well as the Figures are expressly incorporated herein by reference in their entirety.

### EXAMPLES

### Example 1

### Efficacy of Anti-TNFα/IL-17 DVD-Ig Protein in a Mouse Collagen Induced Arthritis Model

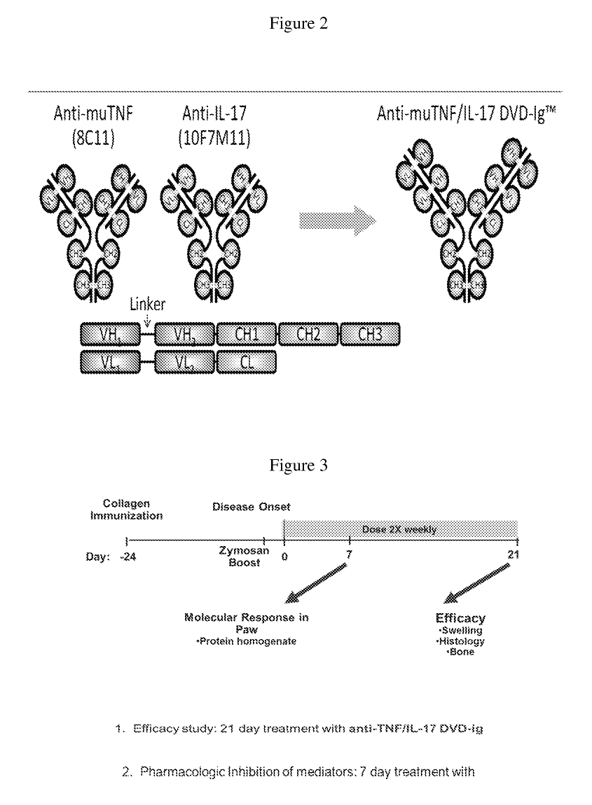

Anti-murine TNF antibody 8C11, anti-murine IL-17 antibody MAB421, both anti-TNF and anti-IL-17 antibodies, or an anti-mouse TNF/IL-17 DVD-Ig protein 8C11/10F7M11 (Tables 4 and 5) were tested in a mouse CIA model to determine whether dual neutralization of TNF and IL-17 with a bispecific molecule utilizing dual variable domain technology would confer efficacy in an arthritis model with the intended pharmacologic activity in the joint (FIG. 1, panels A-E). FIG. 2 shows a schematic of an anti-murine TNF/IL-17 DVD-Ig protein, composed of 8C11 (anti-murine TNF antibody), and 10F7M11 (anti-murine IL-17 antibody). The amino acid sequences of the variable domains and CDRs of the antibodies and DVD-Ig proteins used in these studies are provided below in Tables 1-4.

TABLE 1
Sequences of 8C11 and 10F7M11 Antibody Variable Domains

| SEQ ID | | Variable | |
|---|---|---|---|
| NO | Clone | Domain | 123456789012345678901234567890 |
| 1 | 8C11-VH | VH | EFQLQQSGPELVKPGASVRISCKASGYSFTDYN |
| | | | MNWVKQSNGKSLEWVGVINPNYGSSTYNQKFKG |
| | | | KATLTVDQSSSTAYMQLNSLTSEDSAVYYCARK |
| | | | WGQLGRGFFDVWGTGTTVTVSS |
| 2 | 8C11-VL | VL | QIVLSQSPAILSASPGEKVTMTCRASSSVSYMH |
| | | | WFQQKPGSSPKPWIYATSNLASGVPARFSGSGS |
| | | | GTSYSLTISRVEAEDAATYYCQQWSSSPLTFGA |
| | | | GTKLELKR |
| 3 | 10F7M11-VH | VH | QVQLQQSGAELVRPGTSVTLSCKASGYIFTDYE |
| | | | IHWVKQTPVHGLEWIGVNDPESGGTFYNQKFDG |
| | | | KAELTADKSSSTAYMELRSLTSEDSGVYYCTRY |
| | | | YRYESFYGMDYWGQGTSITVSS |
| 4 | 10F7M11-VL | VL | QIVLTQSPAIMSASPGEKVTMTCSASSSISYIY |
| | | | WFQQKPGTSPKRWIYATFELASGVPARFSGSGS |
| | | | GTSYSLTISSMEAEDAATYYCHQRSSYPWTFGG |
| | | | GSKLEIKR |

TABLE 2
Sequences of anti-TNFα Antibody 8C11 CDRs

| | |
|---|---|
| VH 8C11 CDR Set | |
| VH 8C11 CDR-H1 | Residues 31-35 of SEQ ID NO: 1 |
| VH 8C11 CDR-H2 | Residues 50-66 of SEQ ID NO: 1 |
| VH 8C11 CDR-H3 | Residues 99-109 of SEQ ID NO: 1 |
| VH 8C11 CDR Set | |
| VL 8C11 CDR-L1 | Residues 24-33 of SEQ ID NO: 2 |
| VL 8C11 CDR-L2 | Residues 49-55 of SEQ ID NO: 2 |
| VL 8C11 CDR-L3 | Residues 88-96 of SEQ ID NO: 2 |

TABLE 3
Sequences of Anti-IL-17 Antibody 10F7M11 CDRs

| | |
|---|---|
| VH 10F7M11 CDR Set | |
| VH 10F7M11 CDR-H1 | Residues 31-35 of SEQ ID NO: 3 |
| VH 10F7M11 CDR-H2 | Residues 50-66 of SEQ ID NO: 3 |
| VH 10F7M11 CDR-H3 | Residues 99-110 of SEQ ID NO: 3 |
| VH 10F7M11 CDR Set | |
| VL 10F7M11 CDR-L1 | Residues 24-33 of SEQ ID NO: 4 |
| VL 10F7M11 CDR-L2 | Residues 49-55 of SEQ ID NO: 4 |
| VL 10F7M11 CDR-L3 | Residues 88-96 of SEQ ID NO: 4 |

TABLE 4
Sequences of the Anti-mouse
TNF/IL-17 DVD-Ig Protein

| | | |
|---|---|---|
| DVD HEAVY | SEQ ID NO.: 5 | EFQLQQSGPELVKPGASVR |
| VARIABLE | | ISCKASGYSFTDYNMNWV |
| 8C11-linker- | | KQSNGKSLEWVGVINPNY |
| 10F7M11-DVD | | GSSTYNQKFKGKATLTVD |
| | | QSSSTAYMQLNSLTSEDSA |
| | | VYYCARKWGQLGRGFFD |
| | | VWGTGTTVTVSSGGGGSG |
| | | GGGSQVQLQQSGAELVRP |
| | | GTSVTLSCKASGYIFTDYEI |
| | | HWVKQTPVHGLEWIGVN |
| | | DPESGGTFYNQKFDGKAE |
| | | LTADKSSSTAYMELRSLTS |
| | | EDSGVYYCTRYYRYESFY |
| | | GMDYWGQGTSITVSS |
| 8C11 VH | SEQ ID NO.: 1 | EFQLQQSGPELVKPGASVR |
| | | ISCKASGYSFTDYNMNWV |
| | | KQSNGKSLEWVGVINPNY |
| | | GSSTYNQKFKGKATLTVD |
| | | QSSSTAYMQLNSLTSEDSA |
| | | VYYCARKWGQLGRGFFD |
| | | VWGTGTTVTVSS |
| linker | SEQ ID NO.: 6 | GGGGSGGGGS |
| 10F7M11 VH | SEQ ID NO.: 3 | QVQLQQSGAELVRPGTSV |
| | | TLSCKASGYIFTDYEIHWV |
| | | KQTPVHGLEWIGVNDPES |
| | | GGTFYNQKFDGKAELTAD |
| | | KSSSTAYMELRSLTSEDSG |
| | | VYYCTRYYRYESFYGMDY |
| | | WGQGTSITVSS |
| CH | SEQ ID NO.: 7 | ASTKGPSVFPLAPSSKSTSG |
| | | GTAALGCLVKDYFPEPVT |
| | | VSWNSGALTSGVHTFPAV |
| | | LQSSGLYSLSSVVTVPSSSL |
| | | GTQTYICNVNHKPSNTKV |
| | | DKKVEPKSCDKTHTCPPCP |
| | | APEAAGGPSVFLFPPKPKD |
| | | TLMISRTPEVTCVVVDVSH |
| | | EDPEVKFNWYVDGVEVH |
| | | NAKTKPREEQYNSTYRVV |
| | | SVLTVLHQDWLNGKEYKC |
| | | KVSNKALPAPIEKTISKAK |
| | | GQPREPQVYTLPPSREEMT |
| | | KNQVSLTCLVKGFYPSDIA |
| | | VEWESNGQPENNYKTTPP |
| | | VLDSDGSFFLYSKLTVDKS |
| | | RWQQGNVFSCSVMHEAL |
| | | HNHYTQKSLSLSPGK |
| DVD LIGHT | SEQ ID NO.: 8 | QIVLSQSPAILSASPGEKVT |
| VARIABLE | | MTCRASSSVSYMHWFQQ |
| 8C11-linker- | | KPGSSPKPWIYATSNLASG |
| 10F7M11-DVD | | VPARFSGSGSGTSYSLTISR |
| | | VEAEDAATYYCQQWSSSP |
| | | LTFGAGTKLELKRGGSGG |
| | | GGSGQIVLTQSPAIMSASP |
| | | GEKVTMTCSASSSISYIYW |
| | | FQQKPGTSPKRWIYATFEL |
| | | ASGVPARFSGSGSGTSYSL |
| | | TISSMEAEDAATYYCHQRS |
| | | SYPWTFGGGSKLEIKR |
| 8C11 VL | SEQ ID NO.: 2 | QIVLSQSPAILSASPGEKVT |
| | | MTCRASSSVSYMHWFQQ |
| | | KPGSSPKPWIYATSNLASG |
| | | VPARFSGSGSGTSYSLTISR |
| | | VEAEDAATYYCQQWSSSP |
| | | LTFGAGTKLELKR |
| linker | SEQ ID NO.: 9 | GGSGGGGSG |
| 10F7M11 VL | SEQ ID NO.: 4 | QIVLTQSPAIMSASPGEKV |
| | | TMTCSASSSISYIYWFQQK |
| | | PGTSPKRWIYATFELASGV |
| | | PARFSGSGSGTSYSLTISSM |
| | | EAEDAATYYCHQRSSYPW |
| | | TFGGGSKLEIKR |
| CL | SEQ ID NO.: 10 | TVAAPSVFIFPPSDEQLKSG |
| | | TASVVCLLNNFYPREAKV |
| | | QWKVDNALQSGNSQESVT |
| | | EQDSKDSTYSLSSTLTLSK |
| | | ADYEKHKVYACEVTHQGL |
| | | SSPVTKSFNRGEC |

TABLE 5
Source And Binding Information Regarding Anti-TNF Antibody,
Anti-IL-17 Antibody And Anti-TNF/IL-17 DVD-Ig Protein

| | | | | | | Dose (mg/kg) | |
|---|---|---|---|---|---|---|---|
| | | | | IC₅₀ (nM) | | 21 day | 7 day |
| | Clone name | Source | Isotype | IL-17 | TNF | CIA | CIA |
| Anti- | 8C11 | AbbVie | Mouse | | 2 | 12 | 6 |
| TNF | | | IgG2c | | | | |
| Anti- | MAB421 | R&D | Rat | 0.7 | | 12 | 6 |
| IL-17 | | | IgG2a | | | | |
| Anti- | 8C11/ | AbbVie | Mouse | 0.13 | 2 | 16 | 0.1- |
| TNF/ | 10F7Mll | | IgG2a | | | | 10 |
| IL-17 | | | | | | | |
| DVD-Ig | | | | | | | |
| protein | | | | | | | |

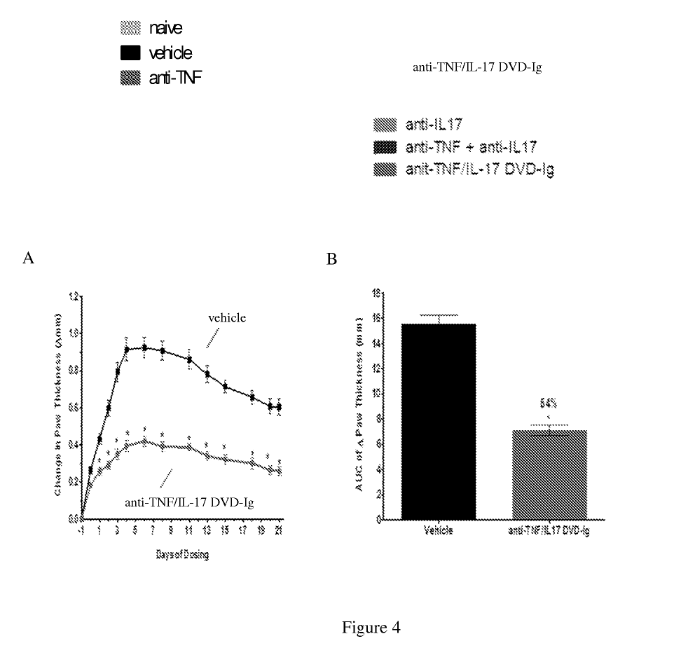

The efficacy of the anti-TNF/IL-17 DVD-Ig protein in the mouse CIA model of FIG. 3 are shown in FIG. 4, panels A and B. Male DBA/1J mice were injected intradermally (i.d.) at the base of the tail with 100 μL of an emulsion containing 100 μg of type II bovine collagen dissolved in 0.1N acetic acid and 100 μL of Complete Freund's Adjuvant containing 100 μg of Mycobacterium Tuberculosis H37Ra. Mice were boosted 21 days later intraperitoneally (i.p.) with 1.0 mg zymosan A in 200 μL of phosphate buffered saline (PBS). Disease onset occurred within 3 days of the boost. Mice were monitored for arthritis daily for the first week and monitored three times per week thereafter. The swelling of each paw was scored using a caliper Animals were treated twice per week (2×/week) with 16 mg/kg i.p. injection of the 8C11/10F7-DVD-Ig protein, which has specificity for mouse TNFα and IL-17. Mice receiving the anti-TNFα/IL-17 DVD-Ig protein had significantly reduced paw swelling over the 21 days of disease compared to animals receiving vehicle control (PBS).

### Example 2

### Anti-TNFα/IL-17 DVD-Ig Protein Inhibits Inflammation and Protects from Bone and Cartilage Loss

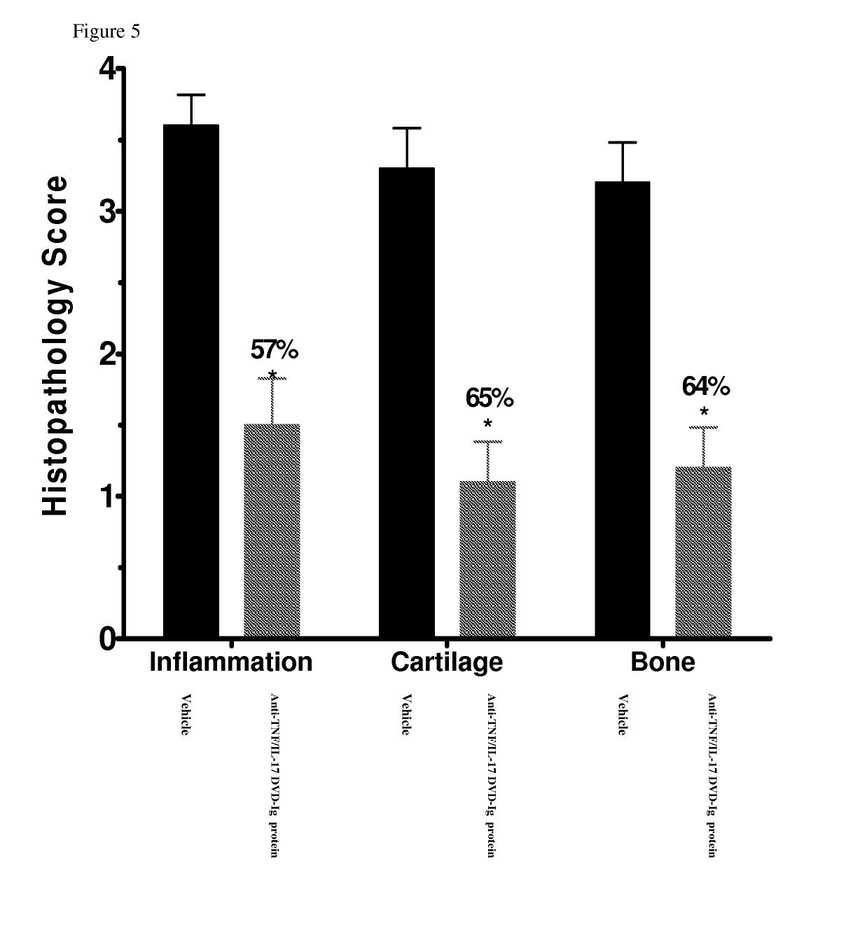

The efficacy of the anti-TNFα/IL-17 DVD-Ig protein was demonstrated by histologic changes to the arthritic joint (FIG. 5). Arthritis was induced in the DBA/1J mice as described in Example 1. Changes to the ultrastructure of the joint were evaluated at the termination of the study, three weeks after onset of arthritic signs. Formalin-fixed paws were sectioned and stained with Gills 3 hematoxylin (Richard-Allan Scientific) and eosin with phloxine (Newcomer Supply). The level of inflammation, cartilage and bone destruction in each sample was scored by a pathologist. Severity of disease was evaluated histologically using the following criteria: 0=normal; 1=minimal change; 2=mild change; 3=moderate change; and 4=severe change. Scores were summed for each animal and the total was expressed as an average of all animals in each group. Animals treated with anti-TNFα/IL-17 DVD-Ig protein demonstrated a significant reduction in inflammation, cartilage and bone destruction (FIG. 5).

### Example 3

### Comparison of Bone Protection by Anti-TNFα and Anti-IL-17, Alone and in Combination, in a Mouse CIA Model

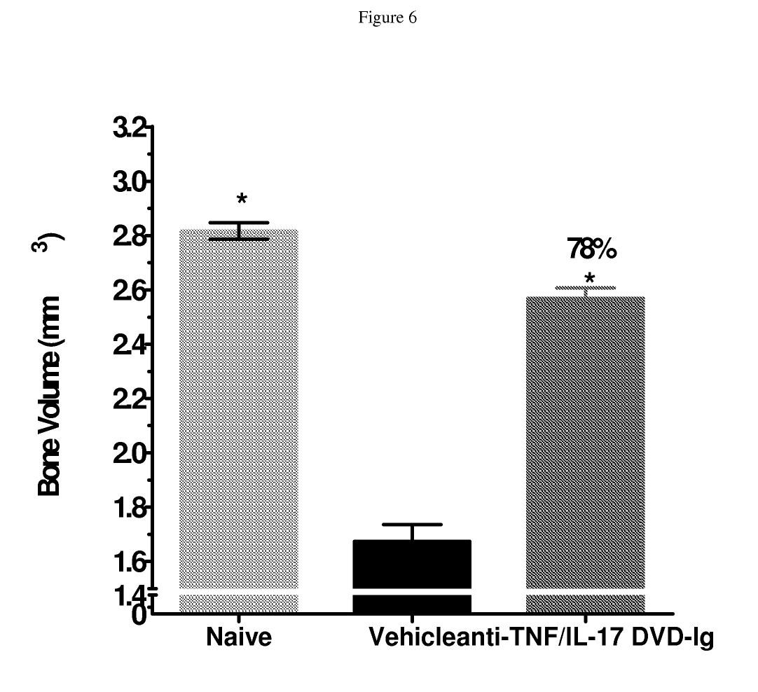

In this example, the efficacy of the combined blockade of TNF and IL-17 was demonstrated in a mouse CIA model with regard to protection from bone loss. Arthritis was induced in the DBA/1J mice as described in Example 1. The level of bone loss was evaluated at the termination of the study three weeks after the onset of arthritic signs. Hind paws were removed at the middle of the tibia/fibula and stored in 10% neutral buffered formalin. Paws were imaged using a Scanco μ CT40 (Scanco Medical AG) at 55 kVp and 145 μA, utilizing the High Resolution setting (1000 Projections/180° at 2048×2048 Pixel Reconstruction) and Isotropic Voxels with 180 millisecond integration time, resulting in a final isotropic voxel size of 18 μm×18 μm×18 μm. A cylindrical contour was manually drawn around a region of interest from the proximal junction of the calcaneous and navicular bone and extending into the tarsals for a fixed height of 100 slices (1.8 mm) In-house naïve controls have shown this region to give a highly conserved and statistically reproducible volumetric region for analysis. A 3-D quantitative evaluation was performed using ScancoAG analytical software. The evaluation included analysis for bone volume (mm³) and surface area to volumetric ratio, giving an approximation of tarsal surface roughness (mm⁻¹) Analytical settings of 0.8 sigma gauss and 1.0 were used, with an upper threshold of 1000 and a lower threshold of 320. There was a significant loss in bone volume in arthritic mice receiving vehicle treatment. In contrast, treatment with the anti-TNFα/IL-17 DVD-Ig protein resulted in significant protection from bone loss by 78%, respectively (p value <0.05 vs vehicle control)(FIG. 6).

### Example 4

### Anti-TNFα/IL-17 DVD-Ig Protein Inhibits TNFα Induced Mediators of Arthritis in the Joint of Mice with CIA

The pharmacologic activity of the TNFα binding domain of the anti-TNFα/IL-17 DVD-Ig protein in the joint of arthritic mice was demonstrated in a mouse CIA model. Arthritis was induced in DBA/1 mice as described in Example 1. Mice were treated with either anti-TNFα antibody 8C11 (6 mg/kg), anti-IL-17 antibody MAB421 (6 mg/kg), the combination of both anti-TNFα and anti-IL-17 Abs (both at 6 mg/kg), or the anti-TNFα/IL-17 DVD-Ig protein at 0.1 to 10 mg/kg 2x per week for 7 days. At the end of 7 days, the paws were collected from all animals and stored at −80° C. in liquid nitrogen. The frozen paws were pulverized using a Bio-Pulverizer (BioSpec Products, Inc.) and homogenized in RIPA buffer using a bullet blender. Tubes were spun for 10 minutes at 10,000 RPM and the supernatants transferred to the assay plates. Paw homogenates were analyzed with a Milliplex Map Mouse selected cytokine/chemokine magnetic panel bead system (Millipore) and the concentrations of all analytes were derived from Bio-Plex System fluorescence values (Biorad).

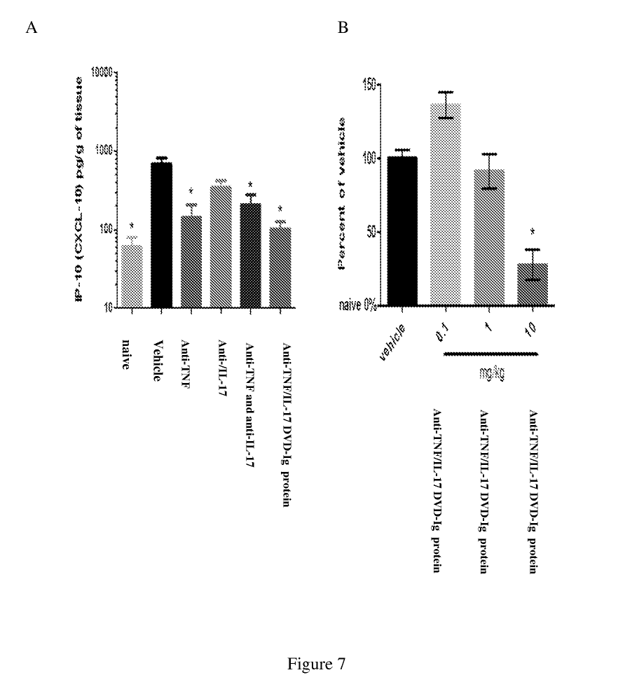

FIG. 7, panel A, shows that CXCL-10 protein, also known as IP-10, was up-regulated in arthritic paws 7 days after disease onset. This marker was significantly inhibited with treatment of the anti-TNFα Ab, but not by the anti-IL-17 Ab. Treatment with the anti-TNFα/IL-17-DVD-Ig protein effectively inhibited levels of CXCL-10 to levels comparable to that achieved with anti-TNFα treatment. This demonstrates the pharmacologic activity of the anti-TNFα/IL-17 DVD-Ig protein on the TNFα-driven induction of CXCL-10 protein. FIG. 7, panel B, shows that the anti-TNFα/IL-17 DVD-Ig protein dose-dependently inhibited CXCL-10 levels in the paw.

### Example 5

### Anti-TNFα/IL-17 DVD-Ig Protein Inhibits Mediators Cooperatively Regulated by TNFα and IL-17

The pharmacologic activity of the TNFα and IL-17 binding domains of the anti-TNFα/IL-17 DVD-Ig protein in the joint of arthritic mice was demonstrated in the mouse CIA model. Disease induction, treatment, and analysis methods were the same as those used in Example 4.

Briefly, male DBA/1J mice were injected i.d. at the base of the tail with 100 μL of emulsion containing 100 μg of Type II Bovine Collagen dissolved in 0.1N acetic acid and 100 μL of Complete Freund's Adjuvant containing 100 μg of Mycobacterium Tuberculois H37Ra. Mice were boosted 21 days later by i.p. injection with 1.0 mg Zymosan A in 200 μL PBS. Disease onset occurred within 3 days of the boost. Mice were monitored for arthritis daily for the first week and three times per week thereafter. Each paw was scored by a change in paw swelling as measured using a Caliper Thickness-Gage (Dyer, 310-115). Mice were enrolled into groups at the first clinical signs of disease with a maximal score of 2. Upon enrollment, mice were randomized into treatment cohorts consisting of monotherapy (6 mg/kg, either antibody), combination therapy (6 mg/kg each antibody) or anti-TNFα/IL-17 DVD-Ig protein (0.1 to 10 mg/kg) 2x per week for 7 days. Doses were selected based on previous dose-response experiments that determined 6 mg/kg to be the maximum effective dose. At the end of 7 days, the paws were collected from all animals and stored at −80° C. in liquid nitrogen. The frozen paws were pulverized with Bio-Pulverizer (BioSpec Products, Inc.) and homogenized in RIPA buffer using a bullet blender. Once homogenized, tubes were spun for 10 minutes at 10,000 RPM and the supernatants transferred to the assay plates. Paw homogenates were analyzed with a Milliplex Map Mouse selected cytokine/chemokine magnetic panel bead system (Millipore) and the concentrations for all analytes were derived from Bio-Plex System fluorescence values (Biorad).

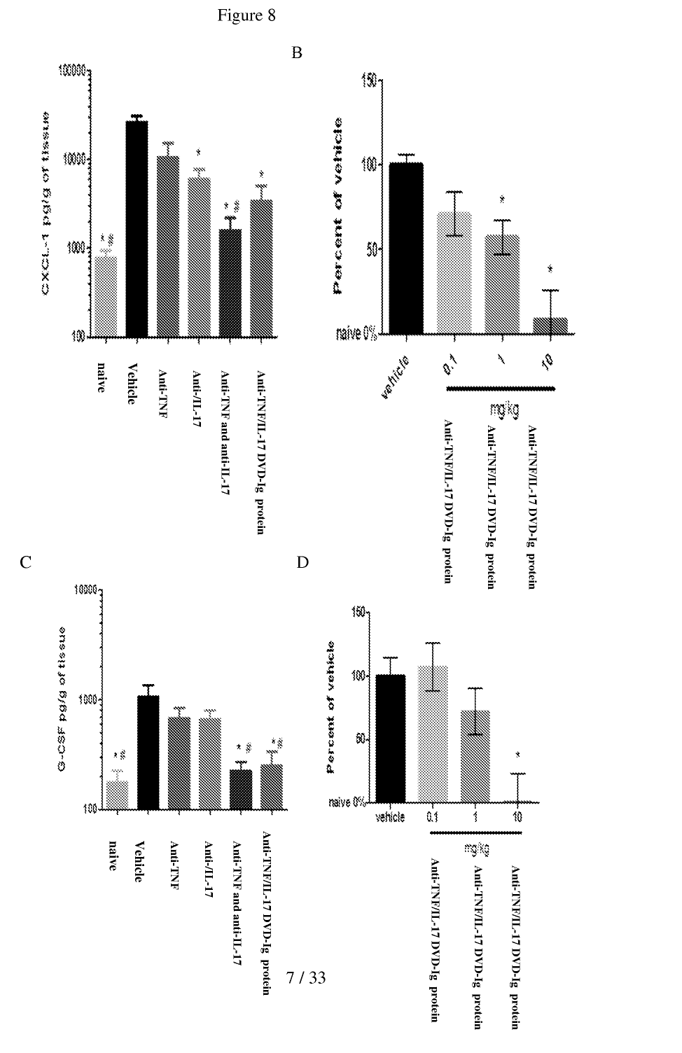

FIG. 8, panels A-D, show that CXCL-1 and G-CSF were both up-regulated in the arthritic paws 7 days after disease onset. CXCL-1 protein levels were not reduced by anti-TNFα treatment alone and modestly reduced by anti-IL-17 Ab treatment whereas a much greater reduction was observed with dual treatment with the anti-TNFα and anti-IL-17 antibodies in combination as well as with the anti-TNFα/IL-17 DVD-Ig protein. Similarly, G-CSF protein levels were up-regulated 7 days after the onset of CIA. This mediator was not inhibited with treatment with either anti-TNFα antibody or anti-IL-17 antibody alone but was significantly inhibited by dual treatment with the anti-TNFα and anti-IL-17 antibodies. A similar level of inhibition of G-CSF was observed with treatment of the anti-TNFα/IL-17 DVD-Ig protein demonstrating the pharmacologic activity of both the TNFα and IL-17 binding domains of the DVD-Ig in the joint of arthritic mice and that G-CSF provides a biomarker for IL-17 and TNF combination therapy.

### Example 6

### Anti-TNFα/IL-17 DVD-Ig Protein Inhibits Peripheral Blood Mononuclear Cell Production of GM-CSF and Decreases Lymphocyte Expression of CXCR4 in Healthy Subjects

TNF and IL-17 contribute to the pathogenesis of several inflammatory disorders and synergistically induce chemokines and cytokines, including chemokine (C-X-C motif) ligands 1 (CXCL1; GROa), 5 (CXCL5; ENA78), and 8 (CXCL8), chemokine (C-C motif) ligand 2 (CCL2; MCP-1), IL-1β, IL-6, G-CSF, and GM-CSF. See Griffin et al. (2012) J. Immunol. 188(12):6287-6299; Katz et al. (2001) Arthritis Rheum. 44(9):2176-2184; Laan et al. (2003) Eur. Respir. J. 21(3):387-393. In addition, the CXCL12 chemokine receptor, CXCR4, may also be regulated by TNF and IL-17. See Brembilla et al. (2013) Arthritis Res. Ther. 15(5):R151; Zrioual et al. (2009) J. Immunol. 182(5):3112-3120. As these factors play a role in the pathogenesis of several autoimmune diseases, greater clinical responses in patients may be possible with dual neutralization of TNF and IL-17.

Changes in certain chemokine receptors or ex vivo cytokine responses have been reported following anti-TNF therapy in human patients with rheumatoid arthritis. See Hot et al. (2012) Ann Rheum. Dis. 71(8):1393-1401; Eriksson et al. (2013) Scand. J. Rheumatol. 42(4):260-265. The biologic response to human anti-TNFα/IL-17 DVD-Ig protein ABT-122 in healthy human volunteers was analyzed to determine whether the response is based on the inhibition of TNF and/or IL-17. Table 6 provides the amino acid sequence of ABT-122.

TABLE 6
Amino Acid Sequence of ABT-122, an
Anti-TNF/IL-17 DVD-Ig Binding Protein

| | | |
|---|---|---|
| DVD HEAVY | SEQ ID | EVQLVESGGGLVQPGRSLRLSCAASGFTFD |
| VARIABLE | NO.: 21 | DYAMHWVRQAPGKGLEWVSAITWNSGHIDY |
| D2E7-GS10-B6-17 | | ADSVEGRFTISRDNAKNSLYLQMNSLRAED |
| DVD-Ig Protein\| | | TAVYYCAKVSYLSTASSLDYWGQGTLVTVS |
| | | SGGGGSGGGGSEVQLVQSGAEVKKPGSSVK |
| | | VSCKASGGSFGGYGIGWVRQAPGQGLEWMG |
| | | GITPFFGFADYAQKFQGRVTITADESTTTA |
| | | YMELSGLTSDDTAVYYCARDPNEFWNGYYS |
| | | THDFDSWGQGTTVTVSS |
| D2E7 VH | SEQ ID NO.: 22 | EVQLVESGGGLVQPGRSLRLSCAASGFTFD |
| | | DYAMHWVRQAPGKGLEWVSAITWNSGHIDY |
| | | ADSVEGRFTISRDNAKNSLYLQMNSLRAED |
| | | TAVYYCAKVSYLSTASSLDYWGQGTLVTVS |
| | | S |
| LINKER | SEQ ID NO.: 23 | GGGGSGGGGS |
| B6-17 VH | SEQ ID NO.: 24 | EVQLVQSGAEVKKPGSSVKVSCKASGGSFG |
| | | GYGIGWVRQAPGQGLEWMGGITPFFGFADY |
| | | AQKFQGRVTITADESTTTAYMELSGLTSDD |
| | | TAVYYCARDPNEFWNGYYSTHDFDSWGQGT |
| | | TVTVSS |
| CH | SEQ ID NO.: 25 | ASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPS |
| | | NTKVDKKVEPKSCDKTHTCPPCPAPELLGG |
| | | PSVFLFPPKPKDTLMISRTPEVTCVVVDVS |
| | | HEDPEVKFNWYVDGVEVHNAKTKPREEQYN |
| | | STYRVVSVLTVLHQDWLNGKEYKCKVSNKA |
| | | LPAPIEKTISKAKGQPREPQVYTLPPSRDE |
| | | LTKNQVSLTCLVKGFYPSDIAVEWESNGQP |
| | | ENNYKTTPPVLDSDGSFFLYSKLTVDKSRW |
| | | QQGNVFSCSVMHEALHNHYTQKSLSLSPGK |
| DVD LIGHT | SEQ ID NO.: 26 | DIQMTQSPSSLSASVGDRVTITCRASQGIR |
| VARIABLED2E7- | | NYLAWYQQKPGKAPKLLIYAASTLQSGVPS |
| GS10-B6-17 DVD-Ig | | RFSGSGSGTDFTLTISSLQPEDVATYYCQR |
| Protein\| | | YNRAPYTFGQGTKVEIKRGGSGGGGSGEIV |
| | | LTQSPDFQSVTPKEKVTITCRASQDIGSEL |
| | | HWYQQKPDQPPKLLIKYASHSTSGVPSRFS |
| | | GSGSGTDFTLTINGLEAEDAGTYYCHQTDS |
| | | LPYTFGPGTKVDIKR |
| D2E7 VL | SEQ ID NO.: 27 | DIQMTQSPSSLSASVGDRVTITCRASQGIR |
| | | NYLAWYQQKPGKAPKLLIYAASTLQSGVPS |
| | | RFSGSGSGTDFTLTISSLQPEDVATYYCQR |
| | | YNRAPYTFGQGTKVEIKR |
| LINKER | SEQ ID NO.: 28 | GGSGGGGSG |
| B6-17 VL | SEQ ID NO.: 29 | EIVLTQSPDFQSVTPKEKVTITCRASQDIG |
| | | SELHWYQQKPDQPPKLLIKYASHSTSGVPS |
| | | RFSGSGSGTDFTLTINGLEAEDAGTYYCHQ |
| | | TDSLPYTFGPGTKVDIKR |
| CL | SEQ ID NO.: 30 | TVAAPSVFIFPPSDEQLKSGTASVVCLLNN |
| | | FYPREAKVQWKVDNALQSGNSQESVTEQDS |
| | | KDSTYSLSSTLTLSKADYEKHKVYACEVTH |
| | | QGLSSPVTKSFNRGEC |

Biomarker discovery efforts were conducted in Phase I trials looking for serum protein/mRNA markers, chemokine/cytokine receptor changes or changes in ex vivo cytokine responses of PBMCs from ABT-122-treated healthy volunteers. Twenty-four healthy volunteers/subjects were administered a single subcutaneous dose of ABT-122 (1.5 mg/kg subcutaneously) in a Phase I trial to evaluate the bioavailability of a high concentration formulation compared to the current low concentration formulation of ABT-122 DVD-Ig binding protein in healthy subjects (Study M14-346). Peripheral blood mononucleated cells (PBMCs) were collected prior to ABT-122 administration at baseline and at days 7, 15, 36, and 57 post-dosing, and were cryopreserved. Thawed PBMCs were either analyzed directly by flow cytometry for chemokine receptors CXCR1, CXCR4, and CXCR5, or stimulated with LPS or anti-CD3+anti-CD28. Supernatants from the stimulated cultures were analyzed by multiplex analysis (MAPx, Millipore EMD) for LIF, IFNγ, TNF, IL-22, CXCL8, CXCL1, CXCL5, CCL2, IL-1β, IL-6, IL-10, G-CSF, and GM-CSF. Cytokine and chemokine levels were normalized to cell viability using a CellTiter-Glo assay (Promega Corp., Madison, Wis.) performed at the end of the culture period. The concentration of each chemokine or cytokine was divided by the relative luminescent units for each sample. Furthermore, a 2-tailed paired t-test was used to compare pre- and post-dose time points for each chemokine or cytokine.

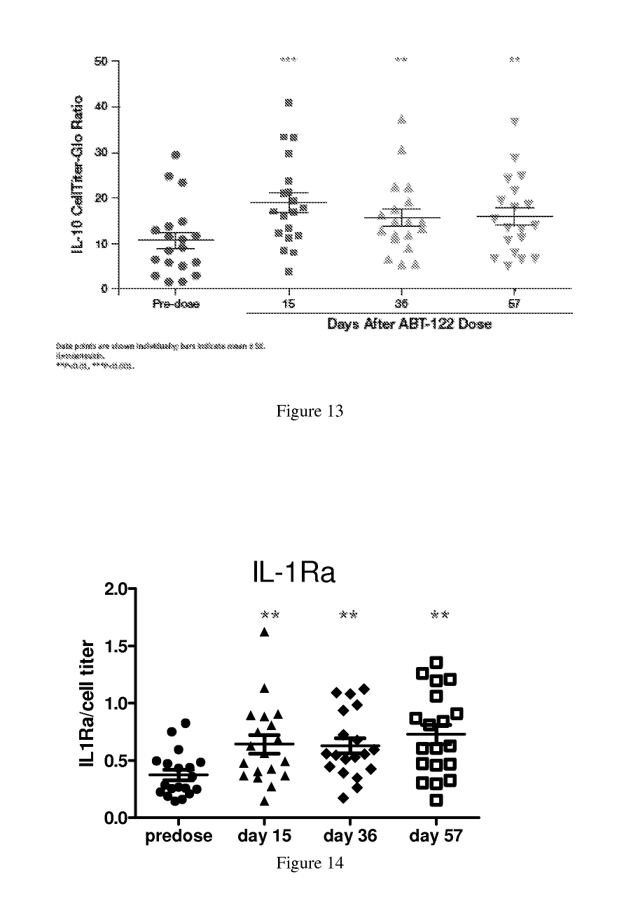

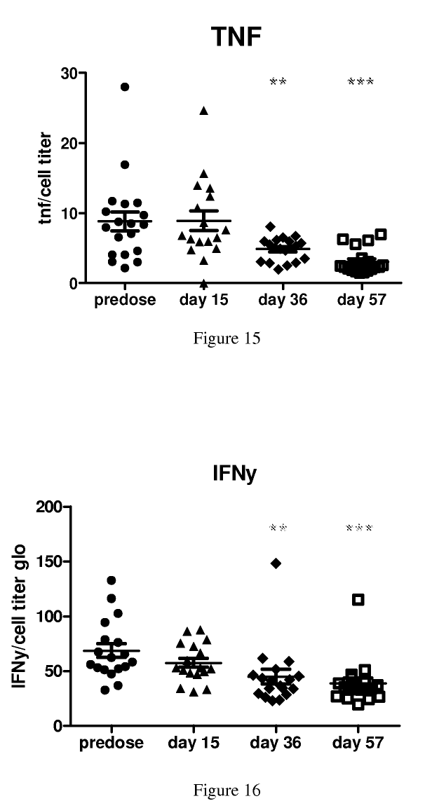

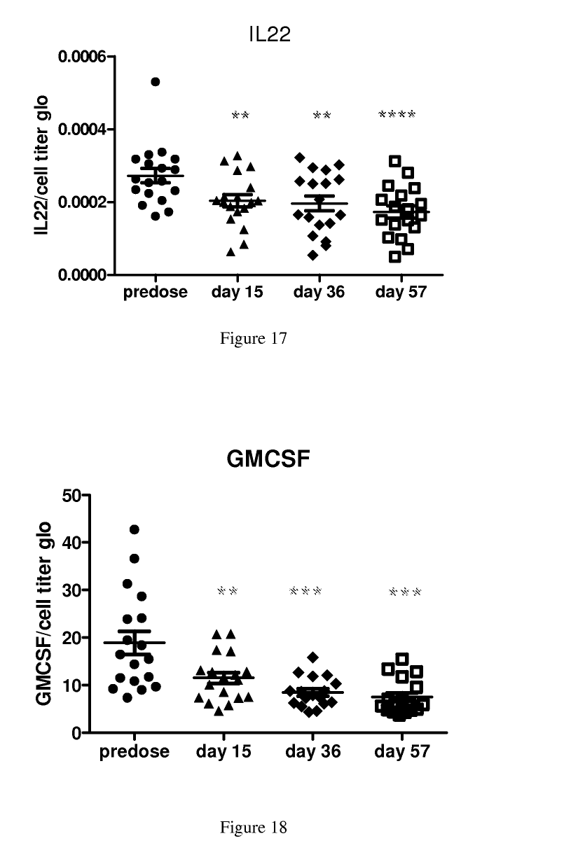

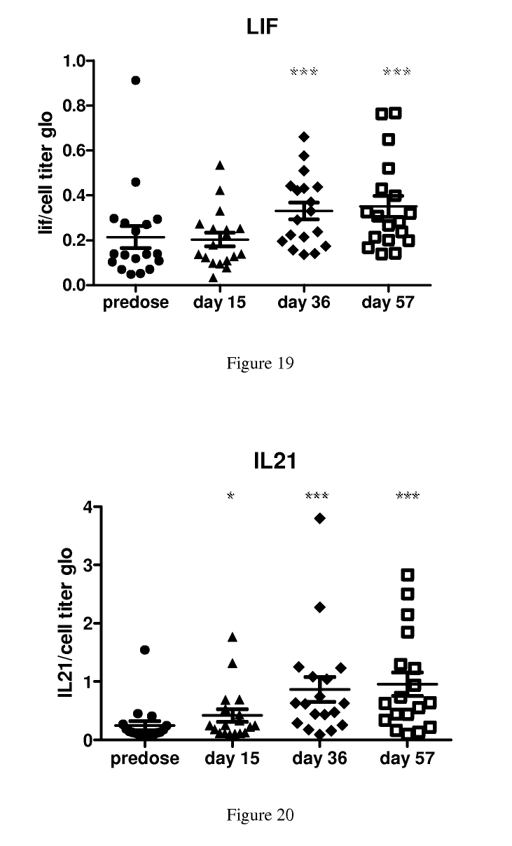

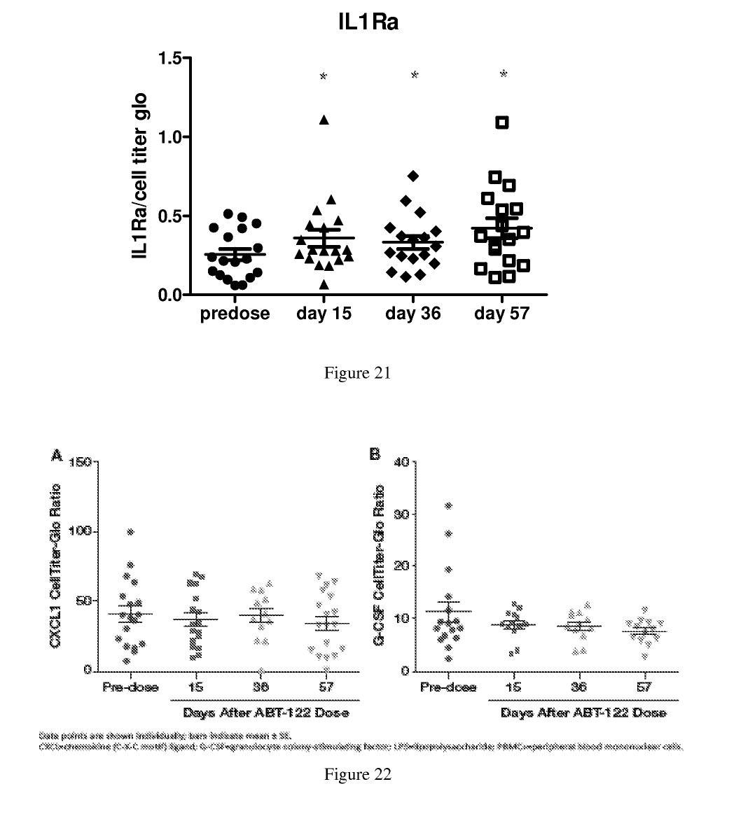

A single dose of ABT-122 administered to healthy volunteers resulted in significantly lower production of GM-CSF and TNF from day 15 through day 57 and day 36 through 57, respectively compared with baseline from LPS-stimulated PBMCs (FIGS. 12 and 15). In contrast, IL-1RA and IL-10 were increased in response to LPS stimulation of the PBMCs (FIGS. 13 and 14). Anti-CD3 plus anti-CD28 stimulation of the PBMCs resulted in lower production of IFNγ, GM-CSF and IL-22 compared to baseline from anti-CD3/CD28-stimulated PBMCs (FIGS. 16-18), whereas IL-21, IL-IRA and LIF were increased in these same cultures (FIGS. 19-21).

The decreased expression of GM-CSF is consistent with observations that TNF and IL-17 co-induce GM-CSF in mouse models and possibly reflect the dual neutralization activity of ABT-122. GM-CSF recruits neutrophils and monocytes/macrophages to sites of inflammation and thus reduction of this cytokine may contribute to a decrease in inflammation. Decreases in IFNγ, TNF and IL-22, which are all proinflammatory cytokines suggests additional, potentially novel, effects of ABT-122 on cellular immune responses. Increases in IL-1RA, an inhibitor of IL-1, and IL-10, which are immune regulatory molecules suggests that ABT-122 may promote immunoregulatory effects.

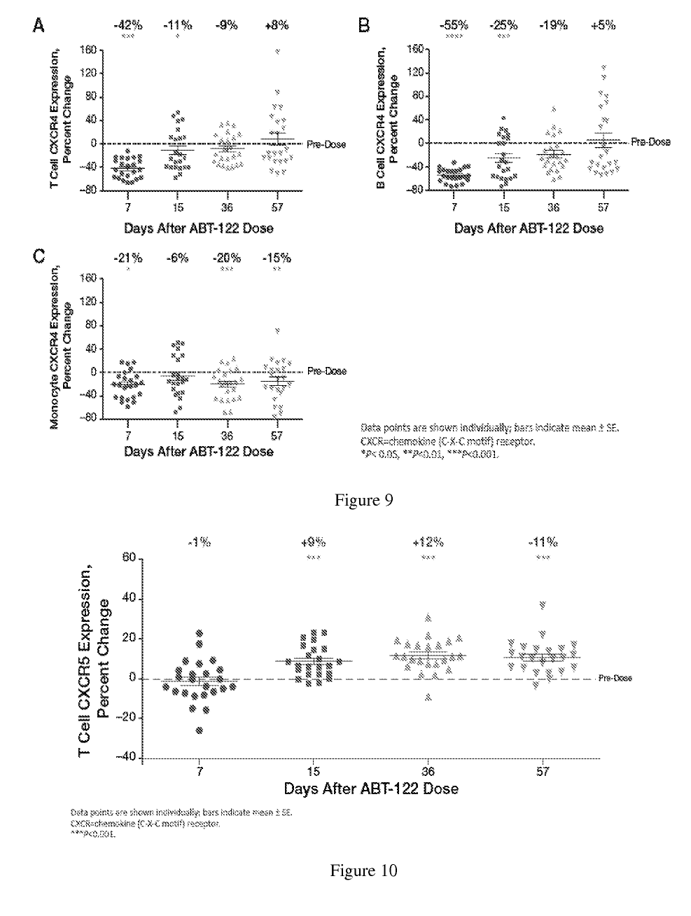

CXCR4 expression was decreased on B cells, T cells, and monocytes at day 7 post ABT-122 treatment compared with baseline with average reductions of 54%, 41%, and 20%, respectively. See FIG. 9, panels A-C. Decreases in CXCR4 expression on B cells persisted to day 15 (24%) and day 36 (18%). The early observation of decreased expression of CXCR4 is consistent with reported upregulation of CXCR4 expression on synoviocytes from patients with rheumatoid arthritis treated with IL-17 and TNF. Changes in CXCR4 have not been observed on T cells in patients with rheumatoid arthritis treated with anti-TNF antibody. See Eriksson et al. (2013) Scand. J. Rheumatol. 42(4):260-265. Modulation of CXCR4 expression may reflect the effects of dual neutralization by ABT-122. Expression of CXCR4 later returned to pre-dose levels, consistent with a single dose of ABT-122 binding protein. CXCR4 and/or its ligand CXCL12 (SDF-1a) are elevated in rheumatoid arthritis patients. These molecules may promote pathogenesis by recruiting activated T cells to the synovium. Thus decreases in CXCR4 expression may prevent recruitment of T cells. Interestingly, modest and more persistent decreases in CXCR4 expression were observed in monocytes, which may indicate differential responses of cell types to TNF and IL-17 neutralization. IL-17 and TNF were neutralized by ABT-122, accordingly data may indicate that GM-CSF and CXCR4 are synergistically regulated by IL-17 and TNF.

There were 2.5-fold elevations in the anti-inflammatory cytokine IL-10 (FIG. 13), and significant 9-12% increases in CXCR5 expression on T cells following administration of ABT-122 (FIG. 10). The calculated increases in CXCR5 expression are consistent with observations of increased CXCR5 expression on T cells in patients with rheumatoid arthritis who were treated with anti-TNF. These results may reflect the presence and effective treatment of the anti-TNF component of ABT-122. IL-10-producing T cells were increased in patients receiving anti-TNF treatment only, and thus, the observed increase in IL-10 is consistent with the anti-TNF effect on the ex vivo production of this cytokine. See Evans et al. (2014) Nat. Commun 5:3199. Since IL-10 is an immunoregulator, this cytokine may play a crucial role in the mechanism of action of anti-TNF antibodies in effectively treating rheumatoid arthritis.

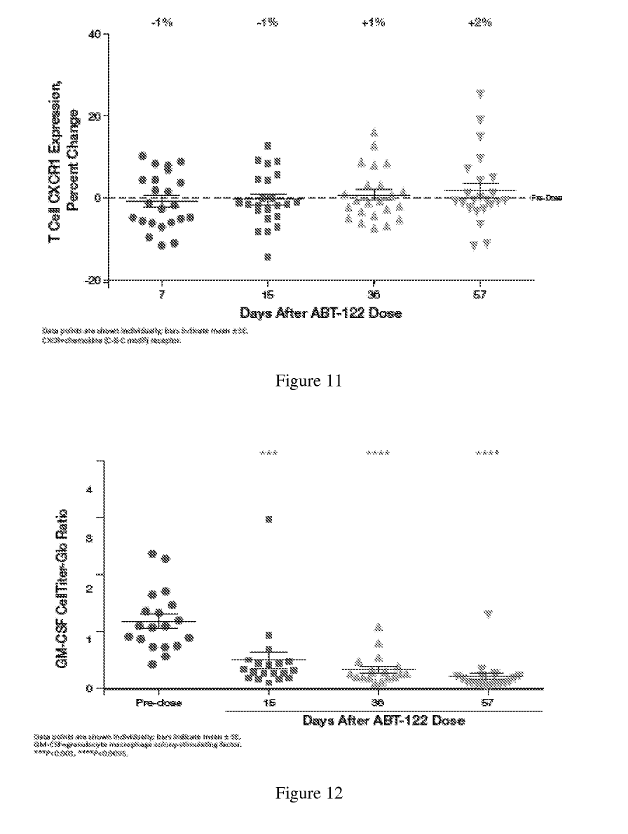

Expression of CXCR1 appeared to be unchanged after administration of ABT-122 (FIG. 11). These expression data are consistent with observations that decreased CXCR1 on T cells did not occur with short-term (2 weeks) anti-TNF treatment in patients with rheumatoid arthritis (Eriksson et al. (2013) Scand. J. Rheumatol. 42(4):260-265), although a significant reduction was reported with long-term (30 weeks) treatment. Expression levels of CXCL1 (FIG. 22, panel A) or G-CSF (FIG. 22, panel B) did not change in response to ex vivo LPS stimulation of PBMCs. No changes in IL-1β, IL-6, or CXCL8 were observed in response to LPS stimulation. IL-1β, IL-6, and/or CXCL8 are likely co-regulated by TNF and IL-17. See Griffin et al. (2012) J. Immunol. 188(12):6287-6299; Katz et al. (2001) Arthritis Rheum. 44(9): 2176-2184. CXCL5 and CCL2 were also not stimulated by LPS.

The data herein show novel changes in cellular responses mediated in vivo by ABT-122. It is possible that the effects on these cytokines/chemokines require the presence of TNF, IL-17, or ABT-122 in the cultures.

In summary, the changes observed in expression of GM-CSF and CXCR4 in healthy subjects, after dual neutralization of TNF and IL-17 by ABT-122, demonstrate the effective pharmacodynamic activity of ABT-122 DVD-Ig protein consistent with the combinatorial activities of TNF and IL-17 described in previous examples herein. The effects of ABT-122 on these analytes were demonstrated in healthy volunteers and thus are likely to reflect modulation of the in vivo homeostatic activities of TNF and IL-17 in the absence of disease. However, these data further support the rationale that ABT-122 can be used to evaluate the therapeutic potential of dual IL-17 and TNF blockade in patients with disorders driven by these two cytokines.

### Example 7

### ABT-122 Treatment of RA Patients Decreases CXCL9, CXCL10, CCL23 and Soluble e-Selectin Serum Levels

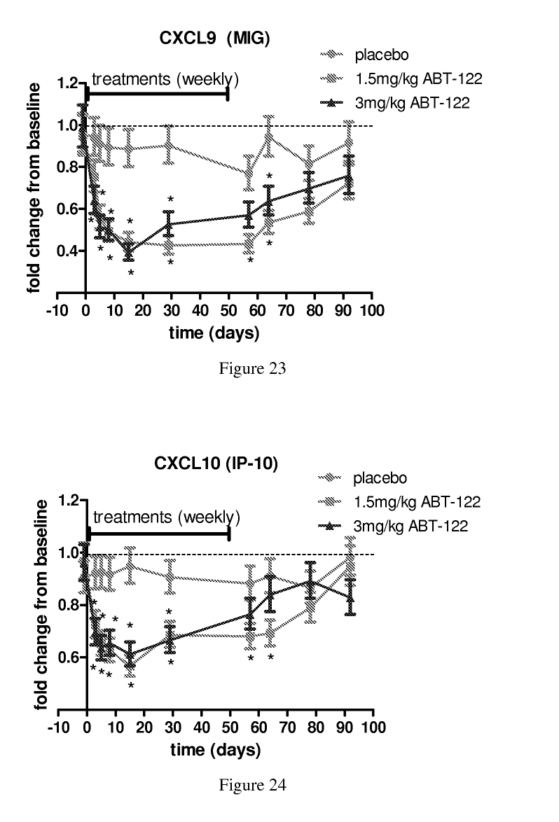

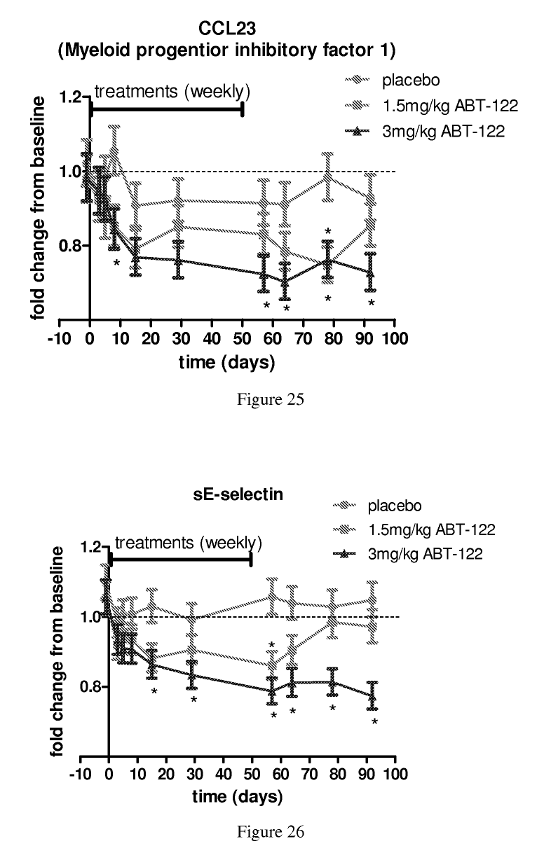

Clinical trial studies M14-048 and M12-962 involved a multiple ascending dose, double-blind, randomized study with stable RA subjects receiving stable doses of methotrexate (7.5-25 mg/wk) to assess the safety, tolerability, PK and exploratory pharmacodynamics of ABT-122. Subjects were subcutaneously administered either one of 4 dose regimens of ABT-122, 1 mg/kg every other week (4 doses), or 0.5, 1.5, or 3 mg/kg weekly (8 doses); or placebo and evaluated through 45 days following last dose. Serum samples for a panel of inflammation markers and chemokines based on preclinical studies with dual TNF and IL-17 neutralization, were collected at baseline through day 92 and analyzed by multiplex assays. For CXCL9 and CXCL10, rapid decreases relative to placebo occurred within 3 days of ABT-122 administration (−25-35%, and −30% from baseline for CXCL9 and CXCL10, respectively) (FIGS. 23 and 24). Maximal decreases occurred by day 15 (−60% and −45% for CXCL9 and CXCL10, respectively) and persisted through 14 days after last dose. Serum CCL23 also decreased following ABT-122 with maximal decreases (−30%) at day 64 and continued through day 92 (FIG. 25). Consistent with anti-TNF inhibition, soluble E-selectin levels decreased following ABT-122, persisting through day 92 for the 3.0 mg/kg group (FIG. 26). As CXCL9, CXCL10 and CCL23 are involved in lymphocyte and myeloid cell recruitment into inflamed tissues, decreases in these chemokines indicate that ABT-122 rapidly modulates potential pathophysiologic pathways in RA patients, with evidence for persistent effects after cessation of treatment.

### Example 8

### Evaluation of Cytokine and Chemokine Responses Mediated by ABBV-257, an Second Anti-TNF/IL-17 DVD-Ig Binding Protein

Clinical trial study M14-355 involved a single ascending dose, double-blind, randomized study with healthy adult subjects to assess the safety, tolerability, and PK of the ABBV-257, another anti-human TNF/IL-17 DVD-Ig binding protein, using a single dose IV infusion or a single dose SC injection of ABBV-257. Secondary objectives were to measure the ADA levels following the single IV or SC dose. An exploratory objective was to determine any change in biomarker assessments at multiple time points following study drug administration. The doses administered were 0.3 mg/kg (Group 1), 1.0 mg/kg (Group 2), and 3.0 mg/kg (Group 3) given IV and 0.3 mg/kg (Group 4) and 3 mg/kg (Group 4a) given SC. Eighteen subjects received IV doses and 12 subjects received SC doses of ABBV-257. Ten subjects received placebo control (6 in the IV administration arm and 4 in the SC administration arm). Table 7 provides the amino acid sequences for ABBV-257.

TABLE 7
Amino Acid Sequence of ABBV-257,
an Anti-TNF/IL-17 DVD-Ig Binding Protein

| | | |
|---|---|---|
| DVD HEAVY | SEQ ID NO.: 11 | EVQLVQSGAEVKKPGASVKV |
| VARIABLE | | SCKASGYTFANYGIIWVRQA |
| HMAK199-1- | | PGQGLEWMGWINTYTGKPTY |
| GS10-H10F7-M11 | | AQKFQGRVTMTTDTSTSTAY |
| DVD | | MELSSLRSEDTAVYYCARKL |
| | | FTTMDVTDNAMDYWGQGTTV |
| | | TVSSGGGGSGGGGSEVQLVQ |
| | | SGAEVKKPGSSVKVSCKASG |
| | | YTFTDYEIHWVRQAPGQGLE |
| | | WMGVNDPESGGTFYNQKFDG |
| | | RVTLTADESTSTAYMELSSL |
| | | RSEDTAVYYCTRYSKWDSFD |
| | | GMDYWGQGTTVTVSS |
| HMAK199-1VH | SEQ ID NO.: 12 | EVQLVQSGAEVKKPGASVKV |
| | | SCKASGYTFANYGIIWVRQA |
| | | PGQGLEWMGWINTYTGKPTY |
| | | AQKFQGRVTMTTDTSTSTAY |
| | | MELSSLRSEDTAVYYCARKL |
| | | FTTMDVTDNAMDYWGQGTTV |
| | | TVSS |
| LINKER | SEQ ID NO.: 13 | GGGGSGGGGS |
| H10F7-M11 VH | SEQ ID NO.: 14 | EVQLVQSGAEVKKPGSSVKV |
| | | SCKASGYTFTDYEIHWVRQA |
| | | PGQGLEWMGVNDPESGGTFY |
| | | NQKFDGRVTLTADESTSTAY |
| | | MELSSLRSEDTAVYYCTRYS |
| | | KWDSFDGMDYWGQGTTVTVS |
| | | S |
| CH CG1234, 235 | SEQ ID NO.: 15 | ASTKGPSVFPLAPSSKSTSG |
| MUT Z NONA | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| DVD LIGHT | SEQ ID NO.: 16 | DIQMTQSPSSLSASVGDRVT |
| VARIABLE | | ITCRASQDISQYLNWYQQKP |
| HMAK199-1- | | GKAPKLLIYYTSRLQSGVPS |
| GS10-H10F7- | | RFSGSGSGTDFTLTISSLQP |
| M11DVD | | EDFATYFCQQGNTWPPTFGQ |
| | | GTKLEIKRGGSGGGGSGDIQ |
| | | MTQSPSSLSASVGDRVTITC |
| | | RASSGIISYIDWFQQKPGKA |
| | | PKRLIYATFDLASGVPSRFS |
| | | GSGSGTDYTLTISSLQPEDF |
| | | ATYYCRQVGSYPETFGQGTK |
| | | LEIKR |
| HMAK199-1 | SEQ ID NO.: 17 | DIQMTQSPSSLSASVGDRVT |
| VL | | ITCRASQDISQYLNWYQQKP |
| | | GKAPKLLIYYTSRLQSGVPS |
| | | RFSGSGSGTDFTLTISSLQP |
| | | EDFATYFCQQGNTWPPTFGQ |
| | | GTKLEIKR |
| LINKER | SEQ ID NO.: 18 | GGSGGGGSG |
| H10F7-M11VL | SEQ ID NO.: 19 | DIQMTQSPSSLSASVGDRVT |
| | | ITCRASSGIISYIDWFQQKP |
| | | GKAPKRLIYATFDLASGVPS |
| | | RFSGSGSGTDYTLTISSLQP |
| | | EDFATYYCRQVGSYPETFGQ |
| | | GTKLEIKR |
| CL | SEQ ID NO.: 20 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

*Note that the component CDRS of the VH and VL binders are in bold

### Evaluation of Cell Surface Markers

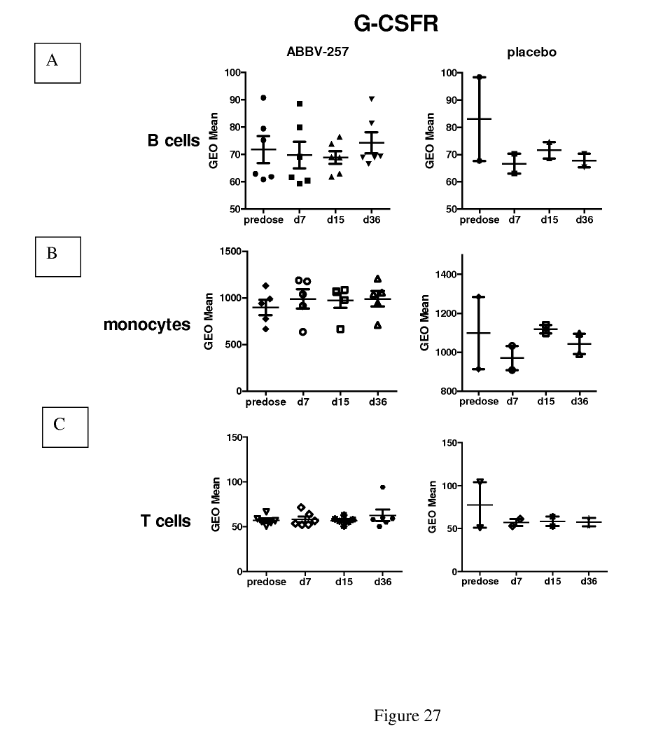

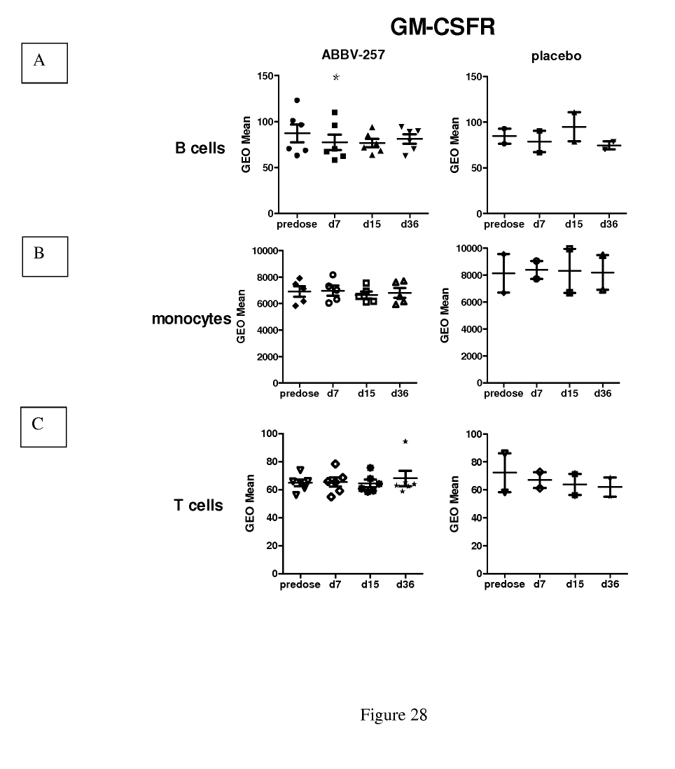

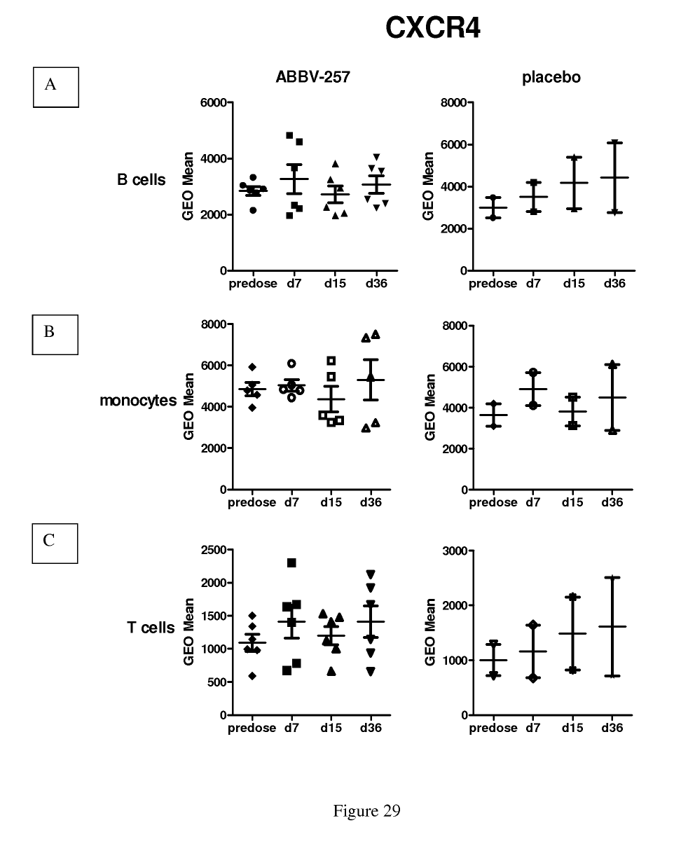

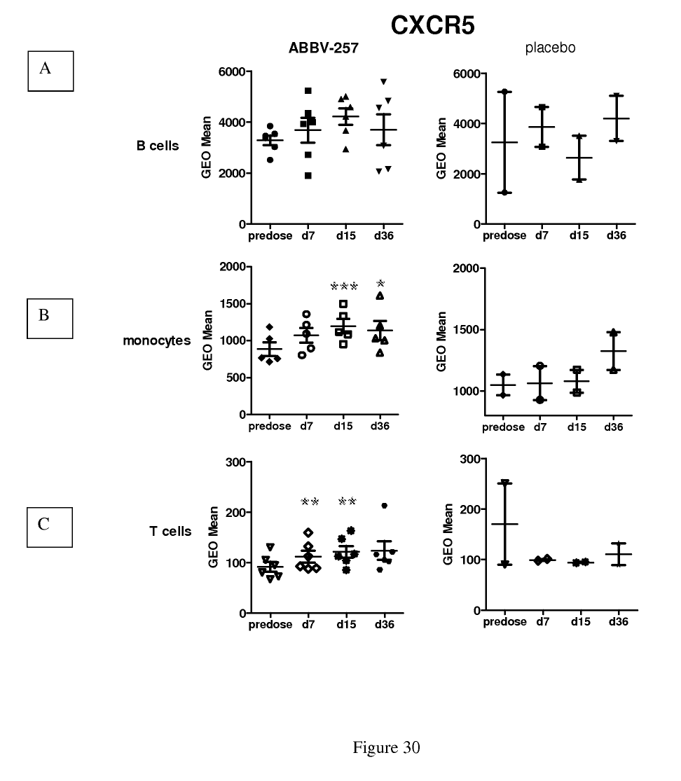

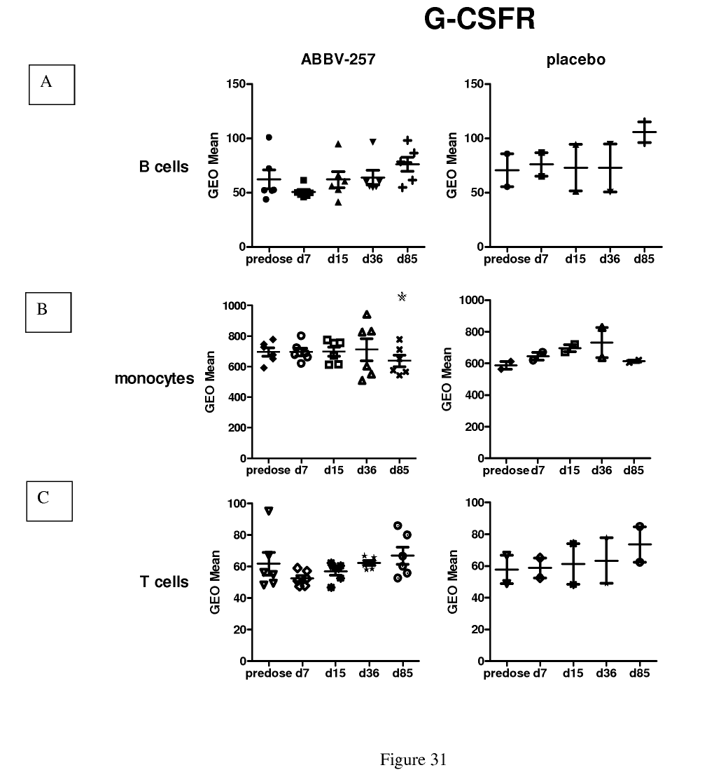

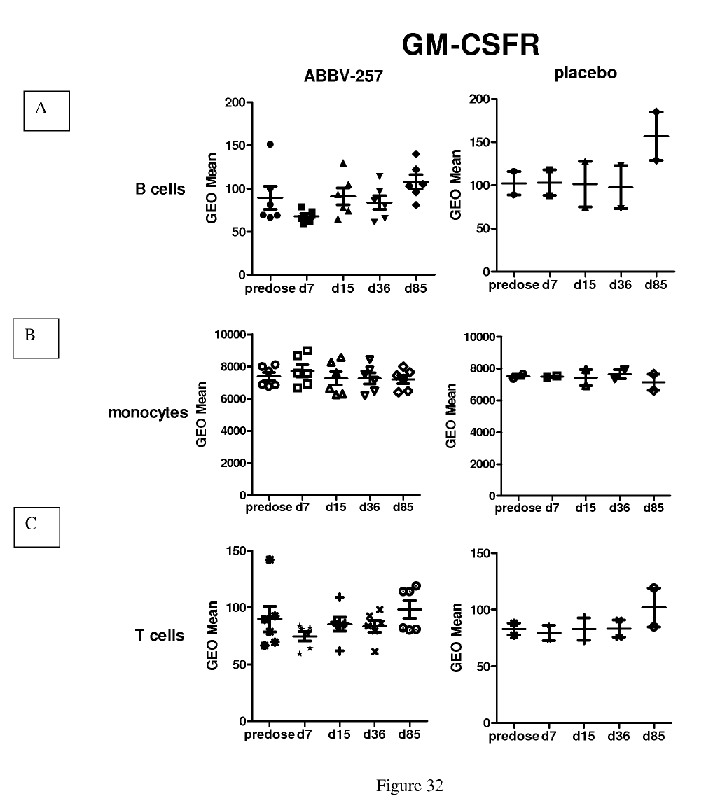

Biomarker analysis included analyzing PBMCs from study M14-346, as well as PBMCs collected from the 3.0 mg/kg IV and SC dose groups for ABBV-257 study M14-355. See Table 8 and FIGS. 27-34 (panels A-C for each).

For study M14-355, healthy volunteer subjects were intravenously or subcutaneously administered a single dose (3 mg/kg) of ABBV-257. PBMC samples were collected from the subjects on various days later (i.e., 1, 7, 15, 36 and 85 days after the single dose). Control PBMC samples were collected prior to the ABBV-257 being administered. Control subjects received a placebo. The samples were cryopreserved, thawed, and washed. The washed samples underwent flow cytometry analysis. Cytokine and chemokine receptors on T cells, B cells and monocytes were analyzed.

TABLE 8
Summary Of Study M14-355 (ABBV-257
Treatment) Cell Surface Marker Data

| | CXCR4 | CXCR5 | G-CSFR | GM-CSFR |
|---|---|---|---|---|
| T cells | — | ↑↑ | — | — |
| B cells | ↓ | ↑ | — | — |
| Monocytes | ↓ | ↑ | — | — |

TABLE 9
Summary Of Study M14-346 (ABT-122
Treatment) Cell Surface Marker Data

| | CXCR4 | CXCR5 | G-CSFR |
|---|---|---|---|
| T cells | ↓↓ | ↑ | — |
| B cells | ↓↓↓ | ↑↑ | ↓↓ |
| Monocytes | ↓ | — | ↓ |

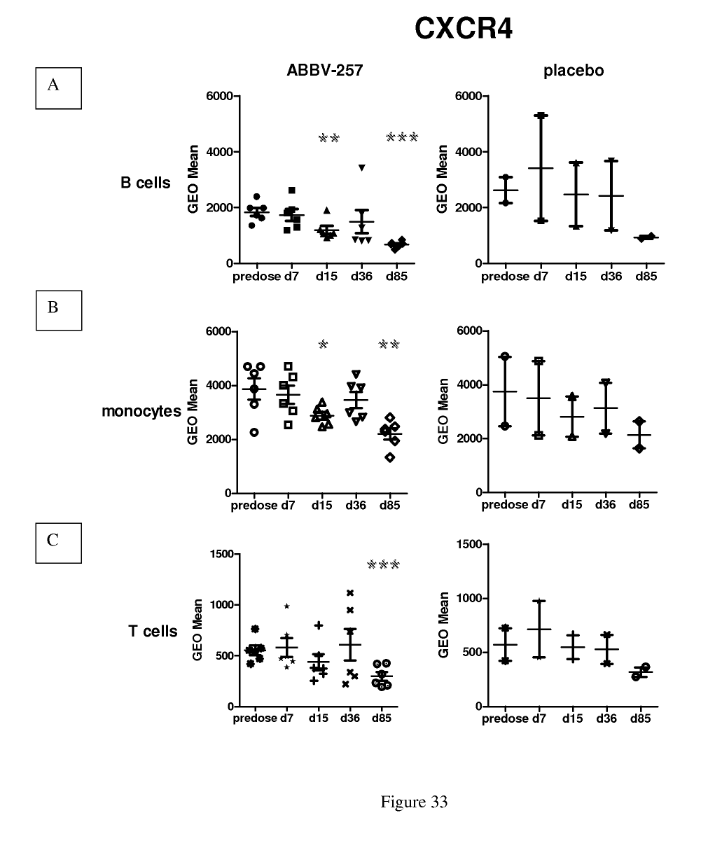

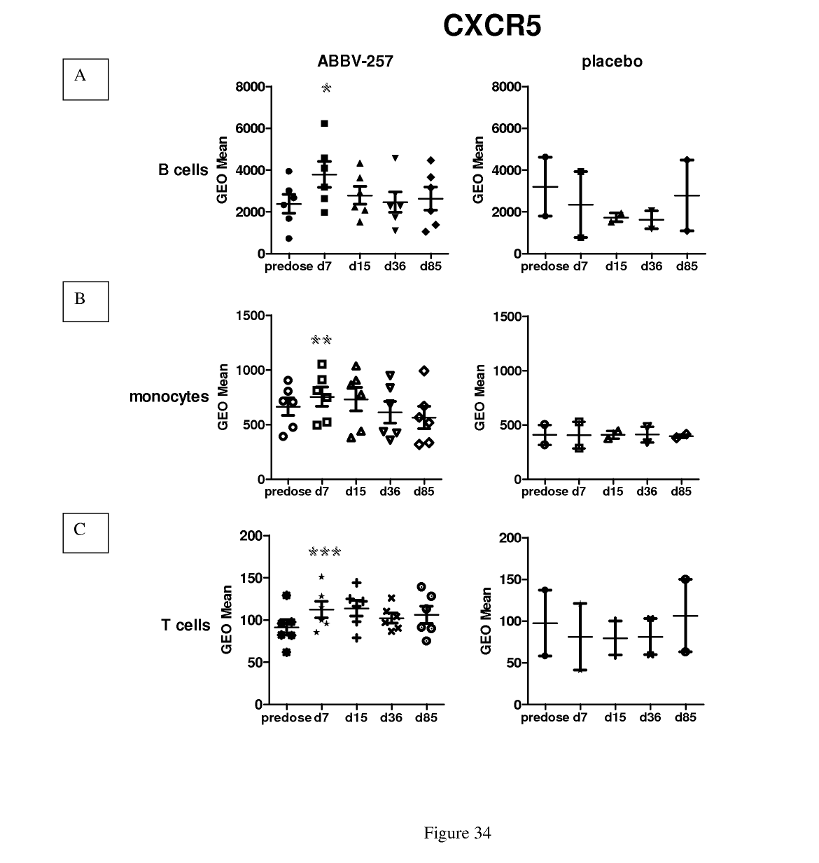

Similar results were observed for ABT-122 (Table 9) as compared to ABBV-257 (Table 8) for CXCR4 and CXCR5. See also ABBV-257 data for CXCR5 in IV group in FIG. 18, panel C and in SC group in FIG. 34, panels A and C, and CXCR4 in SC group in FIG. 33, panels A and B).

Thus, ABT-122 and ABBV-257 DVD-Ig binding proteins show a similar response with regard to cell surface markers.

### Ex Vivo Cytokine Responses

Ex-vivo cytokine responses for PBMCs from subjects administered ABBV-257 were also analyzed. Healthy volunteer subjects were intravenously or subcutaneously administered a single dose (3 mg/kg) of ABBV-257. PBMC samples were collected from the subjects several days later (i.e., 1, 7, 15, 36 and 85 days after the single dose). Control PBMC samples were collected prior to ABBV-257 administration. Control subjects received a placebo. The samples were cryopreserved, washed, and thawed. The washed samples were then stimulated with LPS, or CD3 and CD28. The samples were incubated for 24 hours or 48 hours. Multiplex cytokine/chemokine analysis was performed on the supernatant. Cell-titer Glo viability assays were performed for normalization of values.

For comparison, PBMCs from study M14-346 (single dose of ABT-122 at 1.5 mg/kg to healthy subjects) were also tested and are shown below in Table 10.

TABLE 10
Summary Of Biomarker Data for Cells from M14-346 (ABT-
122 Treatment) Treated With LPS Or Anti-CD3 And Anti-CD28

| Cytokines | LPS stimulation | Anti-CD3/28 stimulation |
|---|---|---|
| IL-1Ra | ↑ all timepoints | ↑ all timepoints |
| GM-CSF | ↓ all timepoints | ↓ all timepoints |
| TNF | ↓ late timepoints | ND |
| IL-10 | ↑ all timepoints | — |
| IFNγ | ND | ↓ late timepoints |
| IL-21 | ND | ↑ all timepoints |
| LIF | ND | ↑ late timepoints |

ND = not done

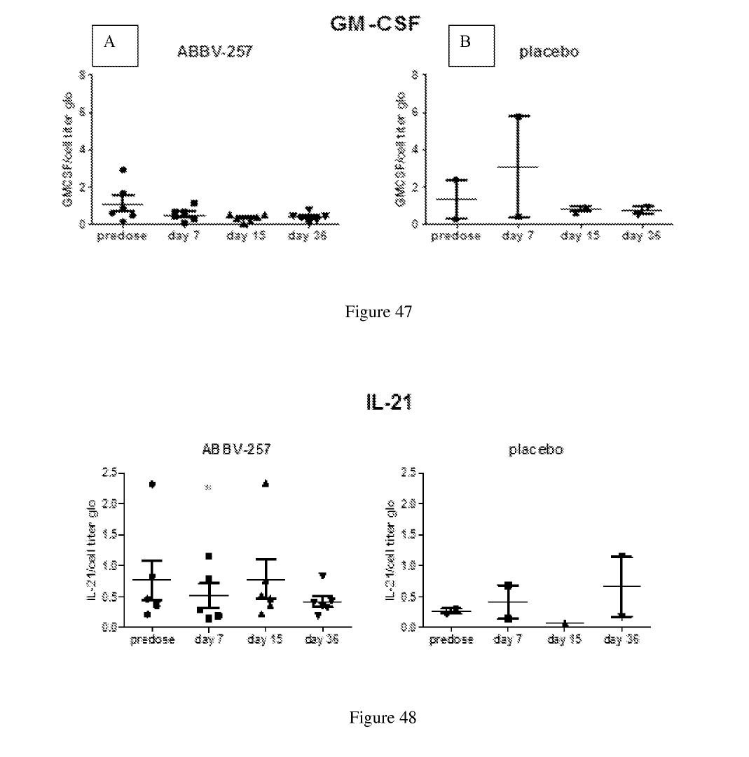

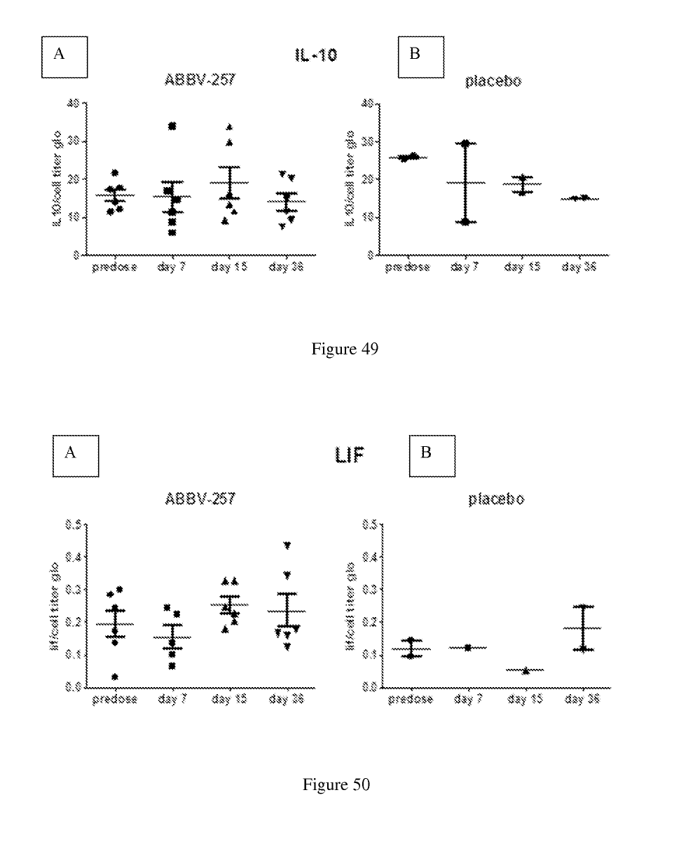

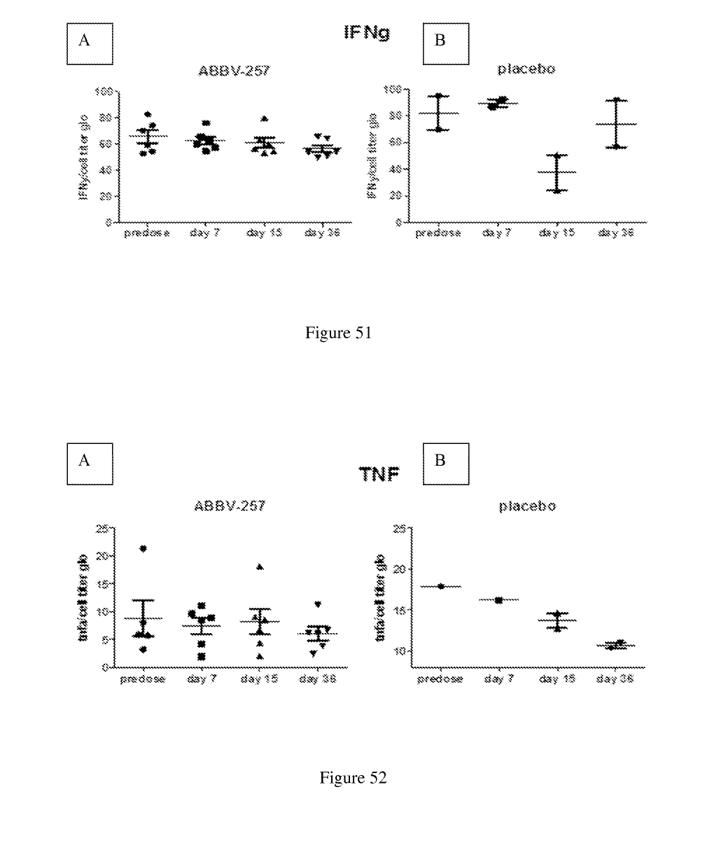

Minimal significant differences in cytokine responses in the IV dosing group were observed for subjects administered ABBV-257 DVD-Ig binding protein (FIGS. 46-52).

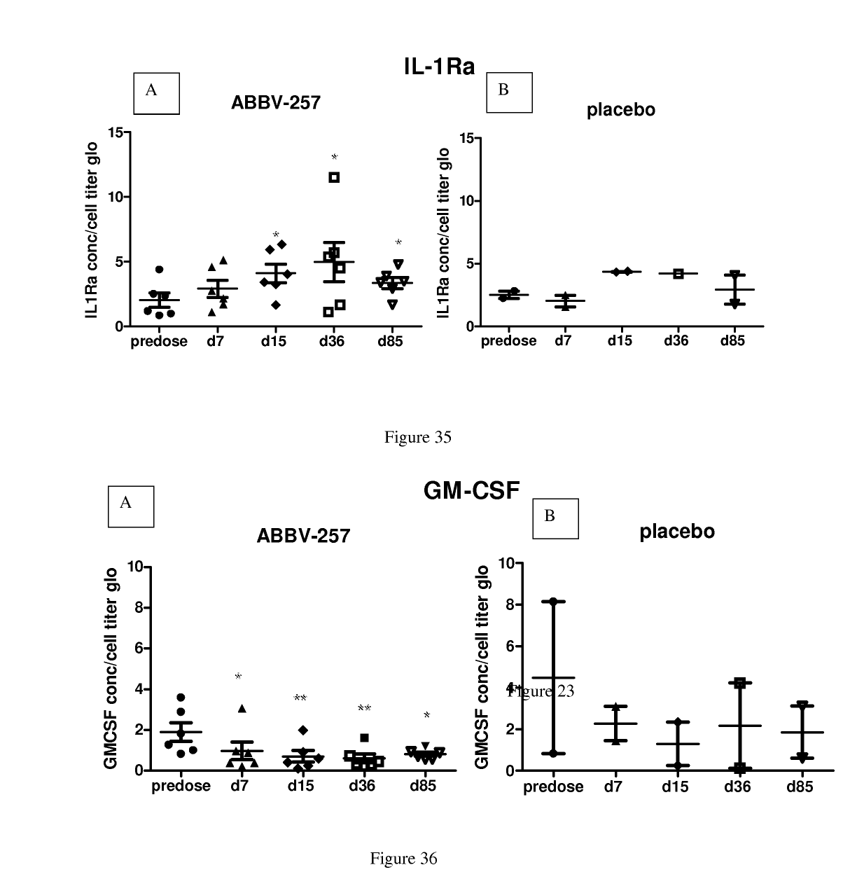

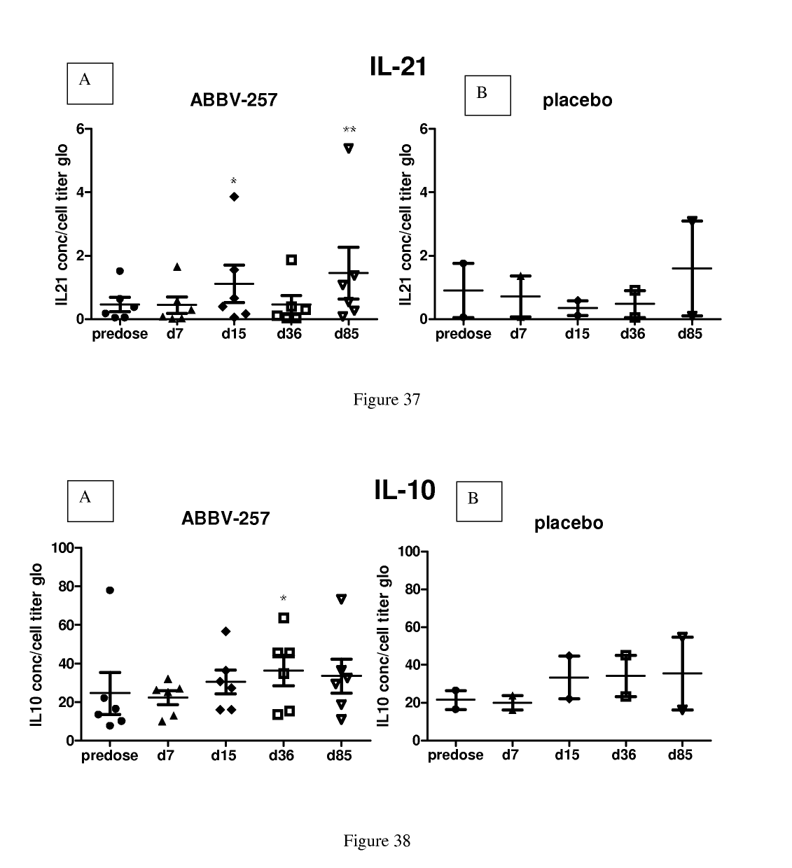

Most importantly, ex-vivo cytokine data for subjects subcutaneously administered ABBV-257 showed an increase in protein expression for cells stimulated with LPS or CD3/CD28. Data for ex vivo responses for IL-1Ra, GM-CSF, IL-21 and IL-10 for subcutaneous administration of ABBV-257 were consistent with data for ABT-122. See also FIGS. 35-38 for ABBV-257, and compare to Table 10 for ABT-122.

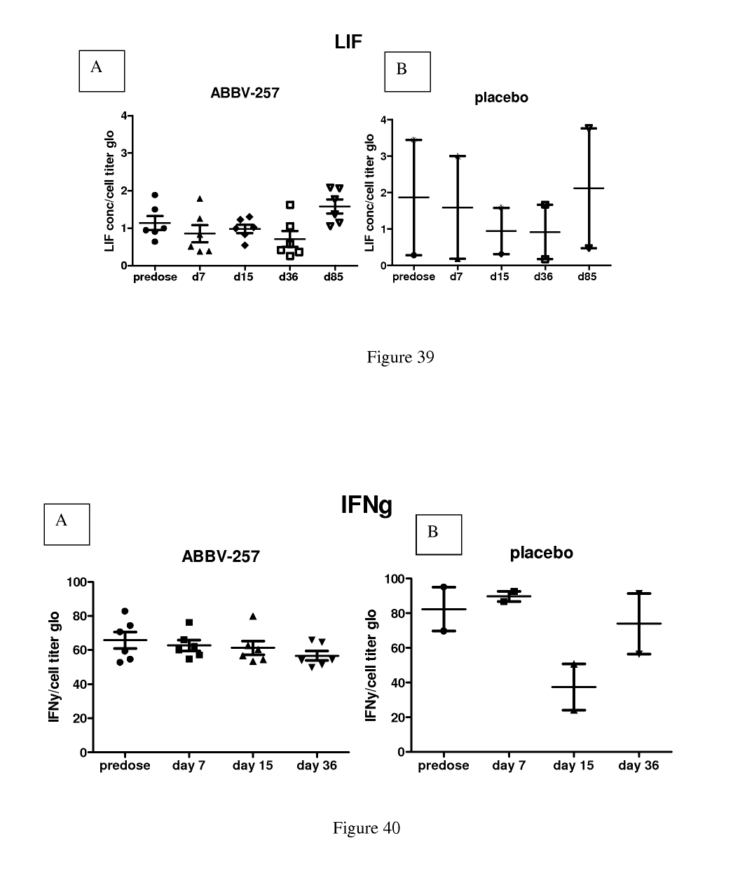

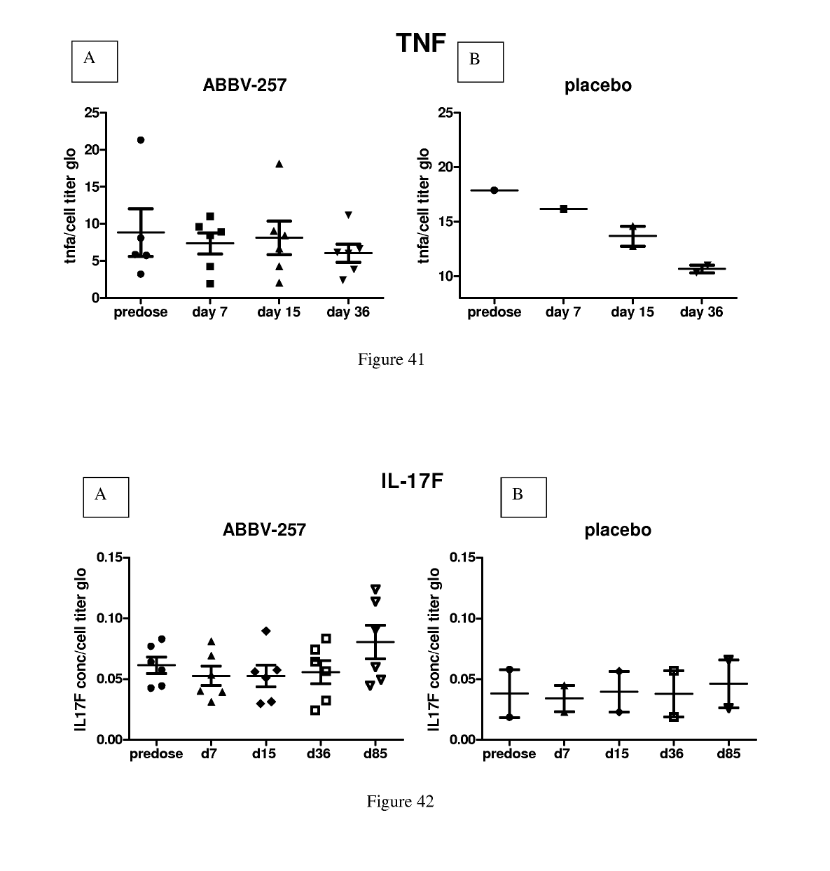

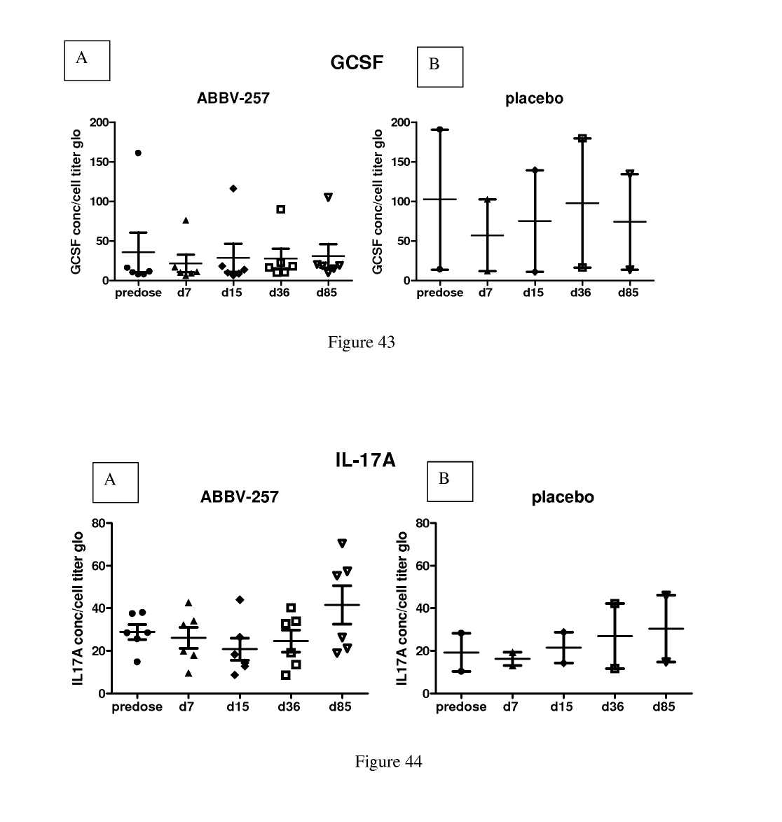

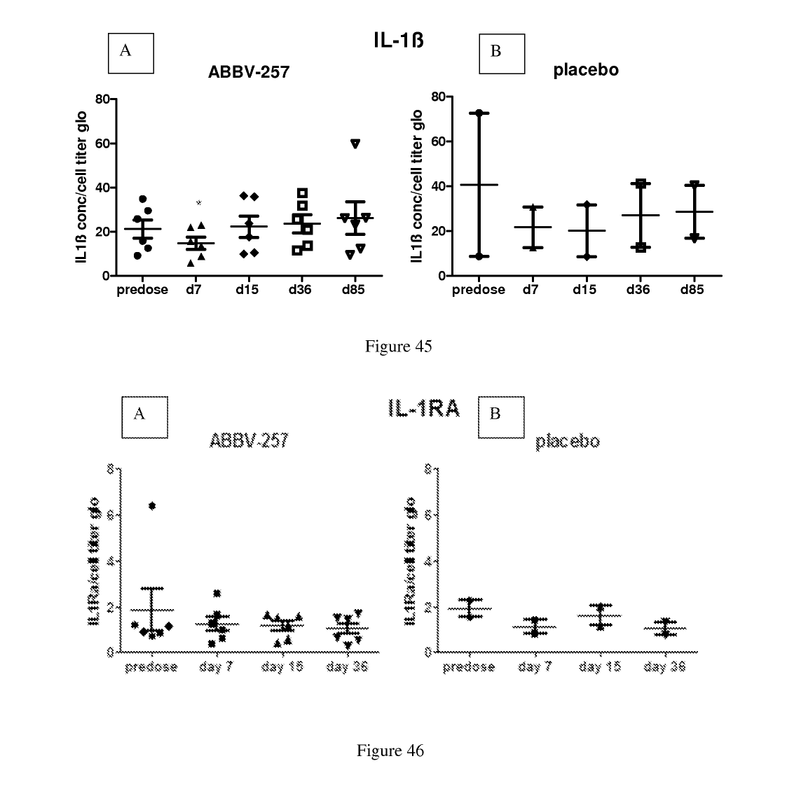

FIGS. 39-41 show ex vivo cytokine responses for LIF, IFNγ, and TNF in the ABBV-257 M14-355 study. Table 11 provides a summary of the ex vivo cytokine responses for ABV-257. These data may be compared to the IV data shown in Table 10 for ABT-122 to further elucidate the effects of ABBV-257 compared to ABT-122.

TABLE 11
Ex Vivo Cytokine Comparison For
Subjects Administered ABBV-257

| Cytokines | LPS stimulation | Anti-CD3/28 stimulation |
|---|---|---|
| IL-1Ra | ↑ | ND |
| GM-CSF | ↓ | ND |
| TNF | — | ND |
| IL-10 | ↑ | ND |
| IFNg | — | — |
| IL-21 | ND | ↑ |
| LIF | — | — |

ND = not done

Even considering the small group size and different TNF/IL-17 bispecific molecules, many of the same effects and significant changes that were observed following ABT-122 administration were observed after ABBV-257 administration. For example, data for ex vivo responses after ABT-122 and ABBV-257 administration show similar CXCR5 and CXCR4 expression changes, and similar changes in IL-10, IL-1Ra, IL-21 and GM-CSF ex vivo responses. These similar effects between ABT-122 and ABBV-257 administered subjects were not observed for G-CSF expression, along with changes in IFNγ, LIF and TNF ex vivo responses.

### INCORPORATION BY REFERENCE

The contents of all cited references (including literature references, patents, patent applications, databases and websites) that maybe cited throughout this application are hereby expressly incorporated by reference in their entirety for any purpose, as are the references cited therein. The practice of the present invention will employ, unless otherwise indicated, conventional techniques of immunology, molecular biology and cell biology, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

1. A method of selecting a first subject suffering from an inflammatory disorder for treatment with a combination therapy comprising an anti-TNF treatment and an anti-IL-17 treatment, the method comprising the steps of
a) contacting a sample from the first subject with one or more binding moieties that specifically bind at least one protein selected from the group consisting of: LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof;
b) detecting the interaction of the one or more binding moieties with the at least one protein, thereby detecting the relative abundance of the protein or a nucleic acid encoding the protein in the first subject sample;
c) comparing the relative abundance of the protein or nucleic acid in the first subject sample to the relative abundance of the protein or nucleic acid in a sample from a second subject, wherein the second subject does not suffer from the inflammatory disorder; and
d) selecting the first subject for the combination therapy comprising an anti-TNF treatment and an anti-IL-17 treatment if the relative abundance of the protein or nucleic acid in the first subject sample is modulated compared to the relative abundance of the protein or nucleic acid in the second subject sample.

2-14. (canceled)

15. A method of determining whether a candidate substance is an effective treatment for an inflammatory disorder in a first subject in need thereof comprising
a) contacting a sample from a second subject with the candidate substance, wherein the second subject suffers from the inflammatory disorder;
b) contacting the second subject sample with one or more binding moieties that specifically bind at least one protein selected from the group consisting of: LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof;
c) detecting the interaction of the one or more binding moieties with at least one protein, thereby detecting the relative abundance of the protein or nucleic acid in the second subject sample;
d) comparing the relative abundance of the protein or nucleic acid in the second subject sample to the relative abundance of the protein or nucleic acid in a third subject sample, wherein the third subject suffers from the inflammatory disorder and the third subject sample has not been contacted with the substance; and
e) determining that the substance is an effective treatment for an inflammatory disorder in the first subject if the relative abundance of the protein or nucleic acid in the second subject sample is modulated compared to the relative abundance of the protein or nucleic acid in the third subject sample.

16-25. (canceled)

26. The method of claim 1, wherein contacting is performed in vivo.

27. (canceled)

28. A method of determining whether a combination therapy comprising an anti-TNF treatment and an anti-IL-17 treatment is an effective treatment for an inflammatory disorder in a first subject in need thereof comprising
a) contacting a sample from a second subject with the combination therapy, wherein the second subject suffers from the inflammatory disorder;
b) contacting the second subject sample with one or more binding moieties that specifically bind at least one protein selected from the group consisting of: LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof;
c) detecting the interaction of the one or more binding moieties with the at least one protein, thereby detecting the relative abundance of the protein or nucleic acid in the second subject sample;
d) comparing the relative abundance of the protein or nucleic acid in the second subject sample to the relative abundance of the protein or nucleic acid in a third subject sample, wherein the third subject suffers from the inflammatory disorder and the third subject sample has not been contacted with the combination therapy; and
e) determining that the combination therapy is an effective treatment for an inflammatory disorder in the first subject if the relative abundance of the protein or nucleic acid in the second subject sample is modulated compared to the relative abundance of the protein or nucleic acid in the third subject sample.

29-40. (canceled)

41. The method of claim 1, wherein the combination therapy comprises an anti-TNF treatment that comprises an anti-TNF binding protein.

42. (canceled)

43. The method of claim 41, wherein the anti-TNF binding protein comprises an antibody, a fusion protein, a murine antibody, a human antibody, a humanized antibody, a bispecific antibody, a chimeric antibody, a Fab, a Fab′, a F(ab′)2, an ScFv, an SMIP, an affibody, an avimer, a versabody, a nanobody, a domain antibody, or an antigen binding fragment thereof.

44-49. (canceled)

50. The method of claim 1, wherein the combination therapy or candidate substance comprises anti-IL-17 treatment that comprises an anti-IL-17 binding protein.

51. (canceled)

52. The method of claim 50, wherein the anti-IL-17 binding protein comprises an antibody, a fusion protein, a murine antibody, a human antibody, a humanized antibody, a bispecific antibody, a chimeric antibody, a Fab, a Fab′, a F(ab′)2, an ScFv, an SMIP, an affibody, an avimer, a versabody, a nanobody, a domain antibody, or an antigen binding fragment thereof.

53-59. (canceled)

60. The method of claim 1, wherein the combination therapy comprises the administration of a multispecific binding protein that binds at least one of TNF and IL-17.

61. The method of claim 60, wherein the multispecific binding protein is selected from the group consisting of a dual variable domain immunoglobulin (DVD-Ig) molecule, a half-body DVD-Ig (hDVD-Ig) molecule, a triple variable domain immunoglobulin (TVD-Ig) molecule, a receptor variable domain immunoglobulin (rDVD-Ig) molecule, a polyvalent DVD-Ig (pDVD-Ig) molecule, a monobody DVD-Ig (mDVD-Ig) molecule, a cross over (coDVD-Ig) molecule, a blood brain barrier (bbbDVD-Ig) molecule, a cleavable linker DVD-Ig (clDVD-Ig) molecule, and a redirected cytotoxicity DVD-Ig (rcDVD-Ig) molecule.

62. The method of claim 60, wherein the multispecific binding protein binds TNFα and IL-17, and wherein the binding protein comprises at least one of:
a heavy chain amino acid sequence selected from SEQ ID NOs: 5, 11 and 24;
a light chain amino acid sequence selected from SEQ ID NOs: 8, 16, and 26;
a heavy chain constant region selected from SEQ ID NOs: 7, 15, and 25; or
a light chain constant region selected from SEQ ID NOs: 10, 20 and 30.

63. The method of claim 1, wherein the one or more binding moieties specifically bind nucleic acids.

64-94. (canceled)

95. A method of determining effectiveness of a combination therapy comprising an anti-TNF treatment and an anti-IL-17 treatment, and/or a selecting a subject suffering from an inflammatory disorder for treatment with the combination therapy, the method comprising
a) contacting a sample from the subject having with one or more binding moieties according to claim 28 that specifically bind a protein or a nucleic acid encoding the protein, wherein the protein is selected from the group consisting of: LIF, CXCL1, CXCL2, CXCL4, CXCL5, CXCL8, CXCL9, CXCL10, CCL2, CCL23, IL-1β, IL-1Ra, TNF, IL-6, IL-10, IL-17A, IL-17F, IL-21, IL-22, IFNγ, CXCR1, CXCR4, CXCR5, GM-CSF, GM-CSFR, G-CSF, G-CSFR protein or nucleic acid, or a homolog, portion or derivative thereof;
b) detecting the interaction of the one or more binding moieties with the protein or nucleic acid, thereby detecting the relative abundance of the protein or nucleic acid in the sample and/or expression of the protein on a cell surface of cells in the sample;
c) comparing the relative abundance or expression of the protein or nucleic acid to the relative abundance or expression of the protein or nucleic acid in a second subject sample, wherein the second subject does not suffer from the inflammatory disorder; and
d) selecting the subject for the combination therapy comprising an anti-TNF treatment and an anti-IL-17 treatment if the relative abundance or expression of the protein or nucleic acid in the subject sample is modulated compared to the relative abundance or expression of the protein or nucleic acid in the second subject sample.

96. (canceled)

97. A method of monitoring or calibrating a dosage in a subject being treated for an inflammatory disorder with a combination therapy comprising an anti-TNF treatment and an anti-IL-17 treatment, the method comprising the steps of
a) administering to the subject a first dose of a combination therapy comprising an anti-TNF treatment and an anti-IL-17 treatment;
b) determining a modulation of expression of one or more biomarkers in a sample from the subject, wherein the one or more biomarkers are gene products selected from the group consisting of LIF, CXCL1, CXCL2, CXCL5, CXCL9, CXCL10, CCL2, CCL23, IL-1Ra, TNF, IL-6, IL-10, IL-21, IL-22, IFNγ, CXCR4, CXCR5, GM-CSF, G-CSF and G-CSFR;
i) detecting the interaction of one or more binding moieties that specifically bind to the one or more biomarkers, thereby detecting the abundance of the one or more biomarkers in the subject sample; and
ii) obtaining a relative abundance of the one or more biomarkers in the subject sample by comparison to a baseline abundance of the biomarker;
and
c) administering a second dose of the combination therapy, wherein the second dose is determined depending on the relative abundance of the one or more biomarkers in the subject sample in response to the first dose.

98. The method of claim 97, wherein the second dose is equal to or greater than the first dose when the one or more biomarkers are gene products selected from the group consisting of LIF, IL-1RA, IL-10, IL-21 and CXCR5, and wherein the relative abundance of the one or more biomarkers in the subject sample in response to the first dose compared to the baseline abundance of the one or more biomarker is less.

99. The method of claim 97, wherein the second dose is equal to or greater than the first dose when the one or more biomarkers are gene products selected from the group consisting of CXCL1, CXCL2, CCL2, CXCL5, CXCL9, CXCL10, CCL23, TNF, IL-6, IL-22, IFNγ, CXCR4, GM-CSF, G-CSF and G-CSFR, and wherein the relative abundance of the one or more biomarkers in the subject sample in response to the first dose compared to the baseline abundance of the one or more biomarker is greater.

100. The method of claim 97, wherein the second dose is less than the first dose or treatment is discontinued when one or more biomarkers are gene products selected from the group consisting of LIF, IL-1RA, IL-10, IL-21 and CXCR5, and wherein the relative abundance of the one ore mores biomarker in the subject sample in response to the first dose compared to the baseline abundance of the one or more biomarker is greater.

101. The method of claim 97, wherein the second dose is less than the first dose or treatment is discontinued when one or more biomarkers are gene products selected from the group consisting of CXCL1, CXCL2, CCL2, CXCL5, CXCL9, CXCL10, CCL23, TNF, IL-6, IL-22, IFNγ, CXCR4, GM-CSF, G-CSF and G-CSFR and wherein the relative abundance of the one or more biomarkers in the subject sample in response to the first dose compared to the baseline abundance of the one or more biomarker is less.

102-103. (canceled)

104. A method of treating a subject suffering from an inflammatory disorder, the method comprising the steps of
a) determining a modulation of expression of one or more biomarkers in a sample from the subject, wherein the one or more biomarkers are gene products selected from the group consisting of LIF, CXCL1, CXCL2, CXCL5, CXCL9, CXCL10, CCL2, CCL23, IL-1Ra, TNF, IL-6, IL-10, IL-21, IL-22, IFNγ, CXCR4, CXCR5, GM-CSF, G-CSF and G-CSFR;
i) detecting the interaction of one or more binding moieties that specifically bind to the one or more biomarkers, thereby detecting the abundance of the biomarkers in the subject sample; and
ii) obtaining a relative abundance of the one or more biomarkers in the subject sample by comparison to a baseline abundance of the biomarker;
and
b) administering a dose of a combination therapy comprising an anti-TNF treatment and an anti-IL-17 treatment when the abundance of one or more biomarkers is modulated.

105. The method of claim 104, wherein the dose of combination therapy is administered to the subject when the one or more biomarkers are gene products selected from the group consisting of LIF, IL-1RA, IL-10, IL-21 and CXCR5 and wherein the abundance of the biomarker in the sample is less than the baseline abundance.

106. The method of claim 104, wherein the dose of combination therapy is administered to the subject when the one or more biomarkers are gene products selected from the group consisting of CXCL1, CXCL2, CCL2, CXCL5, CXCL9, CXCL10, CCL23, TNF, IL-6, IL-22, IFNγ, CXCR4, GM-CSF, G-CSF and G-CSFR and wherein the abundance of the biomarker in the sample is greater than the baseline abundance.

107. A method of determining an increased risk of an inflammatory disorder in a subject, the method comprising the steps of
a) determining a modulation of expression of one or more biomarkers in a sample from the subject, wherein the one or more biomarkers are gene products selected from the group consisting of LIF, CXCL1, CXCL2, CXCL5, CXCL9, CXCL10, CCL2, CCL23, IL-1Ra, TNF, IL-6, IL-10, IL-21, IL-22, IFNγ, CXCR4, CXCR5, GM-CSF, G-CSF and G-CSFR;
b) detecting the interaction of one or more binding moieties that specifically bind to the one or more biomarkers, thereby detecting the relative abundance of the one or more biomarkers in the subject sample; and
c) obtaining a relative abundance of the one or more biomarkers in the subject sample by comparison to a baseline abundance of the one or more biomarker;
wherein the subject has an increased risk of an inflammatory disorder when the abundance of the one or more biomarkers is modulated.

108. The method of claim 107, wherein the subject has an increased risk of an inflammatory disorder when the one or more biomarkers are gene products selected from the group consisting of LIF, IL-1RA, IL-10, IL-21 and CXCR5 and wherein the abundance of the biomarker in the sample is less than the baseline abundance.

109. The method of claim 107, wherein the subject has an increased risk of an inflammatory disorder when the one or more biomarkers are gene products selected from the group consisting of CXCL1, CXCL2, CCL2, CXCL5, CXCL9, CXCL10, CCL23, TNF, IL-6, IL-22, IFNγ, CXCR4, GM-CSF, G-CSF and G-CSFR and wherein the abundance of the biomarker in the sample is greater than the baseline abundance.

110-117. (canceled)


SEQUENCE LISTING (93 sequences)

SEQ ID NO: 1 - protein, 121 aa, Artificial Sequence
  Description of Artificial Sequence Synthetic polypeptide
EFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSS

SEQ ID NO: 2 - protein, 107 aa, Artificial Sequence
  Description of Artificial Sequence Synthetic polypeptide
QIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKR

SEQ ID NO: 3 - protein, 121 aa, Artificial Sequence
  Description of Artificial Sequence Synthetic polypeptide
QVQLQQSGAELVRPGTSVTLSCKASGYIFTDYEIHWVKQTPVHGLEWIGVNDPESGGTFYNQKFDGKAELTADKSSSTAYMELRSLTSEDSGVYYCTRYYRYESFYGMDYWGQGTSITVSS

SEQ ID NO: 4 - protein, 107 aa, Artificial Sequence
  Description of Artificial Sequence Synthetic polypeptide
QIVLTQSPAIMSASPGEKVTMTCSASSSISYIYWFQQKPGTSPKRWIYATFELASGVPARFSGSGSGTSYSLTISSMEAEDAATYYCHQRSSYPWTFGGGSKLEIKR

SEQ ID NO: 5 - protein, 252 aa, Artificial Sequence
  Description of Artificial Sequence Synthetic polypeptide
EFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSSGGGGSGGGGSQVQLQQSGAELVRPGTSVTLSCKASGYIFTDYEIHWVKQTPVHGLEWIGVNDPESGGTFYNQKFDGKAELTADKSSSTAYMELRSLTSEDSGVYYCTRYYRYESFYGMDYWGQGTSITVSS

SEQ ID NO: 6 - protein, 10 aa, Artificial Sequence
  Description of Artificial Sequence Synthetic peptide
GGGGSGGGGS

SEQ ID NO: 7 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 223 aa, Artificial Sequence
  Description of Artificial Sequence Synthetic polypeptide
QIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKRGGSGGGGSGQIVLTQSPAIMSASPGEKVTMTCSASSSISYIYWFQQKPGTSPKRWIYATFELASGVPARFSGSGSGTSYSLTISSMEAEDAATYYCHQRSSYPWTFGGGSKLEIKR

SEQ ID NO: 9 - protein, 9 aa, Artificial Sequence
  Description of Artificial Sequence Synthetic peptide
GGSGGGGSG

SEQ ID NO: 10 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 255 aa, Artificial Sequence
  Description of Artificial Sequence Synthetic polypeptide
EVQLVQSGAEVKKPGASVKVSCKASGYTFANYGIIWVRQAPGQGLEWMGWINTYTGKPTYAQKFQGRVTMTTDTSTSTAYMELSSLRSEDTAVYYCARKLFTTMDVTDNAMDYWGQGTTVTVSSGGGGSGGGGSEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 12 - protein, 124 aa, Artificial Sequence
  Description of Artificial Sequence Synthetic polypeptide
EVQLVQSGAEVKKPGASVKVSCKASGYTFANYGIIWVRQAPGQGLEWMGWINTYTGKPTYAQKFQGRVTMTTDTSTSTAYMELSSLRSEDTAVYYCARKLFTTMDVTDNAMDYWGQGTTVTVSS

SEQ ID NO: 13 - protein, 10 aa, Artificial Sequence
  Description of Artificial Sequence Synthetic peptide
GGGGSGGGGS

SEQ ID NO: 14 - protein, 121 aa, Artificial Sequence
  Description of Artificial Sequence Synthetic polypeptide
EVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 15 - protein, 330 aa, Artificial Sequence; identical to SEQ ID NO: 7
  Description of Artificial Sequence Synthetic polypeptide

SEQ ID NO: 16 - protein, 225 aa, Artificial Sequence
  Description of Artificial Sequence Synthetic polypeptide
DIQMTQSPSSLSASVGDRVTITCRASQDISQYLNWYQQKPGKAPKLLIYYTSRLQSGVPSRFSGSGSGTDFTLTISSLQPEDFATYFCQQGNTWPPTFGQGTKLEIKRGGSGGGGSGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 17 - protein, 108 aa, Artificial Sequence
  Description of Artificial Sequence Synthetic polypeptide
DIQMTQSPSSLSASVGDRVTITCRASQDISQYLNWYQQKPGKAPKLLIYYTSRLQSGVPSRFSGSGSGTDFTLTISSLQPEDFATYFCQQGNTWPPTFGQGTKLEIKR

SEQ ID NO: 18 - protein, 9 aa, Artificial Sequence
  Description of Artificial Sequence Synthetic peptide
GGSGGGGSG

SEQ ID NO: 19 - protein, 108 aa, Artificial Sequence
  Description of Artificial Sequence Synthetic polypeptide
DIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 20 - protein, 106 aa, Artificial Sequence; identical to SEQ ID NO: 10
  Description of Artificial Sequence Synthetic polypeptide

SEQ ID NO: 21 - protein, 257 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSEVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWNGYYSTHDFDSWGQGTTVTVSS

SEQ ID NO: 22 - protein, 121 aa, Artificial Sequence [sequence omitted]

SEQ ID NO: 23 - protein, 10 aa, Artificial Sequence
  Description of Artificial Sequence Synthetic peptide
GGGGSGGGGS

SEQ ID NO: 24 - protein, 126 aa, Artificial Sequence
  Description of Artificial Sequence Synthetic polypeptide
EVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWNGYYSTHDFDSWGQGTTVTVSS

SEQ ID NO: 25 - protein, 330 aa, Artificial Sequence [sequence omitted]

SEQ ID NO: 26 - protein, 225 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGSGGGGSGEIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKR

SEQ ID NO: 27 - protein, 108 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 28 - protein, 9 aa, Artificial Sequence
  Description of Artificial Sequence Synthetic peptide
GGSGGGGSG

SEQ ID NO: 29 - protein, 108 aa, Artificial Sequence
  Description of Artificial Sequence Synthetic polypeptide
EIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKR

SEQ ID NO: 30 - protein, 106 aa, Artificial Sequence; identical to SEQ ID NO: 10
  Description of Artificial Sequence Synthetic polypeptide

SEQ ID NO: 31 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 32 - protein, 133 aa, Homo sapiens [sequence omitted]

SEQ ID NO: 33 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 34 - protein, 4 aa, Artificial Sequence
  Description of Artificial Sequence Synthetic peptide
FGXG

SEQ ID NO: 35 - protein, 9 aa, Artificial Sequence
  Description of Artificial Sequence Synthetic peptide
CXXXXXXXX

SEQ ID NO: 36 - protein, 5 aa, Artificial Sequence
  Description of Artificial Sequence Synthetic peptide
LEWIG

SEQ ID NO: 37 - protein, 4 aa, Artificial Sequence
  Description of Artificial Sequence Synthetic peptide
WGXG

SEQ ID NO: 38 - protein, 6 aa, Artificial Sequence
  Description of Artificial Sequence Synthetic peptide
GGGGSG

SEQ ID NO: 39 - protein, 5 aa, Artificial Sequence
  Description of Artificial Sequence Synthetic peptide
GGSGG

SEQ ID NO: 40 - protein, 10 aa, Artificial Sequence
  Description of Artificial Sequence Synthetic peptide
GGGGSGGGGS

SEQ ID NO: 41 - protein, 9 aa, Artificial Sequence
  Description of Artificial Sequence Synthetic peptide
GGSGGGGSG

SEQ ID NO: 42 - protein, 10 aa, Artificial Sequence
  Description of Artificial Sequence Synthetic peptide
GGSGGGGSGS

SEQ ID NO: 43 - protein, 13 aa, Artificial Sequence
  Description of Artificial Sequence Synthetic peptide
GGSGGGGSGGGGS

SEQ ID NO: 44 - protein, 14 aa, Artificial Sequence
  Description of Artificial Sequence Synthetic peptide
GGGGSGGGGSGGGG

SEQ ID NO: 45 - protein, 15 aa, Artificial Sequence
  Description of Artificial Sequence Synthetic peptide
GGGGSGGGGSGGGGS

SEQ ID NO: 46 - protein, 6 aa, Artificial Sequence
  Description of Artificial Sequence Synthetic peptide
ASTKGP

SEQ ID NO: 47 - protein, 13 aa, Artificial Sequence
  Description of Artificial Sequence Synthetic peptide
ASTKGPSVFPLAP

SEQ ID NO: 48 - protein, 5 aa, Artificial Sequence
  Description of Artificial Sequence Synthetic peptide
TVAAP

SEQ ID NO: 49 - protein, 6 aa, Artificial Sequence
  Description of Artificial Sequence Synthetic peptide
RTVAAP

SEQ ID NO: 50 - protein, 12 aa, Artificial Sequence
  Description of Artificial Sequence Synthetic peptide
TVAAPSVFIFPP

SEQ ID NO: 51 - protein, 13 aa, Artificial Sequence
  Description of Artificial Sequence Synthetic peptide
RTVAAPSVFIFPP

SEQ ID NO: 52 - protein, 16 aa, Artificial Sequence
  Description of Artificial Sequence Synthetic peptide
AKTTPKLEEGEFSEAR

SEQ ID NO: 53 - protein, 17 aa, Artificial Sequence
  Description of Artificial Sequence Synthetic peptide
AKTTPKLEEGEFSEARV

SEQ ID NO: 54 - protein, 9 aa, Artificial Sequence
  Description of Artificial Sequence Synthetic peptide
AKTTPKLGG

SEQ ID NO: 55 - protein, 10 aa, Artificial Sequence
  Description of Artificial Sequence Synthetic peptide
SAKTTPKLGG

SEQ ID NO: 56 - protein, 6 aa, Artificial Sequence
  Description of Artificial Sequence Synthetic peptide
SAKTTP

SEQ ID NO: 57 - protein, 6 aa, Artificial Sequence
  Description of Artificial Sequence Synthetic peptide
RADAAP

SEQ ID NO: 58 - protein, 9 aa, Artificial Sequence
  Description of Artificial Sequence Synthetic peptide
RADAAPTVS

SEQ ID NO: 59 - protein, 12 aa, Artificial Sequence
  Description of Artificial Sequence Synthetic peptide
RADAAAAGGPGS

SEQ ID NO: 60 - protein, 27 aa, Artificial Sequence
  Description of Artificial Sequence Synthetic peptide
RADAAAAGGGGSGGGGSGGGGSGGGGS

SEQ ID NO: 61 - protein, 18 aa, Artificial Sequence
  Description of Artificial Sequence Synthetic peptide
SAKTTPKLEEGEFSEARV

SEQ ID NO: 62 - protein, 5 aa, Artificial Sequence
  Description of Artificial Sequence Synthetic peptide
ADAAP

SEQ ID NO: 63 - protein, 12 aa, Artificial Sequence
  Description of Artificial Sequence Synthetic peptide
ADAAPTVSIFPP

SEQ ID NO: 64 - protein, 6 aa, Artificial Sequence
  Description of Artificial Sequence Synthetic peptide
QPKAAP

SEQ ID NO: 65 - protein, 13 aa, Artificial Sequence
  Description of Artificial Sequence Synthetic peptide
QPKAAPSVTLFPP

SEQ ID NO: 66 - protein, 6 aa, Artificial Sequence
  Description of Artificial Sequence Synthetic peptide
AKTTPP

SEQ ID NO: 67 - protein, 13 aa, Artificial Sequence
  Description of Artificial Sequence Synthetic peptide
AKTTPPSVTPLAP

SEQ ID NO: 68 - protein, 6 aa, Artificial Sequence
  Description of Artificial Sequence Synthetic peptide
AKTTAP

SEQ ID NO: 69 - protein, 13 aa, Artificial Sequence
  Description of Artificial Sequence Synthetic peptide
AKTTAPSVYPLAP

SEQ ID NO: 70 - protein, 15 aa, Artificial Sequence
  Description of Artificial Sequence Synthetic peptide
GENKVEYAPALMALS

SEQ ID NO: 71 - protein, 15 aa, Artificial Sequence
  Description of Artificial Sequence Synthetic peptide
GPAKELTPLKEAKVS

SEQ ID NO: 72 - protein, 15 aa, Artificial Sequence
  Description of Artificial Sequence Synthetic peptide
GHEAAAVMQVQYPAS

SEQ ID NO: 73 - protein, 152 aa, Homo sapiens
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 74 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 75 - protein, 132 aa, Homo sapiens
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 76 - DNA, 1602 bp, Homo sapiens
agtcgtggccccaggtaatttcctcccaggcctccatggggttatgtataaaggcccccctagagctgggccccaaaacagcccggagcctgcagcccagccccacccagacccatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgcgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctccaaaaaaaaaaa

SEQ ID NO: 77 - DNA, 1184 bp, Homo sapiens
cacagagcccgggccgcaggcacctcctcgccagctcttccgctcctctcacagccgccagacccgcctgctgagccccatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatagaaaaaactcgtttgatttttggggggaaacaagggctacctttactggaaaatctggtgatttataaaaaaaaaaaaaaaa

SEQ ID NO: 78 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 79 - DNA, 1227 bp, Homo sapiens
ctttgcagataaatatggcacactagccccacgttttctgagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaagaaaaaaaaaaaa

SEQ ID NO: 80 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 81 - protein, 8 aa, Artificial Sequence
  Description of Artificial Sequence Synthetic peptide
GSGSGNGS

SEQ ID NO: 82 - protein, 8 aa, Artificial Sequence
  Description of Artificial Sequence Synthetic peptide
GSGSGSGS

SEQ ID NO: 83 - protein, 8 aa, Artificial Sequence
  Description of Artificial Sequence Synthetic peptide
GGSGSGSG

SEQ ID NO: 84 - protein, 6 aa, Artificial Sequence
  Description of Artificial Sequence Synthetic peptide
GGSGSG

SEQ ID NO: 85 - protein, 4 aa, Artificial Sequence
  Description of Artificial Sequence Synthetic peptide
GGSG

SEQ ID NO: 86 - protein, 8 aa, Artificial Sequence
  Description of Artificial Sequence Synthetic peptide
GGSGNGSG

SEQ ID NO: 87 - protein, 8 aa, Artificial Sequence
  Description of Artificial Sequence Synthetic peptide
GGNGSGSG

SEQ ID NO: 88 - protein, 6 aa, Artificial Sequence
  Description of Artificial Sequence Synthetic peptide
GGNGSG

SEQ ID NO: 89 - protein, 4 aa, Artificial Sequence
  Description of Artificial Sequence Synthetic peptide
GSGS

SEQ ID NO: 90 - protein, 3 aa, Artificial Sequence
  Description of Artificial Sequence Synthetic peptide
GSG

SEQ ID NO: 91 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 92 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 93 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP